SEQUENCE LISTING (11 sequences)

SEQ ID NO: 1 - DNA, 20 bp, Manihot esculenta
  source 1..20: mol_type = other DNA
ataaacgcttctcgcccatc

SEQ ID NO: 2 - DNA, 3804 bp, Arabidopsis thaliana
  source 1..3804: mol_type = other DNA
accgataaaatgaaaatcattaacggagtcacgtggggttcggttaaacgccgttaagtgtttttacgaagttcacgaggtttctacttttgtcttctcttattttaacgccaatcgcctttatttccatttgttataaagtcttctgacatttgtaatgatcgaattctgtatttacatatttgttatatcctatataaaatattatttgactttttcttaaattttgtggacttgtggacctattatgtttacatatttttgatgagaagatgccatatttcgaatatatatgtactagtgtaatctgaggaaattcaagttgaagccaatacatactttggaatttatatatttaaaaacaaaaaaaaactatattctataaataaatttacttatttactaattagtttgataaaattcagtttttaataatacttttatttgtatggttaaatatgctacttaacaaaatatttttaaatatatatattcatgagatttatccttcctttatatcgagcttaaaataaactattggtcacatgcatataatatgcatttatacataaagttctgatttatatgattaacattggtaacatgagtttagaagcaatatttatccgacacattttaaagaatgaacgaaaaatttaaaggtttcattcgtttaggtatacatacagaatttactcaagaactacaaaactttgatattaaaatatcaaatgatgccgatcatttgtcttaagatgtaaattaaaagagagaaaaaaaaacacaaaaatgaaaatgtgcagttgttttaataacggcgacctaactttctttatgaggacccaagacctacaccacctccaaaattggaccacctttcgatttcactactcattgagagtctcatacatccacacgagtagctaaactatttttttttaagcataaacaaagctggaattcattttttttttctttctaatgtaatcaaagcttaaattcaacattccttataaaatatgttataaatgataatgtttaggcaaaaacataacatcatgtcacatgatttttttttaatagaaaaccagtacggcgttcgacaatatcgatacacagagtcgttttcctaacgaacctaacgcatgacacagaactagttgaatttgtaagataaaaactggtttgacatgtatcggtatcgaactcaaatacgagaaattacctctggttcgggttttttcattatcaaactaatatgctacaaagtgagaaagtcaggctcagaaggaggtatgaagcacaaagcccacttggaatctccgaagattattgggccttaaataaggccagaaaatataattgaatgataaaaggaaacttttcttacatttttggcaaaatacgttccttttattattggtgtattttaatttagtttaattacttcacaacaattggctgtttttctccagaagaacactaaagacaaaaattaaaccctcacactcacagagaccggagaagaagcagagagaaagagagagagagagattcaccggtgataacattgaagcagaaagattctccggtgataacattgaagctgaggagcaatgtatccgactggtcaacaggttggttgaaaaaaactcagttcaattgcaaaatggaactagattagttgggaaaaaacgaacgaactttagtctatgatctctcttaaacgatgaagtctctctgaatttttctgtgaatctggagaaaagactcgtgctttattgtttattgatgtatctactctcgacattttgtttctttttttattgcatgcgaccacattggattaaatttgagggtttgtgtttcagttttttacgaattttaagagttagggtttatacaggaatagtttttgagtcatttttgtatccaattttgtagatttcgtttcagaccactccgttgaatgttcaagatccaacaaggatgatgaatttggatcaatcttcaccagttgcaagaaatgaaactcagaatggaggaggcattgctcacgctgagtttgctatgttcaattccaagaggcttgaatctgatcttgaagctatgggtaacaaaatcaaacagcatgaggataatctcaagtttctcaagtctcagaaaaacaaaatggatgaagctatcgtcgacttgcaaggttctgagatatcatagacttttacgtttcagtgatttatgttgattgagattatccaatatgtctttgtttcgtttcttctgcagttcatatgagtaagcttaactcgtctcctactcctagaagtgaaaactctgataacagtcttcagggtgaagatatcaatgcacagatccttcgccacgaaaactcagctgctggagttttaagtctggttgagactcttcatggtgctcaagcttctcagttgatgttgacaaaaggtgttgttggtgttgtagcgaaacttgggaaggtcaatgatgaaaatctcagccagttagttccatttctctcactttttcagattaccttcaacttctcaatgccactgattatataagaaacctttcttgcaatggtttttgtgatctgaaactaactagttctctctcgtggtactttaaaaatgcaggattttgtcgaattatttggggactcgctcaatgttggcagttgtatgcaggaattatgaaagtgttacggctttagaggcttatgataaccatggcaacattgatataaatgctggccttcattgccttggctcttcgattggaagagagattggagacagttttgatgccatctgccttgaaaacctgaggtactaatttttgtttgaatcttttacatgtattacaaatttggttaggtacacttcatctgcagtccttttctaattgcttcatttgatgacttttaggccgtatgttggtcagcacatagctgatgatctgcaaagaaggcttgatcttctgaaaccaaaattacctaatggtgagtgtcctcccgggtttctcggatttgcagtgaatatgatacagatcgatccagcttacttgctctgtgtcacatcatatggatatggtcttcgtgagaccttgttctacaatctattctctcgccttcaagtttacaaaacaagggccgatatgattagtgccctcccatgcataagtgatggtgcggtatctttggatggaggaatcatcaggaaaacggggatcttcaatcttggaaaccggtaagattctcacaattttataacctctgaacttatttccctctttggattcttccatttccttcttgctcttatccgctgtcctgaactgaaatcaatatgattttcagtgatgaggtgaacgtgagatttgcaaagccaactgcttctcgcacgatggacaactatagtgaggcggagaagaaaatgaaagagctgaaatggaaaaaggagaaaacgctggaggacattaagcgggagcaagtgctccgtgagcatgccgtcttcaactttggcaagaagaaagaagaatttgttcgatgcttggctcagagttcatgcactaatcaggtattaaaactagttttcatgtatcttctgacccttctcttctatcatagtatatgtctgttgaagcatatcatgaaattgtctatatgatcatatctttgtttcagttttaattcaaagcttcaaatacctctatctgaaagttttatatggctaacattggtctgtttatgtgttggtggtgccccagccaatgaacacacccaga

SEQ ID NO: 3 - DNA, 20 bp, synthetic construct
  source 1..20: mol_type = other DNA
ctagctatgttgtgcaatga

SEQ ID NO: 4 - DNA, 20 bp, synthetic construct
  source 1..20: mol_type = other DNA
taggggaggaatccagggaa

SEQ ID NO: 5 - DNA, 504 bp, synthetic construct
  source 1..504: mol_type = other DNA
gagaaaccgtacaagtgtcctgagtgcggtaaaagtttctctaggagtgatgacctggttagacaccaaaggacccacacaggcgagaagccctataaatgtcctgaatgtggtaagagctttagtcaatccagcaatctagtgcgtcatcagaggacgcataccggcgaaaagccctataaatgtcctgagagtggtaaaagtttctcacaaagcggacatttaaccgagcatcagaggactcatacaggggaaaaaccatacaaatgtcccgagtgcggcaagtcatttagtaggagtgacaagctcgtgcgacaccaacgaacccatacaggagagaagccatacaaatgtccggagtgtggaaaaagttttagtacatcaggaaatttggtaagacatcagcgtacccacaccggtgagaaaccttacaaatgccctgaatgtggaaagtctttcagtaggagggatgagctgaatgtccaccagagaacgcatacgggg

SEQ ID NO: 6 - DNA, 1170 bp, synthetic construct
  source 1..1170: mol_type = other DNA
gctagggacagcaaagtggagaacaaaacaaagaagctgagagtgttcgaagccttcgccggcattggcgcccagagaaaggccctggagaaagtgaggaaggacgagtacgagatcgtgggactggccgagtggtatgtgcccgccatcgtcatgtaccaggccatccataacaacttccacaccaagctcgagtacaagtccgtcagcagagaggagatgatcgactacctcgagaacaagaccctgtcctggaacagcaagaaccccgtcagcaacggatactggaagaggaagaaggatgacgagctgaagatcatctacaacgccatcaagctgtccgaaaaggagggcaacattttcgacatcagggacctctacaagaggacactgaagaacatcgacctgctcacctacagcttcccttgccaggacctgagccagctaggcatccagaagggcatgaagaggggaagcggcaccagatccggcctgctctgggagatcgaaagagccctggactccaccgagaagaacgacctgcctaagtacctcctcatggagaacgtgggagccctgctgcacaagaagaacgaggaggagctgaaccaatggaagcagaagctggagagcctgggctaccagaacagcatcgaagtcctcaatgctgccgatttcggatccagccaggccaggaggagagtgttcatgatctccaccctcaatgagttcgtggaactgcctaagggcgacaagaagcccaagagcatcaaaaaggtgctgaacaagatcgtgagcgagaaggacatcctcaacaacctgctgaaatacaacctcaccgaattcaagaagaccaagtccaacatcaacaaggccagcctgatcggctactccaagttcaactccgagggctatgtgtacgaccccgagttcacaggccccacactgacagctagcggcgccaactccaggatcaagatcaaggacggcagcaacatcaggaagatgaacagcgacgagaccttcctgtacatcggctttgacagccaggacggcaagagagtgaacgaaatcgagttcctgaccgagaaccagaagatcttcgtgtgtggcaacagcatcagcgtggaggtgctggaggccatcattgacaagatcggcggccctagc

SEQ ID NO: 7 - DNA, 1062 bp, Nicotiana tabacum
  source 1..1062: mol_type = other DNA
gagacaattcgtttgcccaaacctatgattgggtttggggttcctaccgaaccacttccagcaatggttcgaagaactcttcccgagcaagctgttggacccccgtttttctactatgaaaatgtggctctagctccaaagggtgtgtgggacacaatttctagatttttgtacgatattgagccagagtttgtcgactccaaatatttttgtgctgctgcaagaaaaaggggttatattcataatctgccggttgaaaatagatttcctttgtttccacttgccccacgtaccattcatgaggcacttcccctatcgaagaaatggtggccatcttgggacccacggacaaagttaaattgtttgcaaacagccattggaagtgcacaattgacgaataggattaggaaagctgtggaggactttgatggtgagccaccaatgagagttcagaagtttgttcttgatcaatgtcggaagtggaatttggtgtgggttgggagaaacaaagttgctcctttggagcctgatgaagttgaaatgctactgggatttccaaagaaccatactaggggaggtggtataagtaggaccgatagatacaagtcgcttggtaattcattccaggttgacactgtggcataccatttgtcggtattgaaagacttgtttcccggtggtatcaatgtcttatcactcttctctggaattggcggtggtgaagttgctctttatcgtcttggtattccactaaacacagtggtttctgtggaaaaatctgaagtcaacagggatattgtgagaagctggtgggagcaaactaatcagagagggaaccttatacattttaacgatgtgcagcagctgaatggagaccgcttggagcaactgatagagtcatttggaggatttgatttggtaattggtggaagcccgtgcaataaccttgcaggcagtaacagagtgagcagggatgggcttgaaggcaaagagtcttccctattttatgactatgttcggatattggacttggtcaagtccataatgtccagacataaacat

SEQ ID NO: 8 - DNA, 20 bp, synthetic construct
  source 1..20: mol_type = other DNA
cgctctcaactgtacttcat

SEQ ID NO: 9 - DNA, 20 bp, synthetic construct
  source 1..20: mol_type = other DNA
ccgatgaataagagcgctag

SEQ ID NO: 10 - DNA, 20 bp, synthetic construct
  source 1..20: mol_type = other DNA
agacgatgaaagagccgaag

SEQ ID NO: 11 - DNA, 20 bp, synthetic construct
  source 1..20: mol_type = other DNA
cactcgattgcagatttttg

(19) United States
(12) Patent Application Publication
(10) Pub. No.: US 2025/0230463 A1
(43) Pub. Date: Jul. 17, 2025

(54) ENGINEERING DISEASE RESISTANCE BY EDITING THE EPIGENOME

(71) Applicant: Donald Danforth Plant Science Center, St. Louis, MO (US)

(72) Inventors: Rebecca Bart, St. Louis, MO (US); Kira Veley, St. Louis, MO (US); James Carrington, St. Louis, MO (US); Zhu-Jyh Daniel Lin, St. Louis, MO (US)

(21) Appl. No.: 18/686,140

(22) PCT Filed: Aug. 26, 2022
(86) PCT No.: PCT/US2022/075536

§ 371 Date: Feb. 23, 2024

Related U.S. Application Data
(60) Provisional application No. 63/237,218, filed on Aug. 26, 2021.

(51) Int. Cl. C12N 15/82; C12N 9/10; C12N 9/22; C12N 15/11

(52) U.S. Cl. CPC C12N 15/8281; C12N 9/1007; C12N 9/22; C12N 15/11; C12N 15/8201; C12N 15/8283; C07K 2319/81; C12N 2310/20; C12Y 201/01037

(57) ABSTRACT

The present disclosure provides engineered DNA methylation systems and methods for epigenetically modulating the expression of one or more plant pathogen susceptibility genes. The engineered DNA methylation systems can be used to generate epigenetically modified disease-resistant plants.

### CROSS-REFERENCE TO RELATED APPLICATIONS

This application claims priority from Provisional Application No. 63/237,218, filed Aug. 26, 2021, the entire contents of which are hereby incorporated by reference.

### SEQUENCE LISTING

This application contains a Sequence Listing that has been submitted in .XML format via PatentCenter and is hereby incorporated by reference in its entirety. The XML is named 077875_737753_Sequence_Listing and is 20 kilobytes in size.

### FIELD OF THE INVENTION

The present disclosure provides systems and methods of generating epigenetically modified disease-resistant plants.

### BACKGROUND OF THE INVENTION

Plant diseases can drastically abate the crop yields and the degree of disease outbreak is getting severe around the world. Therefore, plant disease management has always been and continues to be one of the main objectives of any crop improvement program. Crop improvement efforts to control plant diseases include breeding and biotechnology. The former relies on screening for resistant lines under field conditions where disease pressure is often unpredictable. In addition, previous reports suggest that different plant varieties display variable levels of tolerance depending upon the environment in which they are grown. This further complicates breeding efforts. Nevertheless, the predicted economic gains from disease-resistant plants are incalculable.

Accordingly, there is a need for disease-resistant plants, and methods of generating disease-resistant plants.

### SUMMARY OF THE INVENTION

One aspect of the instant disclosure encompasses an expression construct for methylating a target nucleic acid sequence in a plant pathogen susceptibility gene. The expression construct comprises a promoter operably linked to a nucleic acid sequence encoding an engineered protein. The engineered protein comprises a methylation polypeptide comprising a DNA methylation domain of a DNA methylation protein linked to a targeting polypeptide comprising a sequence-specific DNA binding domain, wherein the DNA binding domain binds a target DNA sequence in a target methylation locus in a polynucleotide encoding a plant pathogen susceptibility gene. Binding of the DNA binding domain to the target DNA sequence can target the engineered protein to the target locus, thereby mediating methylation of one or more methylation sites in the target locus, thereby modulating the expression of the plant pathogen susceptibility gene.

In some aspects, the targeting polypeptide is fused to the methylation polypeptide. In other aspects, the targeting polypeptide comprises an epitope and the methylation polypeptide comprises an affinity polypeptide that specifically binds to the epitope, and wherein binding of the affinity polypeptide to the epitope links the targeting polypeptide to the methylation polypeptide. The epitope can be multimerized.

In some aspects, the targeting polypeptide is a programmable targeting protein comprising a programmable, sequence-specific DNA-binding domain. The programmable targeting polypeptide can be an RNA-guided clustered regularly interspersed short palindromic repeats (CRISPR)/CRISPR-associated (Cas) (CRISPR/Cas) nuclease system, a zinc finger nuclease (ZFN), a transcription activator-like effector nuclease (TALEN), a meganuclease, a ssDNA-guided Argonaute endonuclease, a meganuclease, a rare-cutting endonuclease, or any combination thereof. In one aspect, the programmable targeting protein is a CRISPR/Cas nuclease system comprising a nuclease-deficient CAS9 protein (dCAS9) and a guide RNA (gRNA). In another aspect, the programmable targeting protein is a zinc finger DNA binding domain. In yet another aspects, the targeting polynucleotide comprises a TALE protein.

The engineered protein can comprise more than one methylation polypeptide linked to a targeting polypeptide programmed to target the more than one methylation polypeptide to the target methylation loci. Alternatively, the engineered protein can comprise a methylation polypeptide and more than one targeting polypeptide engineered to bind one or more target DNA sequence.

The engineered protein can mediate methylation of more than one target methylation locus. The engineered protein can also modulate the expression of more than one plant pathogen susceptibility gene.

The methylation polypeptide can methylate CpG, CpHpG, or CpHpH methylation sites, or any combination thereof. In some aspects, the methylation polypeptide methylates CpG, CpHpG, or CpHpH methylation sites, or any combination thereof to thereby remove histone proteins. The engineered protein can comprise a DNA methylation domain of a methylation protein selected from SUVH2, SUVH9, DMS3, DRM2, DRM3, NRPE1, NRPD1, CLSY1, NRPD2, RDR2, DCL3, AGO4, DRD1, RDM1, DMS4, KTF1, IDN2, SUVR2, MQ1, and any combination thereof.

In some aspects, the engineered protein comprises a DNA methylation domain of a DMS3 protein. In one aspect, the DMS3 protein is encoded by a nucleic acid sequence comprising at least about 75% or more, at least about 85% or more, at least about 95% or more, or 100% sequence identity with a nucleic acid sequence of SEQ ID NO: 2.

In some aspects, the engineered protein comprises a DNA methylation domain of a DRM2 protein. In one aspect, the DRM2 protein is encoded by a nucleic acid sequence comprising at least about 75% or more, at least about 85% or more, at least about 95% or more, or 100% sequence identity with a nucleic acid sequence of SEQ ID NO: 7.

In some aspects, the engineered protein comprises a DNA methylation domain of a MQ1 protein. In one aspects, the MQ1 protein is encoded by a nucleic acid sequence comprising at least about 75% or more, at least about 85% or more, at least about 95% or more, or 100% sequence identity with a nucleic acid sequence of SEQ ID NO: 6.

The pathogen can be a viral, bacterial, oomycete, animal, fungal pathogen, or any combination thereof. In some aspects, the pathogen is a viral pathogen. In some aspects, the pathogen is a bacterial pathogen.

In some aspects, the plant is cassava. When the plant is cassava, the susceptibility gene can be MeSWEET10a. In some aspects, the plant is cassava, the susceptibility gene is MeSWEET10a, and the pathogen is a bacterial pathogen that causes cassava bacterial blight (CBB). The pathogen that causes CBB is can be a Xanthomonas sp. In some aspects, the plant is cassava, the plant pathogen susceptibility gene is MeSWEET10a, and the pathogen is a Xanthomonas sp.

When the plant is cassava, the plant pathogen susceptibility gene can also be nCBP-1, nCBP-2, or combinations thereof. In some aspects, the plant pathogen susceptibility gene is nCBP-1 and nCBP-2. In other aspects, the plant is cassava, the plant pathogen susceptibility gene is nCBP-1 and nCBP-2, and the pathogen is a viral pathogen that causes cassava brown streak disease. The viral pathogen that causes cassava brown streak disease can be selected from cassava brown streak virus (CBSV), Uganda CBSV, or a combination thereof. In some aspects, the plant is cassava, the susceptibility gene is nCBP-1 and nCBP-2, and the pathogen is CBSV.

The methylation polypeptide of the engineered protein can comprise a DNA methylation domain of a DMS3 protein fused to a zinc finger DNA binding domain programmed to target the engineered protein to a locus in a promoter region of a cassava MeSWEET10a gene. In some aspects, the DMS3 protein (or methylation polypeptide) is encoded by a nucleic acid sequence comprising at least about 75% or more, at least about 85% or more, at least about 95% or more, or 100% sequence identity with a nucleic acid sequence of SEQ ID NO: 2 and wherein the programmable targeting protein (or targeting polypeptide) comprises an amino acid sequence comprising at least about 75% or more, at least about 85% or more, at least about 95% or more, or 100% sequence identity with the amino acid sequence of SEQ ID NO: 5.

The methylation polypeptide of the engineered protein can comprise a DNA methylation domain of an MQ1 protein fused to a nuclease-deficient CAS9 protein (dCAS9) of a CRISPR/Cas nuclease system comprising a gRNA comprising a sequence which binds to a nucleotide sequence in the target nucleic acid sequence to thereby target the engineered protein to a locus in a promoter region of a cassava MeSWEET10a gene. The MQ1 protein can be encoded by a nucleic acid sequence comprising at least about 75% or more, at least about 85% or more, at least about 95% or more, or 100% sequence identity with a nucleic acid sequence of SEQ ID NO: 6 and wherein the gRNA is selected from a gRNA selected from a gRNA comprising SEQ ID NO: 3, a gRNA comprising SEQ ID NO: 4, or a combination thereof.

The methylation polypeptide of the engineered protein can comprise a DNA methylation domain of a DRM2 methylation polypeptide comprising an affinity polypeptide and a dCAS9 protein of a CRISPR/Cas nuclease system comprising a gRNA comprising a sequence which binds to a nucleotide sequence in the target nucleic acid sequence to thereby target the engineered protein to a locus in a promoter region of a cassava MeSWEET10a gene, wherein the dCas9 protein comprises an epitope that specifically binds to the affinity polypeptide. The gRNA can be selected from a gRNA selected from a gRNA comprising the nucleic acid sequence of SEQ ID NO: 3, a gRNA comprising the nucleic acid sequence of SEQ ID NO: 4, or a combination thereof.

The methylation polypeptide of the engineered protein can comprise a DNA methylation domain of a DRM2 methylation polypeptide comprising an affinity polypeptide and a dCAS9 protein of a CRISPR/Cas nuclease system comprising a gRNA comprising a sequence which binds to a nucleotide sequence in the target nucleic acid sequence to thereby target the engineered protein to a locus in a promoter region of a cassava nCBP1 gene, wherein the dCas9 protein comprises a multimerized epitope that specifically binds to the affinity polypeptide. The gRNA can be selected from a gRNA selected from a gRNA comprising the nucleic acid sequence of SEQ ID NO: 8, a gRNA comprising the nucleic acid sequence of SEQ ID NO: 9, or a combination thereof.

The engineered protein can comprise a DRM2 methylation polypeptide comprising an affinity polypeptide and a dCAS9 protein of a CRISPR/Cas nuclease system comprising a gRNA comprising a sequence which binds to a nucleotide sequence in the target nucleic acid sequence to thereby target the engineered protein to a locus in a promoter region of a cassava nCBP2 gene, wherein the dCas9 protein comprises a multimerized epitope that specifically binds to the affinity polypeptide. The gRNA can be selected from a gRNA selected from a gRNA comprising the nucleic acid sequence of SEQ ID NO: 10, a gRNA comprising the nucleic acid sequence of SEQ ID NO: 11, or a combination thereof.

Another aspect of the instant disclosure encompasses an expression construct for methylating a target nucleic acid sequence in a plant pathogen susceptibility gene. The expression construct comprises a promoter operably linked to a nucleic acid sequence encoding an engineered protein. The engineered protein comprises a programmable targeting polypeptide comprising a programmable sequence-specific DNA binding domain, wherein the programmable DNA binding domain binds a target DNA sequence in a target methylation locus in a polynucleotide encoding a plant pathogen susceptibility gene. The programmable targeting protein comprises a nuclease-deficient CAS9 protein (dCAS9) and optionally an epitope; and one or more guide RNA. The engineered protein also comprises a methylation polypeptide comprising a DNA methylation domain of a DRM2 protein, a DMS3 protein, or an MQ1 protein. The methylation polypeptide is fused to the targeting polypeptide or fused to an affinity polypeptide that specifically binds to the epitope.

Yet another aspect of the instant disclosure encompasses an expression construct for methylating a target nucleic acid sequence in a plant pathogen susceptibility gene. The expression construct comprises a promoter operably linked to a nucleic acid sequence encoding an engineered protein. The engineered protein comprises a programmable targeting polypeptide comprising a programmable sequence-specific DNA binding domain of a zinc finger DNA binding protein programmed to specifically bind one or more target DNA sequence in a target methylation locus in a polynucleotide encoding a plant pathogen susceptibility gene, wherein the targeting polypeptide optionally comprises an epitope. The engineered protein also comprises a methylation polypeptide comprising a DNA methylation domain of a DMS3 protein, a DRM2 protein, an MQ1 protein. The methylation polypeptide is fused to the targeting polypeptide or fused to an affinity polypeptide that specifically binds to the epitope.

An additional aspect of the instant disclosure encompasses an expression construct for methylating a target nucleic acid sequence in a plant pathogen susceptibility gene. The expression construct comprises a promoter operably linked to a nucleic acid sequence encoding an engineered protein, and the engineered protein comprises a programmable targeting polypeptide comprising a programmable sequence-specific DNA binding domain of a TALE DNA binding protein programmed to specifically bind one or more target DNA sequence in a target methylation locus in a polynucleotide encoding a plant pathogen susceptibility gene, wherein the targeting polypeptide optionally comprises an epitope. The engineered protein also comprises a methylation polypeptide comprising a DNA methylation domain of a DMS3 protein, a DRM2 protein, an MQ1 protein. The methylation polypeptide is fused to the targeting polypeptide or fused to an affinity polypeptide that specifically binds to the epitope.

One aspect of the instant disclosure encompasses one or more vectors comprising one or more expression constructs for methylating a target nucleic acid sequence in a plant pathogen susceptibility gene. The constructs comprise a promoter operably linked to a nucleic acid sequence encoding an engineered protein. The constructs and the engineered protein can be as described herein above.

Yet another aspect of the instant disclosure encompasses a plant or plant cell comprising one or more expression constructs for methylating a target nucleic acid sequence in a plant pathogen susceptibility gene or one or more vectors comprising the one or more constructs. The constructs comprise a promoter operably linked to a nucleic acid sequence encoding an engineered protein. The constructs, the vectors, and the engineered protein can be as described herein above.

Another aspect of the instant disclosure encompasses a plant or plant cell comprising one or more methylated sites in a methylation locus in a plant pathogen susceptibility gene.

In some aspects, the plant is cassava. When the plant is cassava, the susceptibility gene can be MeSWEET10a. In some aspects, the plant is cassava, the susceptibility gene is MeSWEET10a, and the pathogen is a bacterial pathogen that causes cassava bacterial blight (CBB). The pathogen that causes CBB is can be a Xanthomonas sp. In some aspects, the plant is cassava, the plant pathogen susceptibility gene is MeSWEET10a, and the pathogen is a Xanthomonas sp.

When the plant is cassava, the plant pathogen susceptibility gene can also be nCBP-1, nCBP-2, or combinations thereof. In some aspects, the plant pathogen susceptibility gene is nCBP-1 and nCBP-2. In other aspects, the plant is cassava, the plant pathogen susceptibility gene is nCBP-1 and nCBP-2, and the pathogen is a viral pathogen that causes cassava brown streak disease. The viral pathogen that causes cassava brown streak disease can be selected from cassava brown streak virus (CBSV), Uganda CBSV, or a combination thereof. In some aspects, the plant is cassava, the susceptibility gene is nCBP-1 and nCBP-2, and the pathogen is CBSV.

One aspect of the instant disclosure encompasses a disease-resistant cassava plant. The cassava plant comprises one or more methylated sites in a promoter region of a MeSWEET10a susceptibility gene. The cassava plant is resistant to a Xanthomonas sp. that causes cassava bacterial blight (CBB).

Another aspect of the instant disclosure encompasses disease-resistant cassava plant. The cassava plant comprises one or more methylated sites in a promoter region of an nCBP-1 gene susceptibility and one or more methylated sites in a promoter region of an nCBP-2 susceptibility gene. The cassava plant is resistant to a viral pathogen that causes cassava brown streak disease. In some aspects, the viral pathogen that causes cassava brown streak disease is selected from cassava brown streak virus (CBSV), Uganda CBSV, or a combination thereof.

One aspect of the instant disclosure encompasses a disease-resistant cassava plant. The cassava plant comprises one or more methylated sites in a promoter region of an nCBP-1 gene susceptibility and one or more methylated sites in a promoter region of an nCBP-2 susceptibility gene. The cassava plant is resistant to CBSV.

Yet another aspect of the instant disclosure encompasses a method of generating a disease resistant or tolerant plant. The method comprises the steps of (a) introducing one or more expression constructs expressing an engineered protein or one or more vectors comprising the one or more expression constructs into a plant or plant cell; (b) cultivating the plant or plant cell under conditions sufficient for the engineered protein is targeted to the target methylation loci in the one or more plant pathogen susceptibility genes, thereby generating an engineered plant or plant cell comprising one or more methylated loci, thereby generating the disease resistant or tolerant plant; and (c) optionally removing the one or more expression or one or more one or more vectors from the plant or plant cell. The constructs, the vectors, and the engineered protein can be as described herein above.

In some aspects, the plant is cassava. When the plant is cassava, the susceptibility gene can be MeSWEET10a. In some aspects, the plant is cassava, the susceptibility gene is MeSWEET10a, and the pathogen is a bacterial pathogen that causes cassava bacterial blight (CBB). The pathogen that causes CBB is can be a Xanthomonas sp. In some aspects, the plant is cassava, the plant pathogen susceptibility gene is MeSWEET10a, and the pathogen is a Xanthomonas sp.

When the plant is cassava, the plant pathogen susceptibility gene can also be nCBP-1, nCBP-2, or combinations thereof. In some aspects, the plant pathogen susceptibility gene is nCBP-1 and nCBP-2. In other aspects, the plant is cassava, the plant pathogen susceptibility gene is nCBP-1 and nCBP-2, and the pathogen is a viral pathogen that causes cassava brown streak disease. The viral pathogen that causes cassava brown streak disease can be selected from cassava brown streak virus (CBSV), Uganda CBSV, or a combination thereof. In some aspects, the plant is cassava, the susceptibility gene is nCBP-1 and nCBP-2, and the pathogen is CBSV.

An additional aspect of the instant disclosure encompasses a kit for generating an epigenetically modified plant, plant part, or plant cell. The kit comprises one or more expression constructs expressing an engineered protein, one or more vectors comprising the constructs, or any combination thereof. The kit can also comprise one or more plants, plant parts, plant cell culture, or plant cells comprising the one or more expression constructs, one or more vectors, or any combination thereof.

### BRIEF DESCRIPTION OF THE FIGURES

FIG. 1A depicts a schematic of a generalized targeted methylation system comprising two molecules: a DNA targeting system and a DNA methylation protein. The DNA binding and methylation reagents may be connected via a direct fusion or engineered to interact in vivo through a system such as the SunTag system.

FIG. 1B is a schematic diagram of an example of methylation applied to a DNA sequence that subsequently blocks binding of a pathogen effector molecule, in this case the Xanthomonas effector protein TAL20 that induces expression of the cassava MeSWEET10a gene.

FIG. 1C depicts a plot showing the level of methylation targeted to the MeSWEET10a promoter by a DMS3-ZF fusion construct. Wildtype controls show no methylation across this sequence.

FIG. 2. An electrophoresis blot of an EMSA assay showing TAL20 binding to MeSWEET promoter sequence and inhibition of binding by DNA methylation. Lane 1, biotin labeled MeSWEET10a promoter sequence (EBE). Lane 2, addition of purified TAL20 protein results in gel shift. Lane 3, methylated EBE is bound less strongly than unmethylated EBE. Lanes 4-7, different competition experiments to further demonstrate inhibition of binding by methylation.

FIG. 3A. DMS3-ZF expression results in CpG methylation at the MeSWEET10a promoter EBE in vivo. Expression of transgenes in individual plants from two independent DMS3-expressing transgenic lines (133 and 204) as well as a ZF-only negative control line (216). Cassava variety names (60444 or TME 419) for each sample is shown above the lanes. First two rows: representative western blots (anti-FLAG) showing expression of the ZF (ZF-3×FLAG) protein with (top) and without (middle) DMS3. Relevant size standards are shown to the right (kD). Bottom: Coomassie Brilliant Blue stained Rubisco large subunit, loading control.

FIG. 3B. DMS3-ZF expression results in CpG methylation at the MeSWEET10a promoter EBE in vivo. Representative PCR-based bisulfite sequencing (ampBS-seq) results from samples shown in FIG. 3A. Top: Graphical depiction of MeSWEET10a promoter region assessed for methylation. The EBE (grey), a presumed TATA box (blue), and the ZF binding site (orange) are indicated. The predicted 5′ UTR and MeSWEET10a transcriptional start site are shown in green. The area within the dotted lined box (233 bp) was subjected to ampBS-seq. Bottom: CpG, CHG, and CHH DNA methylation levels (percent, y-axis) of the MeSWEET10a promoter (EBE, grey) measured by ampBS-seq with and without DMS3-ZF. Cassava variety names are indicated to the right. Methylation was called for cytosines with greater than 500 reads mapped.

FIG. 3C. DMS3-ZF expression results in CpG methylation at the MeSWEET10a promoter EBE in vivo. Representative wild-type (TME419) plant. Scale bar=14 cm.

FIG. 3D. DMS3-ZF expression results in CpG methylation at the MeSWEET10a promoter EBE in vivo. Representative DMS3-ZF-expressing (line #133) plant. Scale bar=14 cm.

FIG. 4A-C. Plot showing the level of methylation at the binding site of TAL20 (grey) using DMS3-ZF. Percent methylation is shown on the y-axis and sequence of the targeted region is shown on the x-axis. Cell line numbers are given to the right of the graphs. The colors of the bars in the graphs indicate the context of the methylated cytosines (legend to left of graphs).

FIG. 5. Disease phenotypes of leaves from plants transformed with DMS3-ZF directing methylation to the binding site of TAL20. A diagram of the experimental set up is shown on the left. Top right panel shows a photograph of a leaf from a plant transformed with DMS3-ZF directing methylation to the binding site of TAL20 (Methylated). Bottom right panel shows a photograph of a wild-type (WT) leaf infected with a Xam. Leaf lobes are labeled with X (WT Xam-infected), T (TAL20 mutant Xam) or M (mock-inoculated samples). The arrow indicates the presence (bottom) or absence (top) of water-soaking symptoms. Watersoaking is one of the earliest indicators of successful CBB infection by Xam.

FIG. 6A. Effect of ZF-directed methylation on CBB disease phenotypes in cassava. Plot showing the normalized relative expression of MeSweet10a in wild type and transgenic cassava plants expressing DMS3-ZF or ZF-only negative controls as determined by RT-qPCR. The cassava genes GTPb (Manes.09G086600) and PP2A4 (Manes.09G039900) were used as internal controls. Boxes are colored according to Xanthomonas treatment. Biological replicates (black dots) included in each background (x-axis) are as follows: n=4, 9, 4, 5, 10. The n included in each treatment group for each background are consistent. Welch's t-test p-values are noted above brackets within plot.

FIG. 6B. Effect of ZF-directed methylation on CBB disease phenotypes in cassava. Representative images of water-soaking phenotype of leaves from TME 419 WT and DMS3-ZF-expressing plants. Images were taken 4 days post-infection with either Xam668 (XamWT) or a Xam668 TAL20 deletion mutant (XamATAL20). Scale bar=0.5 cm. (C) Observed area (pixels, y-axis) of water-soaking from images of Xam-infiltrated leaves (genetic backgrounds, x-axis) 4 days post-infiltration. Calculated p-values (Kolmogorov-Smirnov test) are shown above brackets within plot. (D) Intensity of water-soaking phenotype (y-axis) of region measured in panel C. The negative mean grey-scale value for the water-soaked region relative to the average of the mock-treated samples within the same leaf is reported. Calculated p values (Kolmogorov-Smirnov test) are shown above brackets within plot. Box plots: Biological replicate values are indicated by dots. Horizontal black line within boxes indicates the value of the median while the box limits indicate the 25th and 75th percentiles as determined by R software; whiskers extend 1.5 times the interquartile range (1.5×IQR) from the 25th and 75th percentiles.

FIG. 6C. Effect of ZF-directed methylation on CBB disease phenotypes in cassava. Plot showing the observed area (pixels, y-axis) of water-soaking from images of Xam-infiltrated leaves (genetic backgrounds, x-axis) 4 days post-infiltration. Calculated p-values (Kolmogorov-Smirnov test) are shown above brackets within plot.

FIG. 6D. Effect of ZF-directed methylation on CBB disease phenotypes in cassava. Intensity of water-soaking phenotype (y-axis) of region measured in FIG. 6C. The negative mean grey-scale value for the water-soaked region relative to the average of the mock-treated samples within the same leaf is reported. Calculated p values (Kolmogorov-Smirnov test) are shown above brackets within plot. Box plots: Biological replicate values are indicated by dots. Horizontal black line within boxes indicates the value of the median while the box limits indicate the 25th and 75th percentiles as determined by R software; whiskers extend 1.5 times the interquartile range (1.5×IQR) from the 25th and 75th percentiles.

FIG. 7A-C. Methylation at the binding site of TAL20 (grey) using SunTag-DRM. Top: schematic diagram of the promoter of MeSWEET10a showing the approximate binding sites of gRNA4 and gRNA5. Bottom: level of methylation in transformed plant lines. Percent methylation is shown on the y-axis and sequence of the targeted region is shown on the x-axis. Line numbers are given to the right of the graphs. The color of the bars in the graphs indicate the context of the methylated cytosines (legend to left of graphs). SunTag-DRM_noNLS gRNAs 4+5: SunTag-DRM with no nuclear localization system (NLS) and gRNA 4+gRNA 5 guide RNAs. SunTag-DRM_noNLS gRNA 5: SunTag-DRM with no nuclear localization system (NLS) a gRNA 5 guide RNA. SunTag-DRM_noNLS gRNA 4: SunTag-DRM with no nuclear localization system (NLS) a gRNA 4 guide RNA.

FIG. 8A. Effect of CRISPR-targeted methylation on CBB disease phenotypes in cassava. Methylation at the binding site of TAL20 (grey) using SunTag-DRM. Top: schematic diagram of the promoter of MeSWEET10a. Bottom: level of methylation in transformed plant lines. Percent methylation is shown on the y-axis and sequence of the targeted region is shown on the x-axis. Line numbers are given to the right of the graphs. The color of the bars in the graphs indicate the context of the methylated cytosines (legend to left of graphs).

FIG. 8B. Effect of CRISPR-targeted methylation on CBB disease phenotypes in cassava. Normalized MeSWEET10a expression (y-axis, Log10 scale) in WT and transgenic lines as determined by RT-qPCR. The cassava genes GTPb (Manes.09G086600) and PP2A4 (Manes.09G039900) were used as internal controls. MeSWEET10a expression is normalized to WT TME 419-Xam-treated samples. Boxes are colored according to Xanthomonas treatment. Biological replicates (black dots) included in each background (x-axis) are as follows: n=3, 6, 6, 2, 6. The n included in each treatment group for each background are consistent. Horizontal black line within boxes indicates the value of the median while the box limits indicate the 25th and 75th percentiles as determined by R software; whiskers extend 1.5 times the interquartile range (1.5×IQR) from the 25th and 75th percentiles.

FIG. 9A-B. Methylation of nCBP1 promoter region using SunTag-DRM. Percent methylation is shown on the y-axis and sequence of the targeted region is shown on the x-axis. Lines transformed with the construct containing no guide RNAs and wild type (WT) are shown as negative controls. Line numbers are given to the right of the graphs. The color of the bars in the graphs indicate the context of the methylated cytosines (legend to left of graphs). Top: schematic diagram of the promoter of nCBP1 showing the approximate binding sites of the gRNAs.

FIG. 10A-B. Methylation of nCBP2 promoter region using SunTag-DRM. Percent methylation is shown on the y-axis and sequence of the targeted region is shown on the x-axis. Lines transformed with the construct containing no guide RNAs and wild type (WT) are shown as negative controls. Line numbers are given to the right of the graphs. The color of the bars in the graphs indicate the context of the methylated cytosines (legend to left of graphs). Top: schematic diagram of the promoter of nCBP1 showing the approximate binding sites of the gRNAs.

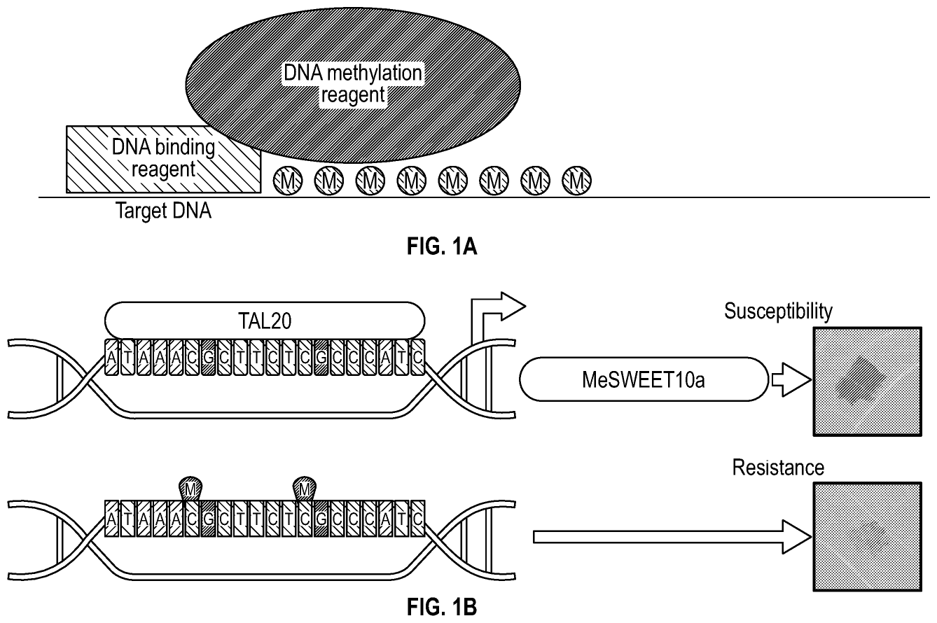

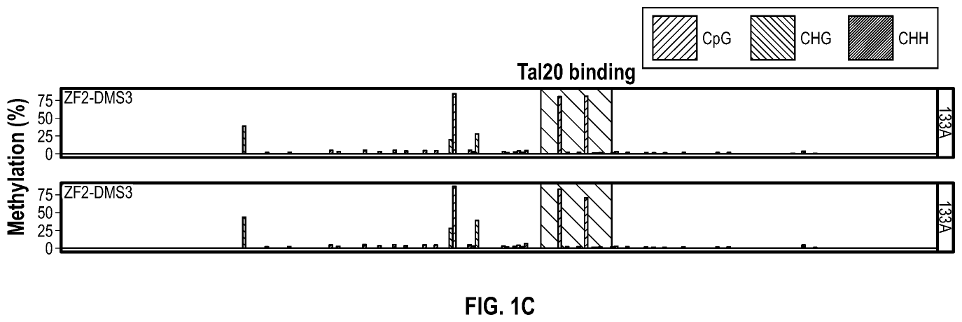

### DETAILED DESCRIPTION

The present disclosure encompasses engineered proteins for methylating a target nucleic acid sequence in a plant pathogen susceptibility gene, expression constructs expressing the engineered proteins, and methods of using the expression constructs to improve or provide disease resistance to a plant. The method comprises improving disease resistance using epigenetic modification to regulate the expression of plant susceptibility genes. More specifically, the disclosure is directed to targeted DNA methylation of specific DNA loci in a plant to modulate the activity of susceptibility genes to thereby improve or provide disease resistance to the plant. The methods can provide robust and selective modulation of genes associated with plant defense responses. Importantly, a useful quality of DNA methylation is that, once established, it can be inherited faithfully in the absence of the original trigger that initially caused methylation, much like changes to the sequence of DNA. Advantageously, the resulting plants are not subject to the same cumbersome regulatory hurdles as more traditionally genetically modified crops.

The engineered proteins and methods can provide a high level of specificity, essentially only methylating a targeted locus, thereby preventing off target methylation that may affect plant growth and development. Surprisingly and unexpectedly, the engineered proteins and methods can co-target multiple methylation polypeptides or multiple copies of methylation polypeptides to one or more loci, can simultaneously methylate more than one targeted methylation locus, and can regulate the expression of multiple genes simultaneously. Further, expression of components of the system under the control of regulated and tissue-specific promoters can provide additional fine-tuning of gene expression. Combined, these qualities can provide precise tunable control of specific susceptibility genes in specific tissues to create partial or complete reduction of gene expression states rather than complete loss-of-function, as many defense genes are often critical for normal plant growth and development. Further, the engineered proteins and methods of the instant disclosure are widely applicable to diverse plants and diseases, even among distantly related dicot and monocot plants like cassava and maize. Accordingly, an engineered protein engineered to modulate the expression of one gene can be used to modulate the expression of that gene in diverse plant species.

### I. Engineered Protein

One aspect of the present disclosure encompasses an engineered protein for methylating a target nucleic acid sequence in a plant pathogen susceptibility gene. The engineered protein comprises a methylation polypeptide linked to a targeting polypeptide, wherein the targeting polypeptide is engineered to bind a target DNA sequence in a target methylation locus in a plant pathogen susceptibility gene. Binding of the DNA binding domain of the engineered protein to the target DNA sequence targets the engineered protein to the target locus, thereby mediating methylation of one or more methylation sites in the target locus. Methylating the one or more methylation sites in the target locus modulates the expression of the plant pathogen susceptibility gene. A plant comprising the one or more plant susceptibility genes having modified expression has improved resistance to a plant pathogen.

### (a) Susceptibility Genes

The engineered proteins of the instant disclosure can modify the expression of one or more susceptibility genes. As used herein, the term “susceptibility genes” or “plant pathogen susceptibility gene” are used interchangeably and refer to any gene, the increased or decreased expression of which in a plant increases disease resistance of the plant against a pathogen. Non-limiting examples of pathogens include viral, bacterial, oomycete, animal such as pathogenic nematodes, or fungal pathogens, or any combinations thereof. Susceptibility genes can be any gene capable of contributing to one or more plant mechanisms associated with resistance and susceptibility of a plant to a pathogen. Such genes are known in the art, or can be identified using methods and tools known to individuals of skill in the art. Individuals of skill in the art will also recognize that susceptibility genes can be conserved across plant species. Non-limiting examples of susceptibility genes are shown in Table 1.

TABLE 1
Non-limiting examples of susceptibility genes

| | | Pathogen | | Proposed | |
|---|---|---|---|---|---|
| Pathogen | Host | protein | Host protein | mechanism | References |
| Xanthomonas | cassava | TAL20 | MeSWEET10a | Sugar | 10.1094/MPMI-06-14-0161-R |
| axonopodis pv. | | | | transport to | |
| manihotis | | | | apoplast | |
| X. oryzae pv. | rice | TAL | Several | Sugar | https://doi.org/10.1111/nph.12411 |
| oryzae | | effectors | SWEET genes | transport to | |
| | | | | apoplast | |
| Xanthomonas | Citrus | PthA4 | CsLOB1 | N/A | https://pubmed.ncbi.nlm.nih.gov/28371200/ |
| citri subsp. citri | | | | | |
| TuMV | Arabidopsis | NA | elF(iso)4E | Protein | 10.1016/s0960-9822(02)00898-9 |
| | | | | translation | |
| CBSV and | cassava | NA | nCBP1/nCBP2 | Protein | doi.org/10.1111/pbi. 12987 |
| UCBSV | | | | translation | |
| Cucumber vein | Cucumber | N/A | elF(iso)4E | Protein | https://pubmed.ncbi.nlm.nih.gov/26808139/ |
| yellowing virus | | | | translation | |
| (Ipomovirus), | | | | | |
| Zucchini yellow | | | | | |
| mosaic virus | | | | | |
| and Papaya | | | | | |
| ring spot | | | | | |
| mosaic virus-W | | | | | |
| Powdery | Tomato | N/A | Mlo | N/A | https://www.nature.com/articles/s41598-017-00578-x |
| mildew fungal | | | | | |
| pathogen | | | | | |
| Oidium | | | | | |
| neolycopersici | | | | | |
| powdery | Wheat | N/A | Edr1 | N/A | https://pubmed.ncbi.nlm.nih.gov/28502081/ |
| mildew, | | | | | |
| Blumeria | | | | | |
| graminis f. sp. | | | | | |
| tritici | | | | | |

In some aspects, a susceptibility gene is a gene, the reduced expression of which increases disease resistance of the plant and is referred to hereinafter as a pathogen susceptibility gene. Disease in plants arises from a compatible interaction between plant and pathogen. Most plant pathogens reprogram host gene expression patterns to directly benefit the pathogen. Reprogrammed genes required for pathogen survival and proliferation can be thought to depend on the expression of pathogen-specific susceptibility genes termed S genes. Hence, reducing the expression of a plant S gene that critically facilitates the infection process of a pathogen or supports compatibility with the pathogen with its host increases resistance of the plant against the pathogen.

Non-limiting examples of S genes include genes having transcription activator-like (TAL) effector (TALE) binding sites in the promoter. TALE proteins (TALEs) are secreted by Xanthomonas bacteria when they infect various plant species. Similar proteins can be found in the pathogenic bacterium Ralstonia solanacearum and Burkholderia rhizoxinica. The term TALE-like protein is used herein to refer to the putative protein family encompassing the TALEs and related proteins. These proteins can bind promoter sequences in the host plant and activate the expression of plant genes that aid bacterial infection. Other susceptibility genes include mutant inactivated genes that normally provide resistance to pathogens, including inactivated genes encoding pectate lyases, the MLO gene, the Lr34 gene, translation elongation initiation factor genes such as eIF4E and eIF4G, and the TALE protein targets Os8N3 (aka. Xa13 and OsSWEET11), Os11N3 (aka. OsSWEET14) induced by Xanthomonas species.

A non-limiting example of pathogenesis in plants includes the susceptibility of cassava to cassava brown streak disease virus (CBSV). Susceptibility to CBSV is facilitated by expression of at least the nCBP-1 and nCBP-2 S genes within the eIF4E family. Accordingly, disease resistance to CBSV in cassava can be improved by methylation-induced reduction of expression of the nCBP-1 and nCBP-2 S genes, and combinations thereof. In another example, susceptibility of cassava to cassava bacterial blight (CBB) is facilitated by at least the MeSWEET10a S gene and pectate lyase genes (cassava4.1_007568 and cassava4.1_007516) that also contribute to disease severity. Accordingly, disease resistance to CBB in cassava can be improved by methylating-induced reduction of expression of MeSWEET10a, pectate lyase genes, and combinations thereof. In some aspects, the susceptibility gene is MeSWEET10a. In other aspects, the susceptibility gene is nCBP-1, nCBP-2, or combinations thereof. In some aspects, the susceptibility gene is nCBP-1 and nCBP-2. In some aspects, the plant is cassava, the susceptibility gene is nCBP-1 and nCBP-2, and the pathogen is a viral pathogen that causes cassava brown streak disease.

In some aspects, a susceptibility gene is any gene, the increased expression of which increases disease resistance of the plant (referred to hereinafter as “resistance genes”). Plant resistance mechanisms include pre-formed structures and chemicals, and infection-induced responses of the immune system. For instance, the resistance gene can be a gene that contributes to the cuticle, cell walls, and reinforcement of cell walls and the cuticle, or a gene that contributes to the production of antimicrobial compounds such as antimicrobial chemicals (for example: polyphenols, sesquiterpene lactones, saponins, hydrogen peroxide or peroxynitrite, or more complex phytoalexins such as genistein or camalexin), antimicrobial peptides, enzyme inhibitors, detoxifying enzymes that break down pathogen-derived toxins, antimicrobial proteins such as defensins, thionins, or PR-1, antimicrobial enzymes such as chitinases, beta-glucanases, or peroxidases, the hypersensitivity response, or receptors that perceive pathogen presence and activate inducible plant defenses, among others. Non-limiting examples of disease resistance genes include pattern recognition receptor (PRR) genes, R (resistance) genes whose products mediate resistance to a specific virus, bacterium, oomycete, fungus, nematode or insect strain, pectate lyase genes, mutant susceptibility gene alleles that prevent pathogens from reprogramming genes required for pathogen survival and proliferation, resistance genes triggered by TALE proteins such as the Os-8N3 gene, the XA13 gene, the MLO gene, the Lr34 gene, translation elongation initiation factor genes such as eif4e and eif4g, and the xa13 gene, and any combination thereof.

### (b) Methylation Polypeptides

The engineered protein of the instant disclosure comprises a methylation polypeptide linked to a targeting polypeptide. The methylation polypeptide comprises a DNA methylation domain of a DNA methylation protein. A DNA methylation domain comprises an amino acid sequence having about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similarity to a methylation protein, portion of a methylation protein, or a polypeptide derived from a methylation protein capable of mediating methylation or de-methylation of one or more methylation sites at a target methylation locus. The methylation protein, portion of a methylation protein, or a polypeptide derived from a methylation protein is hereinafter referred to as a methylation protein. A target methylation locus can be any nucleic acid sequence of any size comprising one or more methylation sites which, when methylated or de-methylated, can modulate the activity of a nucleic acid sequence.

DNA methylation is a biological process by which methyl groups are added to methylation sites in DNA molecule. Methylation of one or more nucleic acid can change the activity of a nucleic acid sequence without changing the sequence. Two of DNA's four bases, cytosine and adenine, can be methylated. Cytosine methylation is widespread in both eukaryotes and prokaryotes. In plants, DNA methylation is found in three different sequence contexts: CG (or CpG), CHG (or CpHpG), or CHH (or CpHpH), where H corresponds to A, T or C. Accordingly, the cytosine can be methylated at CpG, CpHpG, and CpHpH methylation sites, where H represents any nucleotide except guanine. Individuals of skill in the art will recognize that other methylation sequence contexts may exit.

In plants, DNA methylation is established by the DNA methyltransferase enzyme DOMAINS REARRANGED METHYLTRANSFERASE 2 (DRM2), which is targeted to the genome by 24-nucleotide small interfering RNAs (siRNAs) through a pathway termed RNA-directed DNA methylation (RdDM). This pathway also requires two plant-specific RNA polymerases: Pol-IV, which functions to transcribe DNA to initiate siRNA biogenesis, and Pol-V, which functions to generate scaffold transcripts that recruit downstream RdDM factors including DRM2. The currently accepted view is that RNA-directed DNA methylation occurs in the genome wherever Pol IV and Pol V are simultaneously transcribing the same area of chromatin. To date, the following proteins of the RdDM pathway have been identified: SHH1 SUVH2 and SUVH9 which act as recruitment factors for Pol IV and Pol V, DMS3, NRPE1 (largest subunit of Pol V), NRPD1 (largest subunit of Pol IV), CLSY1, NRPD2, RDR2, DCL3, AGO4, DRD1, RDM1, DMS4, KTF1, IDN2, and SUVR2. It will be recognized that other pathways of DNA methylation and methylation proteins could be identified in the future and are also included in this disclosure.

Once methylation is established at a location in the genome, it can often be efficiently maintained and can be faithfully inherited in subsequent plant generations, even after removal of the original trigger that initially caused methylation, thereby providing resistance in the subsequent generations. For instance, the RNA-directed DNA methylation is a self-reinforcing maintenance loop because Pol IV and Pol V are attracted to chromatin by the very marks that they are responsible for targeting in the first place. In addition, two other maintenance methylation systems, the CG/MET1 system and the CMT3/CMT2 system, are recruited to sites of established RdDM and further maintain DNA methylation. Hence, the disclosure encompasses modification of genes of the maintenance methylation systems such as the CG/MET1 system, the CMT3/CMT2 system, or combinations thereof.

Accordingly, a methylation protein as used herein refers to any one or more proteins associated with the RdDM pathway, any one or more proteins associated with removing any obstacles to methylation, any one or more proteins of the maintenance methylation systems, or combinations thereof.

The methylation protein can also be a host or exogenous protein capable of contributing to methylation of a locus in the host plant. For instance, the methylation protein can be a plant methylation protein derived from the host, as well as from other plants, or can also be a microbial or animal methylation protein. For instance, the methylation protein can be a bacterial CG-specific Sssl methyltransferase such as MQ1.

In some aspects, the engineered protein comprises a DNA methylation domain of a DMS3 protein. In some aspects, the DMS3 protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 2. In some aspects, the DMS3 protein is encoded by a nucleic acid sequence comprising at least about 75% or more, at least about 85% or more, at least about 95% or more, or 100% sequence identity with a nucleic acid sequence of SEQ ID NO: 2.

In some aspects, the engineered protein comprises a DNA methylation domain of a DRM2 protein. In some aspects, the DRM2 protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 7. In some aspects, the DRM2 protein is encoded by a nucleic acid sequence comprising at least about 75% or more, at least about 85% or more, at least about 95% or more, or 100% sequence identity with a nucleic acid sequence of SEQ ID NO: 7.

In some aspects, the engineered protein comprises a DNA methylation domain of a MQ1 protein. In some aspects, the MQ1 protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 6. In some aspects, the MQ1 protein is encoded by a nucleic acid sequence comprising at least about 75% or more, at least about 85% or more, at least about 95% or more, or 100% sequence identity with a nucleic acid sequence of SEQ ID NO: 6.

In some aspects, methylation polypeptides comprise DMS3 and NRPD1, and the methylation polypeptides are co-targeted with H3K4me3 removal. In some aspects, the one or more methylation polypeptides comprise a protein within the eIF4E family such as nCBP-1 and nCBP-2. In some aspects, the one or more methylation polypeptides comprise the bacterial CG-specific Sssl methyltransferase MQ1. In some aspects, the one or more methylation polypeptides comprise Sssl, DMS3, and NRPD1.

Modulating methylation of methylation sites in a target methylation locus in a susceptibility gene modulates expression of the susceptibility gene. In many instances, modulation of DNA methylation occurs in promoter regions of a gene. However, methylation sites can also be found in the body of the gene. Accordingly, the target methylation locus can be in a coding region of a susceptibility gene or can be in a non-coding region in the genome which, when methylated or demethylated, is capable of modifying expression of the gene.

Modulating methylation of the target locus can modulate expression of the gene by reducing or improving the binding ability of a transcriptional factor to a promoter region of the gene. For instance, modulating methylation of the target locus can modulate expression of the gene by physically impeding or aiding the binding of transcriptional proteins to the target locus in a promoter region of the gene to thereby modulate the expression of the gene. For instance, a TALE protein can be prevented from binding the promoter of a given S gene by methylating the binding site of the TALE protein in the promoter region of the S gene, thereby impairing the pathogen's ability to alter host gene expression to its benefit, and thereby decreasing susceptibility to the pathogen. DNA methylation can also modulate the expression of the gene by inducing chromatin remodeling at the promoter that can affect expression of the gene. Methylated DNA can be bound by proteins known as methyl-CpG-binding domain proteins (MBDs), which then recruit additional proteins to the locus, such as histone modification proteins and other chromatin remodeling proteins, thereby either forming compact, inactive chromatin, termed heterochromatin to inhibit expression of the gene, or forming euchromatin (loose chromatin structure) to induce expression of the gene. Further, in some instances, strong gene expression is antagonistic to DNA methylation. Accordingly, by simultaneously recruiting a transcriptional repressor, together with a methylation targeting effector, the antagonistic force of expression can be reduced to more potently establish high levels of DNA methylation and gene silencing.

By similar mechanisms, DNA methylation in the body of the gene can affect expression of the gene by, e.g., regulating splicing, suppressing or inducing the activity of intragenic transcriptional units (cryptic promoters or transposable elements), preventing or inducing the activation of cryptic start sites, among others.

### (c) Targeting Polypeptide

The engineered protein of the instant disclosure comprises a methylation polypeptide linked to a targeting polypeptide. The targeting polypeptide comprises a sequence-specific DNA binding domain, wherein the DNA binding domain binds a target DNA sequence in a polynucleotide encoding a plant pathogen susceptibility gene. The targeting polypeptide is capable of targeting one or more methylation polypeptides of the instant disclosure to a target methylation locus in a polynucleotide encoding a plant pathogen susceptibility gene.

Targeting polypeptides are linked to the methylation polypeptide to target the engineered protein, including the methylation polypeptide, to the target methylation locus. Multiple useful methods of linking proteins are known in the art and included herein. For instance, the targeting polypeptide can be fused to the methylation polypeptides. The targeting polypeptide can be fused to the methylation polypeptides by at least one linker, such as a peptide linker. The linker can be flexible (e.g., comprising small, non-polar (e.g., Gly) or polar (e.g., Ser, Thr) amino acids). Examples of suitable linkers are well known in the art, and programs to design linkers are readily available (Crasto et al., Protein Eng., 2000, 13 (5): 3096-312), the disclosure of which is incorporated herein in its entirety.

The targeting polypeptide can also be indirectly linked to the methylation polypeptide such as through linking moieties in the targeting polypeptide or the methylation polypeptide, including but not limited to, antibodies, antibody fragments, peptides, small molecules, polysaccharides, nucleic acids, aptamers, peptidomimetics and other mimetics, a ligand, a ligand fragment, a receptor, a receptor fragment, a polypeptide, a peptide, a coenzyme, a coregulator, alone or in combination. These moieties may be utilized to specifically link the targeting polypeptide and the methylation polypeptide. For instance, the methylation polypeptide and the targeting polypeptide can be linked through a purification tag and/or an epitope tag. Exemplary tags include, but are not limited to, glutathione-S-transferase (GST), chitin binding protein (CBP), maltose binding protein, thioredoxin (TRX), poly (NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, biotin carboxyl carrier protein (BCCP), and calmodulin.

A targeting polypeptide comprises a targeting domain. The targeting domain comprises an amino acid sequence which can specifically recognize and directly bind a nucleic acid sequence in the target methylation locus in nucleic acid sequences encoding a susceptibility gene. Alternatively, the targeting domain can have affinity to a protein that specifically recognizes and binds the nucleic acid sequence to thereby indirectly bind the nucleic acid sequence. The nucleic acid sequence can be within or adjacent to the target methylation locus, or can be distantly located from the target methylation locus, provided that binding of the targeting domain to the nucleic acid sequence brings the targeting polypeptide and linked methylation polypeptide in proximity to the target methylation locus to mediate methylation of the target methylation locus.

The term “targeting domain” as used herein refers to any amino acid sequence derived from a targeting protein or system wherein the targeting domain has about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similarity to a targeting protein or system, portion of a targeting protein or system, or polypeptides derived from a targeting protein or system. The targeting protein can be a host or exogenous protein with innate ability to bind a nucleic acid sequence in a methylation locus to target the targeting polypeptide to the target methylation locus. Alternatively, the targeting protein can be a programmable targeting protein engineered to bind a nucleic acid sequence in a target methylation locus. A targeting protein can be any single or group of components capable of targeting components of the engineered system to a target methylation locus.

As explained above in Section I(b), multiple copies of a methylation polypeptide, more than one methylation polypeptide, or a combination of multiple copies of a methylation polypeptide and more than one methylation polypeptide, can be targeted to a locus or to one or more loci. Accordingly, a system of the instant disclosure can include multiple targeting polypeptides each engineered to target a methylation polypeptide to the target locus or loci. Alternatively, a system of the instant disclosure can include one or more targeting polypeptides, each engineered to target multiple copies of a methylation polypeptide or more than one methylation polypeptide to the target locus.

A programmable targeting protein can be any single or group of components capable of targeting engineered protein to a target nucleic acid sequence to mediate methylation of methylation sites at a target methylation locus. The target methylation locus can be in a coding or regulatory region of interest or can be in any other location in a nucleic acid sequence of interest. A gene can be a protein-coding gene, an RNA coding gene, or an intergenic region. The target locus can be in a nuclear, organellar, or extrachromosomal nucleic acid sequence. The cell can be a eukaryotic cell. In some aspects, the cell is a plant cell. In some aspects, the plant is a cassava plant.

As used herein, a programmable targeting protein generally comprises a programmable, sequence-specific DNA-binding domain of a programmable nucleic acid editing system. Such editing systems can be engineered to edit specific DNA or RNA sequences to repress transcription or translation of an mRNA encoded by the gene, and/or produce mutant proteins with reduced activity or stability. Non-limiting examples of programmable polynucleotide targeting nucleases include, without limit, an RNA-guided clustered regularly interspersed short palindromic repeats (CRISPR)/CRISPR-associated (Cas) (CRISPR/Cas) nuclease system, a CRISPR/Cpf1 nuclease system, a zinc finger nuclease (ZFN), a transcription activator-like effector nuclease (TALEN), a meganuclease, a ribozyme, or a programmable DNA binding domain linked to a nuclease domain. Other suitable programmable polynucleotide targeting nucleases will be recognized by individuals skilled in the art. Such systems rely for specificity on the delivery of exogenous protein(s), and/or a guide RNA (gRNA) or single guide RNA (sgRNA) having a sequence which binds specifically to a gene sequence of interest. When the programmable polynucleotide targeting nuclease comprises more than one component, such as a protein and a guide nucleic acid, the multi-component modification system can be modular, in that the different components may optionally be distributed among two or more nucleic acid constructs as described herein. The components can be delivered by a plasmid or viral vector or as a synthetic oligonucleotide. More detailed descriptions of programmable nucleic acid editing system can be as described further below.

The programmable nucleic acid-binding domain may be designed or engineered to recognize and bind different nucleic acid sequences. In some aspects, the nucleic acid-binding domain is mediated by interaction between a protein and the target nucleic acid sequence. Thus, the nucleic acid-binding domain may be programmed to bind a nucleic acid sequence of interest by protein engineering. Methods of programming a nucleic acid domain are well recognized in the art.

In other targeting nucleases, the nucleic acid-binding domain is mediated by a guide nucleic acid that interacts with a protein of the targeting domain and the target nucleic acid sequence. In such instances, the programmable nucleic acid-binding domain may be targeted to a nucleic acid sequence of interest by designing the appropriate guide nucleic acid. Methods of designing guide nucleic acids are recognized in the art when provided with a target sequence using available tools that are capable of designing functional guide nucleic acids. It will be recognized that gRNA sequences and design of guide nucleic acids can and will vary at least depending on the particular nuclease used. By way of non-limiting example, guide nucleic acids optimized by sequence for use with a Cas9 nuclease, are likely to differ from guide nucleic acids optimized for use with a CPF1 nuclease, though it is also recognized that the target site location is a key factor in determining guide RNA sequences.

When a targeting nuclease comprises more than one component, such as a protein and a guide nucleic acid, the multi-component targeting nuclease can be modular, in that the different components may optionally be distributed among two or more nucleic acid constructs as described herein.

In some aspects, a targeting protein is a CRISPR system. Accordingly, in some aspects, the targeting polypeptide comprises one or more domains encoding a CRISPR targeting system. In other aspects, a targeting protein is an Argonaute system. Accordingly, in some aspects, the targeting polypeptide comprises one or more domains encoding an Argonaute targeting system. In yet other aspects, a targeting protein is a zinc finger DNA binding domain. Accordingly, in some aspects, the targeting polypeptide comprises a zinc finger DNA binding domain. In additional aspects, a targeting protein is a TALE protein. Accordingly, in some aspects, the targeting polypeptide comprises a TALE protein. In further aspects, a targeting protein is a DNA binding domain of a meganuclease. Accordingly, in some aspects, the targeting polypeptide comprises a meganuclease. In some aspects, a targeting protein is a DNA binding domain of a rare-cutting endonuclease system. Accordingly, in some aspects, the targeting polypeptide comprises a DNA binding domain of a rare-cutting endonuclease system.

In some aspects, the programmable targeting protein is a CRISPR/Cas nuclease system comprising a nuclease and a guide RNA (gRNA). In some aspects, the targeting protein comprises a nuclease-deficient CAS9 protein (dCAS9).

i. CRISPR Nuclease Systems.

The programmable targeting nuclease can be an RNA-guided CRISPR endonuclease system. The CRISPR system comprises a guide RNA or sgRNA to a target sequence at which a protein of the system introduces a double-stranded break in a target nucleic acid sequence, and a CRISPR-associated endonuclease. The gRNA is a short synthetic RNA comprising a sequence necessary for endonuclease binding, and a preselected ˜20 nucleotide spacer sequence targeting the sequence of interest in a genomic target. Non-limiting examples of endonucleases include Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a homolog thereof, a recombination of the naturally occurring molecule thereof, a codon-optimized version thereof, or a modified version thereof, or any combination thereof.

The CRISPR nuclease system may be derived from any type of CRISPR system, including a type I (i.e., IA, IB, IC, ID, IE, or IF), type II (i.e., IIA, IIB, or IIC), type III (i.e., IIIA or IIIB), or type V CRISPR system. The CRISPR/Cas system may be from Streptococcus sp. (e.g., Streptococcus pyogenes), Campylobacter sp. (e.g., Campylobacter jejuni), Francisella sp. (e.g., Francisella novicida), Acaryochloris sp., Acetohalobium sp., Acidaminococcus sp., Acidithiobacillus sp., Alicyclobacillus sp., Allochromatium sp., Ammonifex sp., Anabaena sp., Arthrospira sp., Bacillus sp., Burkholderiales sp., Caldicelulosiruptor sp., Candidatus sp., Clostridium sp., Crocosphaera sp., Cyanothece sp., Exiguobacterium sp., Finegoldia sp., Ktedonobacter sp., Lactobacillus sp., Lyngbya sp., Marinobacter sp., Methanohalobium sp., Microscilla sp., Microcoleus sp., Microcystis sp., Natranaerobius sp., Neisseria sp., Nitrosococcus sp., Nocardiopsis sp., Nodularia sp., Nostoc sp., Oscillatoria sp., Polaromonas sp., Pelotomaculum sp., Pseudoalteromonas sp., Petrotoga sp., Prevotella sp., Staphylococcus sp., Streptomyces sp., Streptosporangium sp., Synechococcus sp., or Thermosipho sp.

Non-limiting examples of suitable CRISPR systems include CRISPR/Cas systems, CRISPR/Cpf systems, CRISPR/Cmr systems, CRISPR/Csa systems, CRISPR/Csb systems, CRISPR/Csc systems, CRISPR/Cse systems, CRISPR/Csf systems, CRISPR/Csm systems, CRISPR/Csn systems, CRISPR/Csx systems, CRISPR/Csy systems, CRISPR/Csz systems, and derivatives or variants thereof. Preferably, the CRISPR system may be a type II Cas9 protein, a type V Cpf1 protein, or a derivative thereof. In some aspects, the CRISPR/Cas nuclease is Streptococcus pyogenes Cas9 (SpCas9), Streptococcus thermophilus Cas9 (StCas9), Campylobacter jejuni Cas9 (CjCas9), Francisella novicida Cas9 (FnCas9), or Francisella novicida Cpf1 (FnCpf1).

In general, a protein of the CRISPR system comprises a RNA recognition and/or RNA binding domain, which interacts with the guide RNA. A protein of the CRISPR system also comprises at least one nuclease domain having endonuclease activity. For example, a Cas9 protein may comprise a RuvC-like nuclease domain and an HNH-like nuclease domain, and a Cpf1 protein may comprise a RuvC-like domain. A protein of the CRISPR system may also comprise DNA binding domains, helicase domains, RNase domains, protein-protein interaction domains, dimerization domains, as well as other domains.

A protein of the CRISPR system may be associated with guide RNAs (gRNA). The guide RNA may be a single guide RNA (i.e., sgRNA), or may comprise two RNA molecules (i.e., crRNA and tracrRNA). The guide RNA interacts with a protein of the CRISPR system to guide it to a target site in the DNA. The target site has no sequence limitation except that the sequence is bordered by a protospacer adjacent motif (PAM). For example, PAM sequences for Cas9 include 3′-NGG, 3′-NGGNG, 3′-NNAGAAW, and 3′-ACAY, and PAM sequences for Cpf1 include 5′-TTN (wherein N is defined as any nucleotide, W is defined as either A or T, and Y is defined as either C or T). Each gRNA comprises a sequence that is complementary to the target sequence (e.g., a Cas9 gRNA may comprise GN17-20GG). The gRNA may also comprise a scaffold sequence that forms a stem loop structure and a single-stranded region. The scaffold region may be the same in every gRNA. In some aspects, the gRNA may be a single molecule (i.e., sgRNA). In other aspects, the gRNA may be two separate molecules. Those skilled in the art are familiar with gRNA design and construction, e.g., gRNA design tools are available on the internet or from commercial sources.

A CRISPR system may comprise one or more nucleic acid binding domains associated with one or more, or two or more selected guide RNAs used to direct the CRISPR system to one or more, or two or more selected target methylation loci. For instance, a nucleic acid binding domain may be associated with one or more, or two or more selected guide RNAs, each selected guide RNA, when complexed with a nucleic acid binding domain, causing the CRISPR system to localize to the target of the guide RNA.

ii. CRISPR Nickase Systems.

The programmable targeting nuclease can also be a CRISPR nickase system. CRISPR nickase systems are similar to the CRISPR nuclease systems described above except that a CRISPR nuclease of the system is modified to cleave only one strand of a double-stranded nucleic acid sequence. Thus, a CRISPR nickase, in combination with a guide RNA of the system, may create a single-stranded break or nick in the target nucleic acid sequence. Alternatively, a CRISPR nickase in combination with a pair of offset gRNAs may create a double-stranded break in the nucleic acid sequence.

A CRISPR nuclease of the system may be converted to a nickase by one or more mutations and/or deletions. For example, a Cas9 nickase may comprise one or more mutations in one of the nuclease domains, wherein the one or more mutations may be D10A, E762A, and/or D986A in the RuvC-like domain, or the one or more mutations may be H840A (or H839A), N854A and/or N863A in the HNH-like domain.

iii. ssDNA-Guided Argonaute Systems.

Alternatively, the programmable targeting nuclease may comprise a single-stranded DNA-guided Argonaute endonuclease. Argonautes (Agos) are a family of endonucleases that use 5′-phosphorylated short single-stranded nucleic acids as guides to cleave nucleic acid targets. Some prokaryotic Agos use single-stranded guide DNAs and create double-stranded breaks in nucleic acid sequences. The ssDNA-guided Ago endonuclease may be associated with a single-stranded guide DNA.

The Ago endonuclease may be derived from Alistipes sp., Aquifex sp., Archaeoglobus sp., Bacteroides sp., Bradyrhizobium sp., Burkholderia sp., Cellvibrio sp., Chlorobium sp., Geobacter sp., Mariprofundus sp., Natronobacterium sp., Parabacteriodes sp., Parvularcula sp., Planctomyces sp., Pseudomonas sp., Pyrococcus sp., Thermus sp., or Xanthomonas sp. For instance, the Ago endonuclease may be Natronobacterium gregoryi Ago (NgAgo). Alternatively, the Ago endonuclease may be Thermus thermophilus Ago (TtAgo). The Ago endonuclease may also be Pyrococcus furiosus (PfAgo).

The single-stranded guide DNA (gDNA) of an ssDNA-guided Argonaute system is complementary to the target site in the nucleic acid sequence. The target site has no sequence limitations and does not require a PAM. The gDNA generally ranges in length from about 15-30 nucleotides. The gDNA may comprise a 5′ phosphate group. Those skilled in the art are familiar with ssDNA oligonucleotide design and construction.

iv. Zinc Finger Nucleases.

The programmable targeting nuclease may be a zinc finger nuclease (ZFN). A ZFN comprises a DNA-binding zinc finger region and a nuclease domain. The zinc finger region may comprise from about two to seven zinc fingers, for example, about four to six zinc fingers, wherein each zinc finger binds three nucleotides. The zinc finger region may be engineered to recognize and bind to any DNA sequence. Zinc finger design tools or algorithms are available on the internet or from commercial sources. The zinc fingers may be linked together using suitable linker sequences.

A ZFN also comprises a nuclease domain, which may be obtained from any endonuclease or exonuclease. Non-limiting examples of endonucleases from which a nuclease domain may be derived include, but are not limited to, restriction endonucleases and homing endonucleases. The nuclease domain may be derived from a type II-S restriction endonuclease. Type II-S endonucleases cleave DNA at sites that are typically several base pairs away from the recognition/binding site and, as such, have separable binding and cleavage domains. These enzymes generally are monomers that transiently associate to form dimers to cleave each strand of DNA at staggered locations. Non-limiting examples of suitable type II-S endonucleases include BfiI, BpmI, BsaI, BsgI, BsmBI, BsmI, BspMI, FokI, MboII, and SapI. The type II-S nuclease domain may be modified to facilitate dimerization of two different nuclease domains. For example, the cleavage domain of FokI may be modified by mutating certain amino acid residues. By way of non-limiting example, amino acid residues at positions 446, 447, 479, 483, 484, 486, 487, 490, 491, 496, 498, 499, 500, 531, 534, 537, and 538 of FokI nuclease domains are targets for modification. For example, one modified FokI domain may comprise Q486E, I499L, and/or N496D mutations, and the other modified FokI domain may comprise E490K, 1538K, and/or H537R mutations.

### V. Transcription Activator-Like Effector Nuclease Systems.

The programmable targeting nuclease may also be a transcription activator-like effector nuclease (TALEN) or the like. TALENs comprise a DNA-binding domain composed of highly conserved repeats derived from transcription activator-like effectors (TALEs) that are linked to a nuclease domain. TALEs are proteins secreted by plant pathogen Xanthomonas to alter transcription of genes in host plant cells. TALE repeat arrays may be engineered via modular protein design to target any DNA sequence of interest. Other transcription activator-like effector nuclease systems may comprise, but are not limited to, the repetitive sequence, transcription activator like effector (RipTAL) system from the bacterial plant pathogenic Ralstonia solanacearum species complex (Rssc). The nuclease domain of TALEs may be any nuclease domain as described above in Section (I) (c) (i).

vi. Meganucleases or Rare-Cutting Endonuclease Systems.

The programmable targeting nuclease may also be a meganuclease or derivative thereof. Meganucleases are endodeoxyribonucleases characterized by long recognition sequences, i.e., the recognition sequence generally ranges from about 12 base pairs to about 45 base pairs. As a consequence of this requirement, the recognition sequence generally occurs only once in any given genome. Among meganucleases, the family of homing endonucleases named LAGLIDADG has become a valuable tool for the study of genomes and genome engineering. Non-limiting examples of meganucleases that may be suitable for the instant disclosure include I-SceI, I-CreI, I-DmoI, or variants and combinations thereof. A meganuclease may be targeted to a specific nucleic acid sequence by modifying its recognition sequence using techniques well known to those skilled in the art.

The programmable targeting nuclease can be a rare-cutting endonuclease or derivative thereof. Rare-cutting endonucleases are site-specific endonucleases whose recognition sequence occurs rarely in a genome, such as only once in a genome. The rare-cutting endonuclease may recognize a 7-nucleotide sequence, an 8-nucleotide sequence, or longer recognition sequence. Non-limiting examples of rare-cutting endonucleases include NotI, AscI, PacI, AsiSI, SbfI, and FseI.

vii. Optional Additional Domains.

The programmable targeting nuclease may further comprise at least one nuclear localization signal (NLS), at least one cell-penetrating domain, at least one reporter domain, and/or at least one linker.

In general, an NLS comprises a stretch of basic amino acids. Nuclear localization signals are known in the art (see, e.g., Lange et al., J. Biol. Chem., 2007, 282:5101-5105). The NLS may be located at the N-terminus, the C-terminal, or in an internal location of the fusion protein.

A cell-penetrating domain may be a cell-penetrating peptide sequence derived from the HIV-1 TAT protein. The cell-penetrating domain may be located at the N-terminus, the C-terminal, or in an internal location of the fusion protein.

A programmable targeting nuclease may further comprise at least one linker. For example, the programmable targeting nuclease, the nuclease domain of the targeting nuclease, and other optional domains may be linked via one or more linkers. The linker may be flexible (e.g., comprising small, non-polar (e.g., Gly) or polar (e.g., Ser, Thr) amino acids). Examples of suitable linkers are well known in the art, and programs to design linkers are readily available (Crasto et al., Protein Eng., 2000, 13 (5): 3096-312). In alternate aspects, the programmable targeting nuclease, the cell cycle regulated protein, and other optional domains may be linked directly.

A programmable targeting nuclease may further comprise an organelle localization or targeting signal that directs a molecule to a specific organelle. A signal may be polynucleotide or polypeptide signal, or may be an organic or inorganic compound sufficient to direct an attached molecule to a desired organelle. Organelle localization signals can be as described in U.S. Patent Publication No. 20070196334, the disclosure of which is incorporated herein in its entirety.

### (d) Engineered Proteins

An engineered protein of the instant disclosure comprises one or more methylation polypeptides and one or more targeting polypeptides comprising a targeting domain which specifically binds one or more target methylation loci in one or more nucleic acid sequences encoding a susceptibility gene. In some aspects, components of the system are transiently expressed in a plant or plant cell.

Using the engineered proteins of the instant disclosure, the level of methylation of methylation sites at a target methylation locus can be modulated. The level of methylation can be modulated by varying the number of copies of a methylation polypeptide targeted to a locus. Targeting more than one copy of a methylation polypeptide can methylate methylation sites at a locus to a higher level than targeting a single copy of the methylation polypeptide. Multiple copies of a methylation polypeptide can be targeted to a single methylation locus using multiple targeting polypeptides, each comprising a targeting domain which specifically binds one or more target methylation loci in one or more nucleic acid sequences encoding a susceptibility gene. In some aspects, the targeting polypeptide comprises one or more domains encoding a CRISPR targeting system. When the targeting polypeptide comprises one or more domains encoding a CRISPR targeting system, multiple copies of a methylation polypeptide can be targeted to a single locus by engineering multiple CRISPR systems, each comprising a gRNA engineered to target a copy of the methylation polypeptide to different nucleic acid sequences within or adjacent to the target methylation locus. Thus, the level of methylation of one or more loci can be fine-tuned by varying the number and placement of gRNAs, to fine-tune expression of a susceptibility gene. For example, gene expression of a susceptibility gene critical for normal plant growth and development can be fine-tuned to provide disease resistance or tolerance while maintaining a certain level of expression needed for normal plant development.

Alternatively, multiple copies of a methylation polypeptide can be targeted to a locus using a targeting polypeptide engineered to target multiple copies of the methylation polypeptide to a target methylation locus. For instance, a SunTag targeting system described in the section below can target 40 or more copies of a methylation polypeptide to the target methylation locus. A combination of these approaches is also envisioned.

The level of methylation can also be modulated by targeting a combination of more than one methylation polypeptide to a target locus. As explained herein above in Section I(b), it was discovered that combinations of methylation polypeptides, when co-targeted to a locus, can create synergistic methylation of a target locus in a susceptibility gene to high levels to thereby provide robust and tunable modulation of gene expression. A combination of more than one methylation polypeptide can be targeted using multiple targeting polypeptides, each engineered to target one of the combination of proteins to the target methylation loci. A combination of more than one methylation polypeptide can also be targeted using one or more targeting polypeptides engineered to target a combination of more than one methylation polypeptide to methylation loci. Multiple targeting polypeptides and a targeting polypeptide engineered to target a combination of more than one methylation polypeptide can be as described in the section above. A combination of these approaches is also envisioned. In some aspects, the targeting polypeptide comprises one or more domains encoding one or more CRISPR targeting systems, each comprising a gRNA engineered to target more than one of a combination of methylation polypeptides to different nucleic acid sequences within or adjacent to the target methylation locus. In other aspects, the targeting polypeptide comprises one or more zinc finger DNA binding domains engineered to target more than one of a combination of methylation polypeptides to different nucleic acid sequences within or adjacent to the target methylation locus. In other aspects, the targeting polypeptide comprises one or more TALE proteins engineered to target more than one of a combination of methylation polypeptides to different nucleic acid sequences within or adjacent to the target methylation locus.

A combination of the systems described in this section can also be used to modulate expression of more than one susceptibility gene in a plant with great precision. By fine-tuning the expression of more than one susceptibility gene in a plant, optimal disease resistance with minimal pleiotropic negative effects can be achieved.

In some aspects, the targeting polypeptide is fused to the methylation polypeptide. In other aspects, the targeting polypeptide comprises an epitope and the methylation polypeptide comprises an affinity polypeptide that specifically binds to the epitope, and wherein binding of the affinity polypeptide to the epitope links the targeting polypeptide to the methylation polypeptide. In one aspect, the epitope is multimerized.

In some aspects, the targeting polypeptide comprises a zinc finger DNA binding domain. In other aspects, the targeting polypeptide comprises a TALE protein.

In some aspects, a targeting polypeptide comprises domains encoding one or more CRISPR targeting systems comprising one or more gRNA and an engineered polypeptide comprising a nuclease-deficient CAS9 polypeptide such as dCAS9, dCpf1 or dCjCas9, fused to one or more epitopes, and a methylation polypeptide is one or more methylation polypeptides wherein each methylation polypeptide comprises a methylation polypeptide and an affinity polypeptide that specifically binds to one or more epitopes of the targeting system to thereby target the one or more methylation polypeptides to the one or more target methylation loci. In some aspects, the targeting system is a CRISPR targeting system comprising a nuclease-deficient CAS9 polypeptide that is recombinantly fused to a multimerized epitope and a gRNA engineered to target more than one or more than one copy of a methylation polypeptide to a target locus in a plant susceptibility gene. For instance, the CRISPR targeting system can comprise about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 50, 55, 60, 65, 70, 75, 80, 85, 90, or 95 multimerized epitopes or more. A CRISPR targeting system can also comprise about 2-5, 2-10, 5-10, 7-15, 10-15, 10-20, 15-20, 20-25, 20-30, 30-35, 30-40, 35-40, 40-45, 40-50, 45-50, 50-55, 50-60, 55-60, 60-65, 60-70, 65-70, 70-75, 70-80, 75-80, 80-85, 80-90, 85-90, 90-95, 90-100, 95-100, or more than 100 multimerized epitopes.

In some aspects, all the epitopes are recognized by one antibody or antibody fragment. In such aspects, the system can target multiple copies of a methylation polypeptide comprising an antibody fragment that specifically binds the epitope of the targeting system. In some aspects, each of the epitopes is recognized by a different antibody or antibody fragment, or the multimerized epitopes comprise more than one group of epitopes, wherein each group of epitopes is recognized by a different antibody or antibody fragment. In such aspects, the system can target a combination of more than one methylation polypeptide wherein each of the combination of proteins comprises an antibody or antibody fragment that specifically binds to one or group of one epitope of the targeting system. In some aspects, the CRISPR targeting system is a SunTag targeting system and can be as described in International Patent Publication No. WO2016011070, the entire disclosure of which is incorporated herein in its entirety.

A. Resistance to Pathogens that Cause Cassava Bacterial Blight (CBB)

In some aspects, the engineered DNA methylation system of the instant disclosure is engineered to modulate the expression of one or more cassava susceptibility genes that cause CBB (CBB susceptibility gene). An engineered DNA methylation system engineered to modulate the expression of one or more CBB susceptibility genes comprises one or more methylation polypeptides and one or more targeting polypeptides, wherein the targeting polypeptides are engineered to target the methylation polypeptides to one or more target methylation loci in one or more CBB susceptibility genes to thereby mediate methylation of the one or more target methylation loci in the CBB susceptibility genes, and to thereby modify the expression of the one or more CBB susceptibility genes. In some aspects, a CBB susceptibility gene is a disease resistance gene, and the system is engineered to increase the expression of the resistance gene. In other aspects, a CBB susceptibility gene is an S gene, and the system is engineered to reduce the expression of the S gene.

CBB is caused by Xanthomonas axonopodis pv. manihotis that produces TALE proteins that bind TALE binding sites in promoter sequences of a number of S genes in cassava and other plants and activate the expression of the S genes to aid bacterial infection. Some TALE proteins specifically bind a single nucleic acid sequence. Other TALE proteins can bind a number of TALE binding sites having homologous but not necessarily identical nucleic acid sequences. Accordingly, in some aspects, the engineered DNA methylation system of the instant disclosure is engineered to modulate the expression of one or more CBB S genes comprising TALE binding sites in the promoter by methylating the TALE effector binding sites in the promoters of the genes.

In some aspects, the engineered DNA methylation system of the instant disclosure is engineered to modulate the expression of one or more CBB S genes comprising a TALE20 binding site in the promoter by methylating the TALE20 effector binding sites in the promoters of the genes. Non-limiting examples of cassava CBB S genes comprising TALE20 binding sites include the cassava MeSWEET10a gene, the cassava4.1_007568 pectate lyase gene, and the cassava4.1_007516 pectate lyase gene, among others. The 20 base pair TALE20 binding site in the MeSWEET10a promoter (ATAAACGCTTCTCGCCCATC; SEQ ID NO: 1) contains nine cytosines, including two in a CG sequence context. Methylation of all these cytosines can completely block TALE20 binding and gene activation by CBB, whereas methylation of less than all the cytosines can partially reduce the expression of the MeSWEET10a gene. The MeSWEET10a gene is essential for the growth and development of cassava. Accordingly, the engineered DNA methylation system can be engineered to fine-tune the expression of the MeSWEET10a gene by completely or partially methylating the TALE20 protein binding site in the promoter to provide precise control of the level of expression, thereby allowing for fine-tuning of the tradeoffs between pathogen resistance and normal plant growth and development. Additionally, expression of the MeSWEET10a gene is not essential for plant growth and development in leaves. Accordingly, the engineered DNA methylation system can also be engineered to specifically target methylation of the MeSWEET10a gene in leaves by specifically expressing the system in leaves using a leaf-specific promoter, also allowing for fine-tuning pathogen resistance and normal plant growth and development. Tissue-specific promoters can be as described in Section II below.

In some aspects, the engineered DNA methylation system modulates the expression of the MeSWEET10a gene by methylating the TALE20 protein binding site in the promoter. In some aspects, the engineered DNA methylation system modulates the expression of the cassava4.1_007568 pectate lyase gene by methylating the TALE20 protein binding site in the promoter. In some aspects, the engineered DNA methylation system modulates the expression of the cassava4.1_007516 pectate lyase gene by methylating the TALE20 protein binding site in the promoter.

In some aspects, the engineered DNA methylation system modulates the expression of more than one CBB S gene comprising a TALE protein binding site, by engineering one or more methylation systems to methylate the TALE protein binding site in the promoter of each gene. In some aspects, the engineered DNA methylation system modulates the expression of the MeSWEET10a gene, the cassava4.1_007516 pectate lyase gene, the cassava4.1_007568 pectate lyase gene, and any combination thereof by methylating the TALE20 protein binding site in the promoter of each gene. In some aspects, the engineered DNA methylation system modulates the expression of the MeSWEET10a gene and at least one more CBB S gene comprising a TALE20 protein binding site.

In some aspects, the engineered DNA methylation system comprises one or more CRISPR targeting systems. In one aspect, the CRISPR targeting system is a SunTag targeting system. In some aspects, the SunTag targeting system is engineered to target one or more copies of one or more methylation polypeptides to one or more nucleic acid sequences within or adjacent to one or more target methylation loci as described in Section I(a) to Section I(c). In some aspects, the one or more methylation polypeptides each comprises a methylation domain, wherein each methylation domain comprises SUVH2, SUVH9, DMS3, DRM2, DRM3, NRPE1 (largest subunit of Pol V), NRPD1 (largest subunit of Pol IV), CLSY1, NRPD2, RDR2, DCL3, AGO4, DRD1, RDM1, DMS4, KTF1, IDN2, SUVR2, or combinations thereof. In some aspects, the methylation domain comprises DMS3. In some aspects, the methylation domain comprises DRM2. In some aspects, the methylation domain comprises MQ1. In some aspects, the methylation domain comprises NRPD1. In some aspects, the methylation domain comprises DRM3 and NRPD1.

### B. Resistance to Cassava Brown Streak Disease (CBSD)

In some aspects, the engineered DNA methylation system of the instant disclosure is engineered to modulate the expression of one or more CBSD susceptibility genes. An engineered DNA methylation system engineered to modulate the expression of one or more CBSD susceptibility genes comprises one or more methylation polypeptides and one or more targeting polypeptides, wherein the targeting polypeptides are engineered to target the methylation polypeptides to one or more target methylation loci in one or more CBSD susceptibility genes to thereby mediate methylation of the one or more target methylation loci in the CBSD susceptibility genes, and to thereby modify the expression of the one or more CBSD susceptibility genes. Engineered DNA methylation systems that reduce the expression of the one or more genes can be as described above in Section I(a) to Section I(d). In some aspects, a CBSD susceptibility gene is a disease resistance gene, and the system is engineered to increase the expression of the resistance gene. In other aspects, a CBSD susceptibility gene is a susceptibility gene, and the system is engineered to reduce the expression of the resistance gene. In some aspects, a CBSD susceptibility gene is an S gene.

In some aspects, the engineered DNA methylation system is engineered to modulate the expression of the nCBP-1 and nCBP-2 eiIF4E genes, the SUVR2 genes, and combinations thereof. In some aspects, the engineered DNA methylation system is engineered to modulate the expression of an eif4e gene. In some aspects, the engineered DNA methylation system is engineered to modulate the expression of the nCBP-1 gene. In some aspects, the engineered DNA methylation system is engineered to modulate the expression of the nCBP-2 gene. In some aspects, the methylation domain comprises DMS3. In some aspects, the methylation domain comprises DRM2. In some aspects, the methylation domain comprises MQ1. In some aspects, the methylation domain comprises NRPD1. In some aspects, the methylation domain comprises DRM3 and NRPD1.

In some aspects, the engineered DNA methylation system comprises one or more CRISPR targeting systems. In one aspect, the CRISPR targeting system is a SunTag targeting system. In some aspects, the SunTag targeting system is engineered to target one or more copies of one or more methylation polypeptides to one or more nucleic acid sequences within or adjacent to one or more target methylation loci using methods described above in Section I(a) to Section I(c). In some aspects, the one or more methylation polypeptides comprise methylation domains comprising SUVH2, SUVH9, DMS3, DRM2, DRM3, NRPE1 (largest subunit of Pol V), NRPD1 (largest subunit of Pol IV), CLSY1, NRPD2, RDR2, DCL3, AGO4, DRD1, RDM1, DMS4, KTF1, IDN2, SUVR2, or combinations thereof. In some aspects, the methylation domain comprises DMS3. In some aspects, the methylation domain comprises NRPD1. In some aspects, the methylation domain comprises DRM3 and NRPD1.

### C. Aspects of Engineered Proteins

In some aspects, the targeting polypeptide of the engineered protein of the instant disclosure is a programmable targeting protein comprising a programmable, sequence-specific DNA-binding domain DNA binding domain of a programmable targeting system engineered to target one or more target methylation loci in one or more plant susceptibility genes. In some aspects, the targeting system comprises a targeting polypeptide comprising a targeting domain comprising a nuclease-deficient CAS9 protein (dCAS9) and optionally an epitope and one or more guide RNA. The engineered protein also comprises a methylation polypeptide comprising a methylation domain comprising a DRM2 protein fused to the targeting polypeptide or fused to an affinity polypeptide that specifically binds to the epitope. The targeting system targets the polypeptide to the target methylation loci to thereby mediate methylation of one or more methylation sites at the target methylation loci, and to thereby modulate the expression of the one or more plant susceptibility genes.

In one aspect, the plant is cassava, the susceptibility gene is MeSWEET10a, and the pathogen is a bacterial pathogen that causes cassava bacterial blight (CBB). In another aspect, the plant is cassava, the susceptibility gene is nCBP-1 and nCBP-2, and the pathogen is CBSV. In one aspect, the DRM2 protein is encoded by a nucleic acid sequence having about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 7.

In other aspects, the targeting polypeptide of the engineered protein of the instant disclosure is a programmable targeting protein comprising a programmable, sequence-specific DNA-binding domain DNA binding domain of a programmable targeting system engineered to target one or more target methylation loci in one or more plant susceptibility genes. In some aspects, the targeting system comprises a targeting polypeptide comprising a targeting domain comprising a nuclease-deficient CAS9 protein (dCAS9) and optionally an epitope and one or more guide RNA. In some aspects, the engineered protein also comprises a polypeptide comprising a methylation domain comprising a DMS3 protein, wherein the methylation polypeptide is linked to the targeting polypeptide. The methylation polypeptide can be fused to the targeting polypeptide or fused to an affinity polypeptide that specifically binds to the epitope. The targeting system targets the methylation polypeptide to the target methylation loci to thereby mediate methylation of one or more methylation sites at the target methylation loci, and to thereby modulate the expression of the one or more plant susceptibility genes.

In one aspect, the plant is cassava, the susceptibility gene is MeSWEET10a, and the pathogen is a bacterial pathogen that causes cassava bacterial blight (CBB). In another aspect, the plant is cassava, the susceptibility gene is nCBP-1 and nCBP-2, and the pathogen is CBSV.

In yet other aspects, the targeting polypeptide of the engineered protein of the instant disclosure is a programmable targeting protein comprising a programmable, sequence-specific DNA-binding domain DNA binding domain of a programmable targeting system engineered to target one or more target methylation loci in one or more plant susceptibility genes. In some aspects, the targeting system comprises a targeting polypeptide comprising a targeting domain comprising a nuclease-deficient CAS9 protein (dCAS9) and optionally an epitope and one or more guide RNA. In some aspects, the engineered protein also comprises a polypeptide comprising a methylation domain comprising a MQ1 protein, wherein the methylation polypeptide is linked to the targeting polypeptide. The methylation polypeptide can be fused to the targeting polypeptide or fused to an affinity polypeptide that specifically binds to the epitope. The targeting system targets the methylation polypeptide to the target methylation loci to thereby mediate methylation of one or more methylation sites at the target methylation loci, and to thereby modulate the expression of the one or more plant susceptibility genes.

In one aspect, the plant is cassava, the susceptibility gene is MeSWEET10a, and the pathogen is a bacterial pathogen that causes cassava bacterial blight (CBB). In another aspect, the plant is cassava, the susceptibility gene is nCBP-1 and nCBP-2, and the pathogen is CBSV. In one aspect, the methylation polypeptide is encoded by a nucleic acid sequence having about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 6.

In additional aspects, the engineered protein comprises a targeting polypeptide comprising a targeting domain comprising a zinc finger DNA binding domain which specifically binds to one or more target methylation loci in one or more plant susceptibility genes. The targeting polypeptide optionally comprises an epitope. In some aspects, the engineered DNA methylation system also comprises a methylation polypeptide comprising a methylation domain comprising a DRM2 protein. The methylation polypeptide can be fused to the targeting polypeptide or fused to an affinity polypeptide that specifically binds to the epitope of the methylation polypeptide. The targeting polypeptide targets the methylation to the target methylation loci to thereby mediate methylation of one or more methylation sites at the target methylation loci, and to thereby modulate the expression of the one or more plant susceptibility genes.

In one aspect, the plant is cassava, the susceptibility gene is MeSWEET10a, and the pathogen is a bacterial pathogen that causes cassava bacterial blight (CBB). In another aspect, the plant is cassava, the susceptibility gene is nCBP-1 and nCBP-2, and the pathogen is CBSV. In one aspect, the zinc finger DNA binding domain is encoded by a nucleic acid sequence having about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 5. In one aspect, the DRM2 protein is encoded by a nucleic acid sequence having about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 7.

In additional aspects, the engineered protein comprises a targeting polypeptide comprising a targeting domain comprising a zinc finger DNA binding domain which specifically binds to one or more target methylation loci in one or more plant susceptibility genes. The targeting polypeptide optionally comprises an epitope. In some aspects, the engineered DNA methylation system also comprises a methylation polypeptide comprising a methylation domain comprising a DMS3 protein. The methylation polypeptide can be fused to the targeting polypeptide or fused to an affinity polypeptide that specifically binds to the epitope of the methylation polypeptide. The targeting polypeptide targets the methylation to the target methylation loci to thereby mediate methylation of one or more methylation sites at the target methylation loci, and to thereby modulate the expression of the one or more plant susceptibility genes.

In one aspect, the plant is cassava, the susceptibility gene is MeSWEET10a, and the pathogen is a bacterial pathogen that causes cassava bacterial blight (CBB). In another aspect, the plant is cassava, the susceptibility gene is nCBP-1 and nCBP-2, and the pathogen is CBSV. In some aspects, the methylation polypeptide is fused to the targeting polypeptide. In one aspect, the zinc finger DNA binding domain is encoded by a nucleic acid sequence having about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 5.

In additional aspects, the engineered protein comprises a targeting polypeptide comprising a targeting domain comprising a zinc finger DNA binding domain which specifically binds to one or more target methylation loci in one or more plant susceptibility genes. The targeting polypeptide optionally comprises an epitope. In some aspects, the engineered DNA methylation system also comprises a methylation polypeptide comprising a methylation domain comprising a MQ1 protein. The methylation polypeptide can be fused to the targeting polypeptide or fused to an affinity polypeptide that specifically binds to the epitope of the methylation polypeptide. The targeting polypeptide targets the methylation to the target methylation loci to thereby mediate methylation of one or more methylation sites at the target methylation loci, and to thereby modulate the expression of the one or more plant susceptibility genes.

In one aspect, the plant is cassava, the susceptibility gene is MeSWEET10a, and the pathogen is a bacterial pathogen that causes cassava bacterial blight (CBB). In another aspect, the plant is cassava, the susceptibility gene is nCBP-1 and nCBP-2, and the pathogen is CBSV. In one aspect, the MQ1 protein is encoded by a nucleic acid sequence having about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 6. In one aspect, the zinc finger DNA binding domain is encoded by a nucleic acid sequence having about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 5.

In further aspects, the engineered protein comprises a targeting polypeptide comprising a targeting domain comprising a TALE protein which specifically binds to one or more target methylation loci in one or more plant susceptibility genes. The targeting polypeptide optionally comprises an epitope. The engineered DNA methylation system also comprises a methylation domain comprising a DRM2 protein. The methylation polypeptide is fused to the targeting polypeptide or fused to an affinity polypeptide that specifically binds to the epitope of the targeting polypeptide. The targeting polypeptide targets the methylation polypeptide to the target methylation loci to thereby mediate methylation of one or more methylation sites at the target methylation loci, and to thereby modulate the expression of the one or more plant susceptibility genes.

In one aspect, the plant is cassava, the susceptibility gene is MeSWEET10a, and the pathogen is a bacterial pathogen that causes cassava bacterial blight (CBB). In another aspect, the plant is cassava, the susceptibility gene is nCBP-1 and nCBP-2, and the pathogen is CBSV. In one aspect, the DRM2 protein is encoded by a nucleic acid sequence having about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 7.

In further aspects, the engineered protein comprises a targeting polypeptide comprising a targeting domain comprising a TALE protein which specifically binds to one or more target methylation loci in one or more plant susceptibility genes. The targeting polypeptide optionally comprises an epitope. The engineered DNA methylation system also comprises a methylation domain comprising a DMS3 protein. The methylation polypeptide is fused to the targeting polypeptide or fused to an affinity polypeptide that specifically binds to the epitope of the targeting polypeptide. The targeting polypeptide targets the methylation polypeptide to the target methylation loci to thereby mediate methylation of one or more methylation sites at the target methylation loci, and to thereby modulate the expression of the one or more plant susceptibility genes.

In one aspect, the plant is cassava, the susceptibility gene is MeSWEET10a, and the pathogen is a bacterial pathogen that causes cassava bacterial blight (CBB). In another aspect, the plant is cassava, the susceptibility gene is nCBP-1 and nCBP-2, and the pathogen is CBSV.

In further aspects, the engineered protein comprises a targeting polypeptide comprising a targeting domain comprising a TALE protein which specifically binds to one or more target methylation loci in one or more plant susceptibility genes. The targeting polypeptide optionally comprises an epitope. The engineered DNA methylation system also comprises a methylation domain comprising a MQ1 protein. The methylation polypeptide is fused to the targeting polypeptide or fused to an affinity polypeptide that specifically binds to the epitope of the targeting polypeptide. The targeting polypeptide targets the methylation polypeptide to the target methylation loci to thereby mediate methylation of one or more methylation sites at the target methylation loci, and to thereby modulate the expression of the one or more plant susceptibility genes.

In one aspect, the plant is cassava, the susceptibility gene is

MeSWEET10a, and the pathogen is a bacterial pathogen that causes cassava bacterial blight (CBB). In another aspect, the plant is cassava, the susceptibility gene is nCBP-1 and nCBP-2, and the pathogen is CBSV. In one aspect, the MQ1 protein is encoded by a nucleic acid sequence having about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 6.

In some aspects, the engineered protein comprises a methylation polypeptide comprising a DNA methylation domain of a DMS3 protein fused to a zinc finger DNA binding domain programmed to target the engineered protein to a locus in a promoter region of a cassava MeSWEET10a gene. In some aspects, the DMS3 protein is encoded by a nucleic acid sequence comprising at least about 75% or more, at least about 85% or more, at least about 95% or more, or 100% sequence identity with a nucleic acid sequence of SEQ ID NO: 2 and wherein the programmable targeting protein comprises an amino acid sequence comprising at least about 75% or more, at least about 85% or more, at least about 95% or more, or 100% sequence identity with the amino acid sequence of SEQ ID NO: 5.

In some aspects, the engineered protein comprises a methylation polypeptide comprising a DNA methylation domain of a MQ1 protein fused to a nuclease-deficient CAS9 protein (dCAS9) of a CRISPR/Cas nuclease system comprising a gRNA comprising a sequence which binds to a nucleotide sequence in the target nucleic acid sequence to thereby target the engineered protein to a locus in a promoter region of a cassava MeSWEET10a gene. In some aspects, the MQ1 protein is encoded by a nucleic acid sequence comprising at least about 75% or more, at least about 85% or more, at least about 95% or more, or 100% sequence identity with a nucleic acid sequence of SEQ ID NO: 6 and wherein the gRNA is selected from a gRNA selected from a gRNA comprising SEQ ID NO: 3, a gRNA comprising SEQ ID NO: 4, or a combination thereof.

In some aspects, the engineered protein comprises a DRM2 methylation polypeptide comprising an affinity polypeptide and a dCAS9 protein of a CRISPR/Cas nuclease system comprising a gRNA comprising a sequence which binds to a nucleotide sequence in the target nucleic acid sequence to thereby target the engineered protein to a locus in a promoter region of a cassava MeSWEET10a gene, wherein the dCas9 protein comprises an epitope that specifically binds to the affinity polypeptide. In some aspects, the gRNA is selected from a gRNA selected from a gRNA comprising the nucleic acid sequence of SEQ ID NO: 3, a gRNA comprising the nucleic acid sequence of SEQ ID NO: 4, or a combination thereof.

In some aspects, the engineered protein comprises a DRM2 methylation polypeptide comprising an affinity polypeptide and a dCAS9 protein of a CRISPR/Cas nuclease system comprising a gRNA comprising a sequence which binds to a nucleotide sequence in the target nucleic acid sequence to thereby target the engineered protein to a locus in a promoter region of a cassava nCBP1 gene, wherein the dCas9 protein comprises a multimerized epitope that specifically binds to the affinity polypeptide. In some aspects, the gRNA is selected from a gRNA selected from a gRNA comprising the nucleic acid sequence of SEQ ID NO: 8, a gRNA comprising the nucleic acid sequence of SEQ ID NO: 9, or a combination thereof.

In some aspects, the engineered protein comprises a DRM2 methylation polypeptide comprising an affinity polypeptide and a dCAS9 protein of a CRISPR/Cas nuclease system comprising a gRNA comprising a sequence which binds to a nucleotide sequence in the target nucleic acid sequence to thereby target the engineered protein to a locus in a promoter region of a cassava nCBP2 gene, wherein the dCas9 protein comprises a multimerized epitope that specifically binds to the affinity polypeptide. In some aspects, the gRNA is selected from a gRNA selected from a gRNA comprising the nucleic acid sequence of SEQ ID NO: 10, a gRNA comprising the nucleic acid sequence of SEQ ID NO: 11, or a combination thereof.

### II. Expression Constructs

A further aspect of the present disclosure provides expression constructs encoding the engineered proteins described herein above in Section I. In some aspects, the nucleic acid constructs encode the engineered protein described in Section I(d). The expression constructs comprise a promoter operably linked to a nucleic acid sequence encoding the engineered protein.

Any of the engineered proteins including multi-component engineered proteins described herein are to be considered modular, in that the different components may optionally be distributed among two or more nucleic acid constructs as described herein. The nucleic acid constructs may be DNA or RNA, linear or circular, single-stranded or double-stranded, or any combination thereof. The nucleic acid constructs may be codon-optimized for efficient translation into protein, and possibly for transcription into an RNA donor polynucleotide transcript in the cell of interest. Codon optimization programs are available as freeware or from commercial sources.

The nucleic acid constructs can be used to express one or more components of the system for later introduction into a cell to be genetically modified. Alternatively, the nucleic acid constructs can be introduced into the cell to genetically modify the cell or plant for expression of the engineered proteins in the cell. In some aspects, the nucleic acid constructs transiently express the various components of the system. Transiently expressing the system in a plant overcomes the cumbersome regulatory hurdles required for traditionally genetically modified crops.

Expression constructs generally comprise DNA coding sequences operably linked to at least one promoter control sequence for expression in a cell of interest. Promoter control sequences may control expression of the transposase, the programmable targeting nuclease, the donor polynucleotide, or combinations thereof in bacterial (e.g., E. coli) cells or eukaryotic (e.g., yeast, insect, mammalian, or plant) cells. Suitable bacterial promoters include, without limit, T7 promoters, lac operon promoters, trp promoters, tac promoters (which are hybrids of trp and lac promoters), variations of any of the foregoing, and combinations of any of the foregoing. Non-limiting examples of suitable eukaryotic promoters include constitutive, regulated, or cell- or tissue-specific promoters. As explained above, methylation of the MeSWEET10a gene can be targeted in leaves by specifically expressing the engineered proteins of the instant disclosure in leaves using a leaf-specific promoter, allowing for fine-tuning pathogen resistance and normal plant growth and development.

Suitable eukaryotic constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or combinations of any of the foregoing. Examples of suitable eukaryotic regulated promoter control sequences include, without limit, those regulated by heat shock, metals, steroids, antibiotics, or alcohol. Non-limiting examples of tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIlb promoter, ICAM-2 promoter, INF-β promoter, Mb promoter, NphsI promoter, OG-2 promoter, SP-B promoter, SYN1 promoter, and WASP promoter.

Promoters may also be plant-specific promoters, or promoters that may be used in plants. A wide variety of plant promoters are known to those of ordinary skill in the art, as are other regulatory elements that may be used alone or in combination with promoters. Preferably, promoter control sequences control expression in cassava, such as promoters disclosed in Wilson et al., 2017, The New Phytologoist, 213 (4): 1632-1641, the disclosure of which is incorporated herein in its entirety.

Promoters may be divided into two types, namely, constitutive promoters and non-constitutive promoters. Constitutive promoters are classified as providing for a range of constitutive expression. Thus, some are weak constitutive promoters, and others are strong constitutive promoters. Non-constitutive promoters include tissue-preferred promoters, tissue-specific promoters, cell-type specific promoters, and inducible promoters. Suitable plant-specific constitutive promoter control sequences include, but are not limited to, a CaMV35S promoter, CaMV 19S, GOS2, Arabidopsis At6669 promoter, Rice cyclophilin, Maize H3 histone, Synthetic Super MAS, an opine promoter, a plant ubiquitin (Ubi) promoter, an actin 1 (Act-1) promoter, pEMU, Cestrum yellow leaf curling virus promoter (CYMLV promoter), and an alcohol dehydrogenase 1 (Adh-1) promoter. Other constitutive promoters include those in U.S. Pat. Nos. 5,659,026; 5,608,149; 5,608,144; 5,604,121; 5,569,597; 5,466,785; 5,399,680; 5,268,463; and 5,608,142.

Regulated plant promoters respond to various forms of environmental stresses, or other stimuli, including, for example, mechanical shock, heat, cold, flooding, drought, salt, anoxia, pathogens such as bacteria, fungi, and viruses, and nutritional deprivation, including deprivation during times of flowering and/or fruiting, and other forms of plant stress. For example, the promoter may be a promoter which is induced by one or more, but not limited to one of the following: abiotic stresses such as wounding, cold, desiccation, ultraviolet-B, heat shock or other heat stress, drought stress or water stress. The promoter may further be one induced by biotic stresses including pathogen stress, such as stress induced by a virus or fungi, stresses induced as part of the plant defense pathway or by other environmental signals, such as light, carbon dioxide, hormones or other signaling molecules such as auxin, hydrogen peroxide and salicylic acid, sugars and gibberellin or abscisic acid and ethylene. Suitable regulated plant promoter control sequences include, but are not limited to, salt-inducible promoters such as RD29A; drought-inducible promoters such as maize rab17 gene promoter, maize rab28 gene promoter, and maize Ivr2 gene promoter; heat-inducible promoters such as heat tomato hsp80-promoter from tomato.

Tissue-specific promoters may include, but are not limited to, fiber-specific, green tissue-specific, root-specific, stem-specific, flower-specific, callus-specific, pollen-specific, egg-specific, and seed coat-specific. Suitable tissue-specific plant promoter control sequences include, but are not limited to, leaf-specific promoters [such as described, for example, by Yamamoto et al., Plant J. 12:255-265, 1997; Kwon et al., Plant Physiol. 105:357-67, 1994; Yamamoto et al., Plant Cell Physiol. 35:773-778, 1994; Gotor et al., Plant J. 3:509-18, 1993; Orozco et al., Plant Mol. Biol. 23:1129-1138, 1993; and Matsuoka et al., Proc. Natl. Acad. Sci. USA 90:9586-9590, 1993], seed-preferred promoters [e.g., from seed-specific genes (Simon et al., Plant Mol. Biol. 5. 191, 1985; Scofield et al., J. Biol. Chem. 262:12202, 1987; Baszczynski et al., Plant Mol. Biol. 14:633, 1990), Brazil Nut albumin (Pearson et al., Plant Mol. Biol. 18:235-245, 1992), legumin (Ellis et al., Plant Mol. Biol. 10:203-214, 1988), Glutelin (rice) (Takaiwa et al., Mol. Gen. Genet. 208:15-22, 1986; Takaiwa et al., FEBS Letts. 221:43-47, 1987), Zein (Matzke et al., Plant Mol Biol, 143:323-32, 1990), napA (Stalberg et al., Planta 199:515-519, 1996), Wheat SPA (Albanietal, Plant Cell, 9:171-184, 1997), sunflower oleosin (Cummins et al., Plant Mol. Biol. 19:873-876, 1992)], endosperm specific promoters [e.g., wheat LMW and HMW, glutenin-1 (Mol Gen Genet 216:81-90, 1989; NAR 17:461-2), wheat a, b, and g gliadins (EMBO3: 1409-15, 1984), Barley Itrl promoter, barley B1, C, D hordein (Theor Appl Gen 98:1253-62, 1999; Plant J 4:343-55, 1993; Mol Gen Genet 250:750-60, 1996), Barley DOF (Mena et al., The Plant Journal, 116 (1): 53-62, 1998), Biz2 (EP99106056.7), Synthetic promoter (Vicente-Carbajosa et al., Plant J. 13:629-640, 1998), rice prolamin NRP33, rice-globulin Glb-1 (Wu et al., Plant Cell Physiology 39 (8) 885-889, 1998), rice alpha-globulin REB/OHP-1 (Nakase et al., Plant Mol. Biol. 33:513-S22, 1997), rice ADP-glucose PP (Trans Res 6:157-68, 1997), maize ESR gene family (Plant J 12:235-46, 1997), sorgum gamma-kafirin (PMB 32:1029-35, 1996)], embryo-specific promoters [e.g., rice OSH1 (Sato et al., Proc. Natl. Acad. Sci. USA, 93:8117-8122), KNOX (Postma-Haarsma et al., Plant Mol. Biol. 39:257-71, 1999), rice oleosin (Wu et al., J. Biochem., 123:386, 1998)], and flower-specific promoters [e.g., AtPRP4, chalene synthase (chsA) (Van der Meer et al., Plant Mol. Biol. 15, 95-109, 1990), LAT52 (Twell et al., Mol. Gen Genet. 217:240-245; 1989), apetala-3].

Any of the promoter sequences may be wild type or may be modified for more efficient or efficacious expression. The DNA coding sequence also may be linked to a polyadenylation signal (e.g., SV40 polyA signal, bovine growth hormone (BGH) polyA signal, etc.) and/or at least one transcriptional termination sequence. In some situations, the complex or fusion protein may be purified from the bacterial or eukaryotic cells.

Nucleic acids encoding one or more components of an engineered protein can be present in a construct. Suitable constructs include plasmid constructs, viral constructs, and self-replicating RNA (Yoshioka et al., Cell Stem Cell, 2013, 13:246-254). For instance, the nucleic acid encoding one or more components of an engineered DNA methylation system and/or transcription activation system may be present in a plasmid construct.

Non-limiting examples of suitable plasmid constructs include pUC, pBR322, pET, pBluescript, and variants thereof. Alternatively, the nucleic acid encoding one or more components of an engineered DNA methylation system and/or transcription activation system may be part of a viral vector (e.g., lentiviral vectors, adeno-associated viral vectors, adenoviral vectors, and so forth).

The plasmid or viral vector may comprise additional expression control sequences (e.g., enhancer sequences, Kozak sequences, polyadenylation sequences, transcriptional termination sequences, etc.), selectable reporter sequences (e.g., antibiotic resistance genes), origins of replication, T-DNA border sequences, and the like. The plasmid or viral vector may further comprise RNA processing elements such as glycine tRNAs, or Csy4 recognition sites. Such RNA processing elements can, for instance, intersperse polynucleotide sequences encoding multiple gRNAs under the control of a single promoter to produce the multiple gRNAs from a transcript encoding the multiple gRNAs. When a cys4 recognition cite is used, a vector may further comprise sequences for expression of Csy4 RNAse to process the gRNA transcript. Additional information about vectors and use thereof may be found in “Current Protocols in Molecular Biology”, Ausubel et al., John Wiley & Sons, New York, 2003, or “Molecular Cloning: A Laboratory Manual”, Sambrook & Russell, Cold Spring Harbor Press, Cold Spring Harbor, NY, 3rd edition, 2001.

Another aspect of the instant disclosure encompasses an expression construct for methylating a target nucleic acid sequence in a plant pathogen susceptibility gene. The expression construct comprises a promoter operably linked to a nucleic acid sequence encoding an engineered protein. The engineered protein comprises a programmable targeting polypeptide comprising a programmable sequence-specific DNA binding domain, wherein the programmable DNA binding domain binds a target DNA sequence in a target methylation locus in a polynucleotide encoding a plant pathogen susceptibility gene. The programmable targeting protein comprises a nuclease-deficient CAS9 protein (dCAS9) and optionally an epitope; and one or more guide RNA. The engineered protein also comprises a methylation polypeptide comprising a DNA methylation domain of a DRM2 protein, a DMS3 protein, or an MQ1 protein. The methylation polypeptide is fused to the targeting polypeptide or fused to an affinity polypeptide that specifically binds to the epitope.

Yet another aspect of the instant disclosure encompasses an expression construct for methylating a target nucleic acid sequence in a plant pathogen susceptibility gene. The expression construct comprises a promoter operably linked to a nucleic acid sequence encoding an engineered protein. The engineered protein comprises a programmable targeting polypeptide comprising a programmable sequence-specific DNA binding domain of a zinc finger DNA binding protein programmed to specifically bind one or more target DNA sequence in a target methylation locus in a polynucleotide encoding a plant pathogen susceptibility gene, wherein the targeting polypeptide optionally comprises an epitope. The engineered protein also comprises a methylation polypeptide comprising a DNA methylation domain of a DMS3 protein, a DRM2 protein, an MQ1 protein. The methylation polypeptide is fused to the targeting polypeptide or fused to an affinity polypeptide that specifically binds to the epitope.

An additional aspect of the instant disclosure encompasses an expression construct for methylating a target nucleic acid sequence in a plant pathogen susceptibility gene. The expression construct comprises a promoter operably linked to a nucleic acid sequence encoding an engineered protein, and the engineered protein comprises a programmable targeting polypeptide comprising a programmable sequence-specific DNA binding domain of a TALE DNA binding protein programmed to specifically bind one or more target DNA sequence in a target methylation locus in a polynucleotide encoding a plant pathogen susceptibility gene, wherein the targeting polypeptide optionally comprises an epitope. The engineered protein also comprises a methylation polypeptide comprising a DNA methylation domain of a DMS3 protein, a DRM2 protein, an MQ1 protein. The methylation polypeptide is fused to the targeting polypeptide or fused to an affinity polypeptide that specifically binds to the epitope.

One aspect of the instant disclosure encompasses one or more vectors comprising one or more expression constructs for methylating a target nucleic acid sequence in a plant pathogen susceptibility gene. The constructs comprise a promoter operably linked to a nucleic acid sequence encoding an engineered protein. The constructs and the engineered protein can be as described herein above.

### III. Plants

One aspect of the instant disclosure encompasses a plant cell, a plant part, or a plant comprising an engineered protein described in Section I above. One or more components of the engineered protein in the cell may be encoded by one or more nucleic acid constructs of a system of nucleic acid constructs as described in Section II above. Further, as explained in Section I(b) above, once RNA-directed DNA methylation is established at a location in the genome, it is often efficiently maintained and can be heritable in subsequent plant generations, even after removal of the original trigger that initially caused methylation. Accordingly, an aspect of the present disclosure comprises an epigenetically modified disease-resistant plant, plant part, or plant cell comprising one or more methylated target methylation loci in one or more plant susceptibility genes.

The cell may be a plant cell, a plant part, or a plant. Plant cells include germ cells and somatic cells. Non-limiting examples of plant cells include parenchyma cells, sclerenchyma cells, collenchyma cells, xylem cells, and phloem cells. Plant parts include, but are not limited to, stems, roots, ovules, stamens, leaves, embryos, meristematic regions, callus tissue, gametophytes, sporophytes, pollen, microspores, and the like. The plant can be a monocot plant or a dicot plant. For instance, the plant can be soybean; maize; sugar cane; beet; tobacco; wheat; barley; poppy; rape; sunflower; alfalfa; sorghum; rose; carnation; gerbera; carrot; tomato; lettuce; chicory; pepper; melon; cabbage; oat; rye; cotton; millet; flax; potato; pine; walnut; citrus (including oranges, grapefruit, etc.); hemp; oak; rice; petunia; orchids; Arabidopsis; broccoli; cauliflower; brussel sprouts; onion; garlic; leek; squash; pumpkin; celery; pea; bean (including various legumes); strawberries; grapes; apples; cherries; pears; peaches; banana; palm; cocoa; cucumber; pineapple; apricot; plum; sugar beet; lawn grasses; maple; teosinte; Tripsacum; Coix; triticale; safflower; peanut; cassava, and olive. In some aspects, the plant is a disease-resistant cassava plant. In some aspects, the plant is a CBB-resistant cassava plant, a CBSD-resistant cassava plant, or a cassava plant resistant to CBB and CBSD.

The disclosure also provides an agricultural product produced by any of the described transgenic plants, plant parts, and plant seeds. Agricultural products include, but are not limited to, plant extracts, proteins, amino acids, carbohydrates, fats, oils, polymers, vitamins, and the like.

One aspect of the instant disclosure encompasses a plant or plant cell comprising one or more expression constructs for methylating a target nucleic acid sequence in a plant pathogen susceptibility gene or one or more vectors comprising the one or more constructs. The constructs comprise a promoter operably linked to a nucleic acid sequence encoding an engineered protein. The constructs, the vectors, and the engineered protein can be as described herein above.

Another aspect of the instant disclosure encompasses a plant or plant cell comprising one or more methylated sites in a methylation locus in a plant pathogen susceptibility gene.

In some aspects, the plant is cassava. When the plant is cassava, the susceptibility gene can be MeSWEET10a. In some aspects, the plant is cassava, the susceptibility gene is MeSWEET10a, and the pathogen is a bacterial pathogen that causes cassava bacterial blight (CBB). The pathogen that causes CBB is can be a Xanthomonas sp. In some aspects, the plant is cassava, the plant pathogen susceptibility gene is MeSWEET10a, and the pathogen is a Xanthomonas sp.

When the plant is cassava, the plant pathogen susceptibility gene can also be nCBP-1, nCBP-2, or combinations thereof. In some aspects, the plant pathogen susceptibility gene is nCBP-1 and nCBP-2. In other aspects, the plant is cassava, the plant pathogen susceptibility gene is nCBP-1 and nCBP-2, and the pathogen is a viral pathogen that causes cassava brown streak disease. The viral pathogen that causes cassava brown streak disease can be selected from cassava brown streak virus (CBSV), Uganda CBSV, or a combination thereof. In some aspects, the plant is cassava, the susceptibility gene is nCBP-1 and nCBP-2, and the pathogen is CBSV.

Yet another aspect of the instant disclosure encompasses a disease-resistant cassava plant. The cassava plant comprises one or more methylated sites in a promoter region of a MeSWEET10a susceptibility gene. The cassava plant is resistant to a Xanthomonas sp. that causes cassava bacterial blight (CBB).

An additional aspect of the instant disclosure encompasses disease-resistant cassava plant. The cassava plant comprises one or more methylated sites in a promoter region of an nCBP-1 gene susceptibility and one or more methylated sites in a promoter region of an nCBP-2 susceptibility gene. The cassava plant is resistant to a viral pathogen that causes cassava brown streak disease. In some aspects, the viral pathogen that causes cassava brown streak disease is selected from cassava brown streak virus (CBSV), Uganda CBSV, or a combination thereof.

One aspect of the instant disclosure encompasses a disease-resistant cassava plant. The cassava plant comprises one or more methylated sites in a promoter region of an nCBP-1 gene susceptibility and one or more methylated sites in a promoter region of an nCBP-2 susceptibility gene. The cassava plant is resistant to CBSV.

### IV. Methods

A further aspect of the present disclosure provides a method of engineering disease resistance or tolerance in a plant. In a method of the instant disclosure, the cell can be ex vivo or in vivo. The method comprises methylating one or more target methylation loci in one or more plant susceptibility genes to thereby modify the expression of the one or more plant susceptibility genes, to thereby produce an engineered disease-resistant plant.

Methylating the one or more target methylation loci comprises introducing an engineered protein of the instant disclosure into a plant or plant cell, and growing the plant or plant cell under conditions whereby the one or more loci are methylated, thereby generating an engineered plant or plant cell comprising one or more methylated loci that improve disease resistance or tolerance of the plant cell. Optionally, the method further comprises removing the engineered DNA methylation system from the plant or plant cell to thereby generate a disease-resistant plant that does not contain transgenes or any change in the DNA sequence. The locus can be in a chromosomal DNA, organellar DNA, or extrachromosomal DNA. In some aspects, the method can generate a disease-resistant cassava plant. In some aspects, the plant is a CBB-resistant cassava plant, a CBSD-resistant cassava plant, or a cassava plant resistant to CBB and CBSD.

The engineered system can be as described in Section I; nucleic acid constructs encoding one or more components of the engineered system can be as described in Section II; and plant cells, plant parts, or plants can be as described in Section III.

Yet another aspect of the instant disclosure encompasses a method of generating a disease resistant or tolerant plant. The method comprises the steps of (a) introducing one or more expression constructs expressing an engineered protein or one or more vectors comprising the one or more expression constructs into a plant or plant cell; (b) cultivating the plant or plant cell under conditions sufficient for the engineered protein is targeted to the target methylation loci in the one or more plant pathogen susceptibility genes, thereby generating an engineered plant or plant cell comprising one or more methylated loci, thereby generating the disease resistant or tolerant plant; and (c) optionally removing the one or more expression or one or more one or more vectors from the plant or plant cell. The constructs, the vectors, and the engineered protein can be as described herein above.

In some aspects, the plant is cassava. When the plant is cassava, the susceptibility gene can be MeSWEET10a. In some aspects, the plant is cassava, the susceptibility gene is MeSWEET10a, and the pathogen is a bacterial pathogen that causes cassava bacterial blight (CBB). The pathogen that causes CBB is can be a Xanthomonas sp. In some aspects, the plant is cassava, the plant pathogen susceptibility gene is MeSWEET10a, and the pathogen is a Xanthomonas sp.

When the plant is cassava, the plant pathogen susceptibility gene can also be nCBP-1, nCBP-2, or combinations thereof. In some aspects, the plant pathogen susceptibility gene is nCBP-1 and nCBP-2. In other aspects, the plant is cassava, the plant pathogen susceptibility gene is nCBP-1 and nCBP-2, and the pathogen is a viral pathogen that causes cassava brown streak disease. The viral pathogen that causes cassava brown streak disease can be selected from cassava brown streak virus (CBSV), Uganda CBSV, or a combination thereof. In some aspects, the plant is cassava, the susceptibility gene is nCBP-1 and nCBP-2, and the pathogen is CBSV.

(a) Introduction into the Cell

The method comprises introducing the engineered DNA methylation system into a cell of interest. The engineered DNA methylation system may be introduced into the cell as a purified isolated composition, purified isolated components of a composition, as one or more nucleic acid constructs encoding the engineered system, or combinations thereof. Further, components of the engineered DNA methylation system can be separately introduced into a cell. For example, a transposase, a donor polynucleotide, and a programmable targeting nuclease can be introduced into a cell sequentially or simultaneously.

The engineered DNA methylation system described above may be introduced into the cell by a variety of means. Suitable delivery means include microinjection, electroporation, sonoporation, biolistics, calcium phosphate-mediated transfection, cationic transfection, liposomes and other lipids, dendrimer transfection, heat shock transfection, nucleofection transfection, gene gun delivery, dip transformation, supercharged proteins, cell-penetrating peptides, viral vectors, magnetofection, lipofection, impalefection, optical transfection, Agrobacterium tumefaciens mediated foreign gene transformation, proprietary agent-enhanced uptake of nucleic acids, and delivery via liposomes, immunoliposomes, virosomes, or artificial virions. The choice of means of introducing the system into a cell can and will vary depending on the cell, or the system or nucleic acid nucleic acid constructs encoding the system, among other variables.

### (b) Culturing a Plant

The method further comprises growing the plant, plant part, or plant cell under appropriate conditions such that the one or more target loci are methylated. When the cell is in tissue ex vivo, or in vivo within a plant or within a plant part, the plant part and/or plant may also be maintained under appropriate conditions for insertion of the donor polynucleotide. In general, the plant, plant part, or plant cell is maintained under conditions appropriate for cell growth and/or maintenance. Those of skill in the art appreciate that methods for culturing plant cells are known in the art and may and will vary depending on the cell type. Routine optimization may be used, in all cases, to determine the best techniques for a particular cell type. See, for example, Santiago et al. (2008) PNAS 105:5809-5814; Moehle et al. (2007) PNAS 104:3055-3060; Urnov et al. (2005) Nature 435:646-651; and Lombardo et al. (2007) Nat. Biotechnology 25:1298-1306; Taylor et al., (2012) Tropical Plant Biology 5:127-139.

### V. Kits

A further aspect of the present disclosure provides kits for generating an epigenetically modified plant, plant part, or plant cell. The kit comprises one or more engineered DNA methylation protein detailed above in Section I, one or more expression construct for expressing the engineered protein, or a vector comprising the expression constructs described above in Section II. Alternatively, the kit may comprise one or more plants, plant parts, plant cell culture, or plant cells comprising the one or more engineered proteins, the one or more expression constructs, the one or more vectors, or any combination thereof.

The kits may further comprise transfection reagents, cell growth media, selection media, in-vitro transcription reagents, nucleic acid purification reagents, protein purification reagents, buffers, and the like. The kits provided herein generally include instructions for carrying out the methods detailed above. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), an internet address that provides the instructions, and the like. As used herein, the term “instructions” may include the address of an internet site that provides the instructions.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

The terms ‘resistance’ and ‘tolerance’ are used interchangeably and refer to a plant having reduced pathogen growth on or in the plant or reduced impact of pathogen growth.

As used herein, the term “gene” refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

As used herein, the term “engineered” when applied to a targeting protein refers to targeting proteins modified to specifically recognize and bind to a nucleic acid sequence at or near a target methylation locus. A “genetically modified” plant refers to a cell in which the nuclear, organellar or extrachromosomal nucleic acid sequences of a cell have been modified, i.e., the cell contains at least one nucleic acid sequence that has been engineered to contain an insertion of at least one nucleotide, a deletion of at least one nucleotide, and/or a substitution of at least one nucleotide.

An “epigenetically modified” cell refers to a cell in which the nuclear, organellar or extrachromosomal nucleic acid sequences of a cell are not modified, but wherein the phenotype of the cell is modified.

The terms “genome modification” and “genome editing” refer to processes by which a specific nucleic acid sequence in a genome is changed such that the nucleic acid sequence is modified. The nucleic acid sequence may be modified to comprise an insertion of at least one nucleotide, a deletion of at least one nucleotide, and/or a substitution of at least one nucleotide. The modified nucleic acid sequence is inactivated such that no product is made. Alternatively, the nucleic acid sequence may be modified such that an altered product is made.

The term “heterologous” refers to an entity that is not native to the cell or species of interest.

The terms “nucleic acid” and “polynucleotide” refer to a deoxyribonucleotide or ribonucleotide polymer, in linear or circular conformation. For the purposes of the present disclosure, these terms are not to be construed as limiting with respect to the length of a polymer. The terms may encompass known analogs of natural nucleotides, as well as nucleotides that are modified in the base, sugar and/or phosphate moieties. In general, an analog of a particular nucleotide has the same base-pairing specificity, i.e., an analog of A will base-pair with T. The nucleotides of a nucleic acid or polynucleotide may be linked by phosphodiester, phosphothioate, phosphoramidite, phosphorodiamidate bonds, or combinations thereof.

The terms “polypeptide” and “protein” are used interchangeably to refer to a polymer of amino acid residues.

As used herein, the terms “target site”, “target sequence”, or “methylation locus” refer to a nucleic acid sequence comprising one or more methylation sites, wherein the target nucleic acid sequence defines a portion of a nucleic acid sequence comprising one or more methylation sites to be modified or edited and which a DNA methylation composition is engineered to target.

The terms “upstream” and “downstream” refer to locations in a nucleic acid sequence relative to a fixed position. Upstream refers to the region that is 5′ (i.e., near the 5′ end of the strand) to the position, and downstream refers to the region that is 3′ (i.e., near the 3′ end of the strand) to the position.

The terms “specific binding” or “specifically binds”, when used in the context of polypeptides or proteins, relates to binding by specific amino acid sequence(s) in a polypeptide comprising a “nucleic acid binding domain,” which recognizes and specifically binds a nucleic acid (e.g., DNA) target sequence of interest. As used herein, the term “specifically binds” refers to that binding affinity of the nucleic acid binding domain of a polypeptide as described herein, to a target DNA sequence of interest, which is measurably higher than the binding affinity of the same polypeptide to a generally comparable, but non-target DNA sequence. The binding affinity of the nucleic acid binding domain of a polypeptide to a nucleic acid sequence can be determined using any of many means known to those of ordinary skill in the art. A nucleic acid binding domain of a polypeptide that “specifically binds” to a target nucleic acid sequence, detectably binds the target nucleic acid sequence of interest by a factor of at least 1.5-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, or at least 20-fold, or more relative to the same polypeptide binding to non-target nucleic acid sequences, including to the substantial exclusion of non-target DNA sequences. The Kd of any polypeptide for two or more nucleic acid sequences can be readily determined and compared to quantify the binding specificity of the polypeptide of interest with respect to a target nucleic acid sequence of interest. Binding of a nucleic acid-binding domain to a target nucleic acid sequence can be measured and detected in a variety of ways known in the art, including but not limited to assays using enzymatic or fluorescent labels, radiolabels, or gel shift assays.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences may also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) may be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm may be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14 (6): 6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP may be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs may be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

As various changes could be made in the above-described cells and methods without departing from the scope of the invention, it is intended that all matter contained in the above description and in the examples given below, shall be interpreted as illustrative and not in a limiting sense.

SEQUENCES

SEQ ID NO: 1
MeSWEET10a promoter
ATAAACGCTTCTCGCCCATC
SEQ ID NO: 2
DMS3-ZF

| | |
|---|---|
| LOCUS | DMS3 3804 bp DNA linear UNA 28-JUL-2021 |
| DEFINITION | . |
| ACCESSION | urn.local...d-dnzbu7j |
| VERSION | urn.local...d-dnzbu7j |
| KEYWORDS | . |
| SOURCE | |
| ORGANISM | . |
| FEATURES | Location/Qualifiers |
| misc_feature | 1..3804 |
| | /Transferred From = ″218. |
| | pEG302_BASTA_DMS3_Flag_ZF-SWEET1_2_mut_XhoI_HYG-DMS3″ |
| | /Transferred Similarity = ″100.00%″ |
| | /label = ″DMS3″ |
| origin | 243^{∧}244 |
| | /label = ″origin″ |

ORIGIN

| | |
|---|---|
| 1 | accgataaaa tgaaaatcat taacggagtc acgtggggtt cggttaaacg ccgttaagtg |
| 61 | tttttacgaa gttcacgagg tttctacttt tgtcttctct tattttaacg ccaatcgcct |
| 121 | ttatttccat ttgttataaa gtcttctgac atttgtaatg atcgaattct gtatttacat |
| 181 | atttgttata tcctatataa aatattattt gactttttct taaattttgt ggacttgtgg |
| 241 | acctattatg tttacatatt tttgatgaga agatgccata tttcgaatat atatgtacta |
| 301 | gtgtaatctg aggaaattca agttgaagcc aatacatact ttggaattta tatatttaaa |
| 361 | aacaaaaaaa aactatattc tataaataaa tttacttatt tactaattag tttgataaaa |
| 421 | ttcagttttt aataatactt ttatttgtat ggttaaatat gctacttaac aaaatatttt |
| 481 | taaatatata tattcatgag atttatcctt cctttatatc gagcttaaaa taaactattg |
| 541 | gtcacatgca tataatatgc atttatacat aaagttctga tttatatgat taacattggt |
| 601 | aacatgagtt tagaagcaat atttatccga cacattttaa agaatgaacg aaaaatttaa |
| 661 | aggtttcatt cgtttaggta tacatacaga atttactcaa gaactacaaa actttgatat |
| 721 | taaaatatca aatgatgccg atcatttgtc ttaagatgta aattaaaaga gagaaaaaaa |
| 781 | aacacaaaaa tgaaaatgtg cagttgtttt aataacggcg acctaacttt ctttatgagg |
| 841 | acccaagacc tacaccacct ccaaaattgg accacctttc gatttcacta ctcattgaga |
| 901 | gtctcataca tccacacgag tagctaaact attttttttt aagcataaac aaagctggaa |
| 961 | ttcatttttt ttttctttct aatgtaatca aagcttaaat tcaacattcc ttataaaata |
| 1021 | tgttataaat gataatgttt aggcaaaaac ataacatcat gtcacatgat ttttttttaa |
| 1081 | tagaaaacca gtacggogtt cgacaatatc gatacacaga gtcgttttcc taacgaacct |
| 1141 | aacgcatgac acagaactag ttgaatttgt aagataaaaa ctggtttgac atgtatcggt |
| 1201 | atcgaactca aatacgagaa attacctctg gttcgggttt tttcattatc aaactaatat |
| 1261 | gctacaaagt gagaaagtca ggctcagaag gaggtatgaa gcacaaagcc cacttggaat |
| 1321 | ctccgaagat tattgggcct taaataaggc cagaaaatat aattgaatga taaaaggaaa |
| 1381 | cttttcttac atttttggca aaatacgttc cttttattat tggtgtattt taatttagtt |
| 1441 | taattacttc acaacaattg gctgtttttc tccagaagaa cactaaagac aaaaattaaa |
| 1501 | ccctcacact cacagagacc ggagaagaag cagagagaaa gagagagaga gagattcacc |
| 1561 | ggtgataaca ttgaagcaga aagattctcc ggtgataaca ttgaagctga ggagcaatgt |
| 1621 | atccgactgg tcaacaggtt ggttgaaaaa aactcagttc aattgcaaaa tggaactaga |
| 1681 | ttagttggga aaaaacgaac gaactttagt ctatgatctc tottaaacga tgaagtctct |
| 1741 | ctgaattttt ctgtgaatct ggagaaaaga ctcgtgcttt attgtttatt gatgtatcta |
| 1801 | ctctcgacat tttgtttctt tttttattgc atgcgaccac attggattaa atttgagggt |
| 1861 | ttgtgtttca gttttttacg aattttaaga gttagggttt atacaggaat agtttttgag |
| 1921 | tcatttttgt atccaatttt gtagatttcg tttcagacca ctccgttgaa tgttcaagat |
| 1981 | ccaacaagga tgatgaattt ggatcaatct tcaccagttg caagaaatga aactcagaat |
| 2041 | ggaggaggca ttgctcacgc tgagtttgct atgttcaatt ccaagagget tgaatctgat |
| 2101 | cttgaagcta tgggtaacaa aatcaaacag catgaggata atctcaagtt tctcaagtct |
| 2161 | cagaaaaaca aaatggatga agctatcgtc gacttgcaag gttctgagat atcatagact |
| 2221 | tttacgtttc agtgatttat gttgattgag attatccaat atgtctttgt ttcgtttctt |
| 2281 | ctgcagttca tatgagtaag cttaactcgt ctcctactcc tagaagtgaa aactctgata |
| 2341 | acagtcttca gggtgaagat atcaatgcac agatccttcg ccacgaaaac tcagctgctg |
| 2401 | gagttttaag tctggttgag actcttcatg gtgctcaagc ttctcagttg atgttgacaa |
| 2461 | aaggtgttgt tggtgttgta gcgaaacttg ggaaggtcaa tgatgaaaat ctcagccagt |
| 2521 | tagttccatt tctctcactt tttcagatta ccttcaactt ctcaatgcca ctgattatat |
| 2581 | aagaaacctt tcttgcaatg gtttttgtga tctgaaacta actagttctc tctcgtggta |
| 2641 | ctttaaaaat gcaggatttt gtcgaattat ttggggactc gctcaatgtt ggcagttgta |
| 2701 | tgcaggaatt atgaaagtgt tacggcttta gaggcttatg ataaccatgg caacattgat |
| 2761 | ataaatgctg gccttcattg ccttggctct tcgattggaa gagagattgg agacagtttt |
| 2821 | gatgccatct gccttgaaaa cctgaggtac taatttttgt ttgaatcttt tacatgtatt |
| 2881 | acaaatttgg ttaggtacac ttcatctgca gtccttttct aattgcttca tttgatgact |
| 2941 | tttaggccgt atgttggtca gcacatagct gatgatctgc aaagaaggct tgatcttctg |
| 3001 | aaaccaaaat tacctaatgg tgagtgtcct cccgggtttc tcggatttgc agtgaatatg |
| 3061 | atacagatcg atccagctta cttgctctgt gtcacatcat atggatatgg tcttcgtgag |
| 3121 | accttgttct acaatctatt ctctcgcctt caagtttaca aaacaagggc cgatatgatt |
| 3181 | agtgccctcc catgcataag tgatggtgcg gtatctttgg atggaggaat catcaggaaa |
| 3241 | acggggatct tcaatcttgg aaaccggtaa gattctcaca attttataac ctctgaactt |
| 3301 | atttccctct ttggattctt ccatttcctt cttgctctta tccgctgtcc tgaactgaaa |
| 3361 | tcaatatgat tttcagtgat gaggtgaacg tgagatttgc aaagccaact gcttctcgca |
| 3421 | cgatggacaa ctatagtgag gcggagaaga aaatgaaaga gctgaaatgg aaaaaggaga |
| 3481 | aaacgctgga ggacattaag cgggagcaag tgctccgtga gcatgccgtc ttcaactttg |
| 3541 | gcaagaagaa agaagaattt gttcgatgct tggctcagag ttcatgcact aatcaggtat |
| 3601 | taaaactagt tttcatgtat cttctgaccc ttctcttcta tcatagtata tgtctgttga |
| 3661 | agcatatcat gaaattgtct atatgatcat atctttgttt cagttttaat tcaaagcttc |
| 3721 | aaatacctct atctgaaagt tttatatggc taacattggt ctgtttatgt gttggtggtg |
| 3781 | ccccagccaa tgaacacacc caga |

SEQ ID NO: 3

| | |
|---|---|
| LOCUS | gRNA4 20 bp DNA linear UNA 28-JUL-2021 |
| DEFINITION | dCAS9-MQ1 (Q147L). |
| ACCESSION | urn.local...s-dnzcc7n |
| VERSION | urn.local...s-dnzcc7n |
| KEYWORDS | . |
| SOURCE | |
| ORGANISM | . |
| FEATURES | Location/Qualifiers |
| gRNA | 1..20 |
| | /created by = ″User″ |
| | /label = ″gRNA suntag4″ |

ORIGIN

| | |
|---|---|
| 1 | ctagctatgt tgtgcaatga |

SEQ ID NO: 4
LOCUS gRNA5 20 bp DNA linear UNA 28-JUL-2021

| | |
|---|---|
| DEFINITION | dCAS9-MQ1 (Q147L). |
| ACCESSION | urn.local...z-dnzcd6a |
| VERSION | urn.local...z-dnzcd6a |
| KEYWORDS | . |
| SOURCE | |
| ORGANISM | . |
| FEATURES | Location/Qualifiers |
| gRNA | 1..20 |
| | / created_by = ″User″ |
| | /label = ″gRNA_suntag5″ |

ORIGIN

| | |
|---|---|
| 1 | taggggagga atccagggaa |

SEQ ID NO: 5

| | |
|---|---|
| LOCUS | MeSWEET20a Zinc Finger 504 bp DNA linear |
| | UNA 28-JUL-2021 |
| DEFINITION | . |
| ACCESSION | urn.local...k-dnzc0fv |
| VERSION | urn.local...k-dnzc0fv |
| KEYWORDS | . |
| SOURCE | |
| ORGANISM | . |
| FEATURES | Location/Qualifiers |
| misc_RNA | 1..504 |
| | /label=″SWEET1 2″ |

ORIGIN

| | |
|---|---|
| 1 | gagaaaccgt acaagtgtcc tgagtgcggt aaaagtttct ctaggagtga tgacctggtt |
| 61 | agacaccaaa ggacccacac aggcgagaag ccctataaat gtcctgaatg tggtaagagc |
| 121 | tttagtcaat ccagcaatct agtgcgtcat cagaggacgc ataccggcga aaagccctat |
| 181 | aaatgtcctg agagtggtaa aagtttctca caaagcggac atttaaccga gcatcagagg |
| 241 | actcatacag gggaaaaacc atacaaatgt cccgagtgcg gcaagtcatt tagtaggagt |
| 301 | gacaagctcg tgcgacacca acgaacccat acaggagaga agccatacaa atgtccggag |
| 361 | tgtggaaaaa gttttagtac atcaggaaat ttggtaagac atcagcgtac ccacaccggt |
| 421 | gagaaacctt acaaatgccc tgaatgtgga aagtctttca gtaggaggga tgagctgaat |
| 481 | gtccaccaga gaacgcatac gggg |

SEQ ID NO: 6

| | |
|---|---|
| LOCUS | MQ1_Q147L 1170 bp DNA linear UNA 28-JUL-2021 |
| DEFINITION | dCAS9-MQ1 (Q147L). |
| ACCESSION | urn.local...18-dnzci3k |
| VERSION | urn.local...18-dnzci3k |
| KEYWORDS | . |
| SOURCE | |
| ORGANISM | . |
| FEATURES | Location/Qualifiers |
| misc_structure | 1..1170 |
| | /label=″MQ1_Q147L″ |

ORIGIN

| | |
|---|---|
| 1 | gctagggaca gcaaagtgga gaacaaaaca aagaagctga gagtgttcga agccttcgcc |
| 61 | ggcattggcg cccagagaaa ggccctggag aaagtgagga aggacgagta cgagatcgtg |
| 121 | ggactggccg agtggtatgt gcccgccatc gtcatgtacc aggccatcca taacaacttc |
| 181 | cacaccaagc tcgagtacaa gtccgtcage agagaggaga tgatcgacta cctcgagaac |
| 241 | aagaccctgt cctggaacag caagaacccc gtcagcaacg gatactggaa gaggaagaag |
| 301 | gatgacgagc tgaagatcat ctacaacgcc atcaagctgt ccgaaaagga gggcaacatt |
| 361 | ttcgacatca gggacctcta caagaggaca ctgaagaaca tcgacctgct cacctacagc |
| 421 | ttcccttgcc aggacctgag ccagctaggc atccagaagg gcatgaagag gggaagcggc |
| 481 | accagatccg gcctgctctg ggagatcgaa agagccctgg actccaccga gaagaacgac |
| 541 | ctgcctaagt acctcctcat ggagaacgtg ggagccctgc tgcacaagaa gaacgaggag |
| 601 | gagctgaacc aatggaagca gaagctggag agcctgggct accagaacag catcgaagtc |
| 661 | ctcaatgctg ccgatttcgg atccagccag gccaggagga gagtgttcat gatctccacc |
| 721 | ctcaatgagt tcgtggaact gcctaagggc gacaagaagc ccaagagcat caaaaaggtg |
| 781 | ctgaacaaga tcgtgagcga gaaggacatc ctcaacaacc tgctgaaata caacctcacc |
| 841 | gaattcaaga agaccaagtc caacatcaac aaggccagcc tgatcggcta ctccaagttc |
| 901 | aactccgagg gctatgtgta cgaccccgag ttcacaggcc ccacactgac agctagcggc |
| 961 | gccaactcca ggatcaagat caaggacggc agcaacatca ggaagatgaa cagcgacgag |
| 1021 | accttcctgt acatcggctt tgacagccag gacggcaaga gagtgaacga aatcgagttc |
| 1081 | ctgaccgaga accagaagat cttcgtgtgt ggcaacagca tcagcgtgga ggtgctggag |
| 1141 | gccatcattg acaagatcgg cggccctagc |

SEQ ID NO: 7

| | |
|---|---|
| LOCUS | ntDRM2cd (catalytic domain) 1062 bp DNA linear UNA |
| | 28-JUL-2021 |
| DEFINITION | From Jacobsen lab (Ash), pEG302-1_22aaSunTag_insulator, |
| | longer linker,_UBQ10_scFv_DRMcd_NLS (June 2018). |
| ACCESSION | urn.local...1g-dnzcmnx |
| VERSION | urn.local...1g-dnzcmnx |
| KEYWORDS | . |
| SOURCE | |
| ORGANISM | . |
| FEATURES | Location/Qualifiers |
| misc_feature | 1..1062 |
| | /label = ″DRMcd″ |

ORIGIN

| | |
|---|---|
| 1 | gagacaattc gtttgcccaa acctatgatt gggtttgggg ttcctaccga accacttcca |
| 61 | gcaatggttc gaagaactct tcccgagcaa gctgttggac ccccgttttt ctactatgaa |
| 121 | aatgtggctc tagctccaaa gggtgtgtgg gacacaattt ctagattttt gtacgatatt |
| 181 | gagccagagt ttgtcgactc caaatatttt tgtgctgctg caagaaaaag gggttatatt |
| 241 | cataatctgc cggttgaaaa tagatttect ttgtttccac ttgccccacg taccattcat |
| 301 | gaggcacttc ccctatcgaa gaaatggtgg ccatcttggg acccacggac aaagttaaat |
| 361 | tgtttgcaaa cagccattgg aagtgcacaa ttgacgaata ggattaggaa agctgtggag |
| 421 | gactttgatg gtgagccacc aatgagagtt cagaagtttg ttcttgatca atgtcggaag |
| 481 | tggaatttgg tgtgggttgg gagaaacaaa gttgctcctt tggagcctga tgaagttgaa |
| 541 | atgctactgg gatttccaaa gaaccatact aggggaggtg gtataagtag gaccgataga |
| 601 | tacaagtcgc ttggtaattc attccaggtt gacactgtgg cataccattt gtcggtattg |
| 661 | aaagacttgt ttcccggtgg tatcaatgtc ttatcactct tctctggaat tggcggtggt |
| 721 | gaagttgctc tttatcgtct tggtattcca ctaaacacag tggtttctgt ggaaaaatct |
| 781 | gaagtcaaca gggatattgt gagaagctgg tgggagcaaa ctaatcagag agggaacctt |
| 841 | atacatttta acgatgtgca gcagctgaat ggagaccgct tggagcaact gatagagtca |
| 901 | tttggaggat ttgatttggt aattggtgga agcccgtgca ataaccttgc aggcagtaac |
| 961 | agagtgagca gggatgggct tgaaggcaaa gagtcttccc tattttatga ctatgttcgg |
| 1021 | atattggact tggtcaagtc cataatgtcc agacataaac at |

SEQ ID NO: 8
gRNA1

| | |
|---|---|
| LOCUS | Exported_-_9-248_gRNA 20 bp DNA linear UNA 28-JUL-2021 |
| DEFINITION | natural circular DNA. |
| ACCESSION | geneious\|urn:local:.:1x-dnzg1d8:0 |
| VERSION | geneious\|urn:local:.:1x-dnzg1d8:0 |
| KEYWORDS | . |
| SOURCE | null |
| ORGANISM | null |
| FEATURES | Location/Qualifiers |
| source | <1..>20 |
| | /organism = ″null″ |
| | /mol_type = ″genomic DNA″ |
| | /label = ″source null″ |
| misc_feature | 1..20 |
| | /label = ″9-248 gRNA″ |

ORIGIN

| | |
|---|---|
| 1 | cgctctcaac tgtacttcat |

SEQ ID NO: 9
gRNA2

| | |
|---|---|
| LOCUS | Exported_-_9-191_gRNA 20 bp DNA linear UNA 28-JUL-2021 |
| DEFINITION | natural circular DNA. |
| ACCESSION | geneious\|urn:local:.:1x-dnzg1d8:1 |
| VERSION | geneious\|urn:local:.:1x-dnzg1d8:1 |
| KEYWORDS | . |
| SOURCE | null |
| ORGANISM | null |
| FEATURES | Location/Qualifiers |
| source | <1..>20 |
| | /organism = ″null″ |
| | /mol_type = ″genomic DNA″ |
| | /label = ″source null″ |
| misc_feature | 1..20 |
| | /label = ″9-191 gRNA″ |

ORIGIN

| | |
|---|---|
| 1 | ccgatgaata agagcgctag |

SEQ ID NO: 10

| | |
|---|---|
| LOCUS | Exported_-_8-199_gRNA 20 bp DNA linear UNA 28-JUL-2021 |
| DEFINITION | natural circular DNA. |
| ACCESSION | geneious\|urn:local:.:1x-dnzg1d8:2 |
| VERSION | geneious\|urn:local:.1x-dnzg1d8:2 |
| KEYWORDS | . |
| SOURCE | null |
| ORGANISM | null |
| FEATURES | Location/Qualifiers |
| source | <1..>20 |
| | /organism = ″null″ |
| | /mol_type = ″genomic DNA″ |
| | /label = ″source null″ |
| misc_feature | 1..20 |
| | /label = ″8-199 gRNA″ |

ORIGIN

| | |
|---|---|
| 1 | agacgatgaa agagccgaag |

SEQ ID NO: 11

| | |
|---|---|
| LOCUS | Exported_-_8-128 gRNA 20 bp DNA linear UNA 28-JUL-2021 |
| DEFINITION | natural circular DNA. |
| ACCESSION | geneious\|urn:loca:.:1x-dnzg1d8:3 |
| VERSION | geneious\|urn:local:.:1x-dnzg1d8:3 |
| KEYWORDS | . |
| SOURCE | null |
| ORGANISM | null |
| FEATURES | Location/Qualifiers |
| source | <1. >20 |
| | /organism = ″null″ |
| | /mol_type = ″genomic DNA″ |
| | /label = ″source null″ |
| misc_feature | 1..20 |
| | /label = ″8-128 gRNA″ |

ORIGIN

| | |
|---|---|
| 1 | cactcgattg cagatttttg |

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. DNA Methylation of the MeSWEET10a Promoter Greatly Reduces the Binding Affinity of TAL20

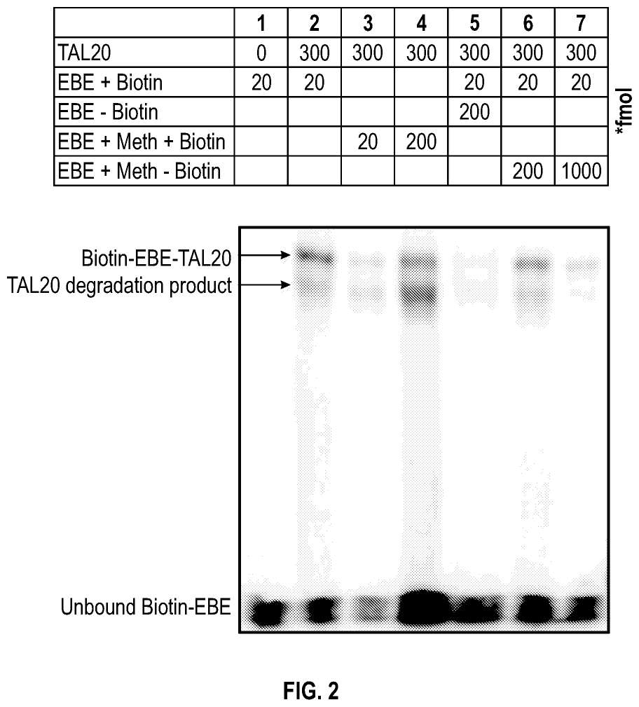

The methylation sensitivity of TALE protein binding to the MeSWEET10 promoter was experimentally demonstrated in vitro, using Electrophoretic Mobility Shift Assays (EMSAs) comparing methylated (CG methylation) to unmethylated DNA templates for TALE protein binding affinity (FIG. 2). It was found that the binding affinity of purified TAL20 to the MeSWEET10a promoter to be greatly reduced when the DNA was methylated at cytosines in the EBE sequence of the MeSWEET10a promoter. This confirms that DNA methylation is a viable strategy for preventing the induction of MeSWEET10a expression by TAL20. TAL20 proteins can be as described in Cohn et al., “Xanthomonas axonopodis Virulence Is Promoted by a Transcription Activator-Like Effector-Mediated Induction of a SWEET Sugar Transporter in Cassava”, MPMI Vol. 27, No. 11, 2014, pp. 1186-1198.

### Example 2. DMS3-ZF Expression Results in CpG Methylation at the MeSWEET10a Promoter EBE In Vivo

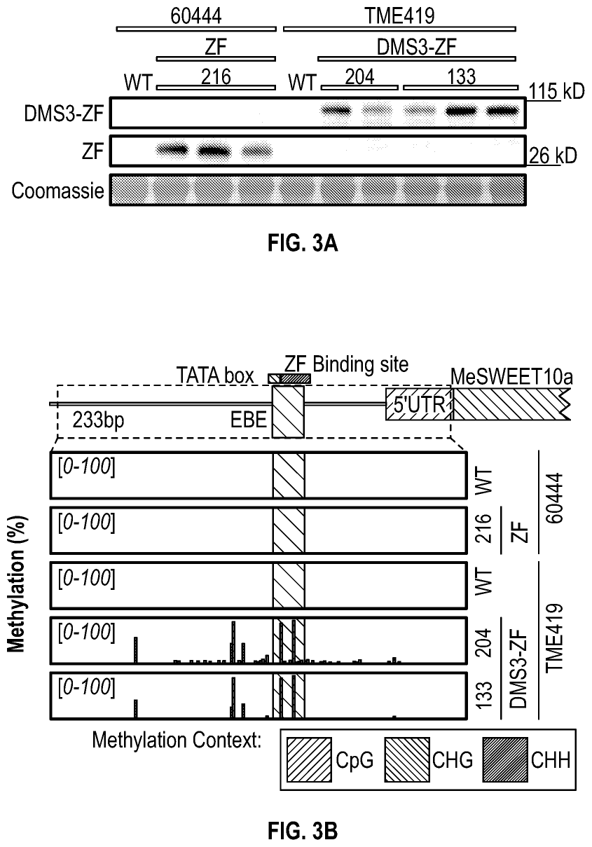

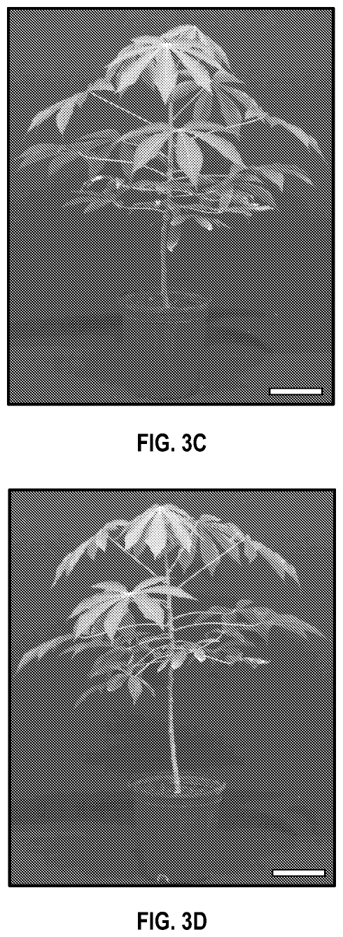

Two independent transgenic plant lines expressing DMS3-ZF (133 and 204) and a plant line expressing ZF-only negative control (216) were generated (FIG. 3A). The level of methylation of the promotor region of MeSweet10a was determined using PCR-based bisulfite sequencing (ampBS-seq). The results clearly show that DMS3-ZF specifically methylated the TAL 20 binding site (FIG. 3B). Plants expressing DMS3-ZF exhibited healthy growth and development (FIG. 3C and FIG. 3D).

### Example 3. DNA Methylation of the Binding Site of TAL20 in the MeSWEET10a Promoter Region Using dCas9-MQ1

An engineered DNA methylation system comprising the MQ1 (Q147L) (hereafter called MQ1v) bacterial CpG methyltransferase methylation protein from Mollicutes spiroplasma directly fused to dCas9 targeting protein (dCas9-MQ1v). The targeting protein is engineered to target MQ1 to the binding site of TAL20 in the MeSWEET10a S gene using a gRNA (gRNA4 and/or gRNA5) directed to target the engineered DNA methylation system to the binding site of TAL20 in the promoter region of MeSWEET10a. TAL20 is a TALE protein necessary for CBB infection. Deactivated MQ1 (dMQ1) and GFP fused to the dCas9 targeting protein were used as negative controls.

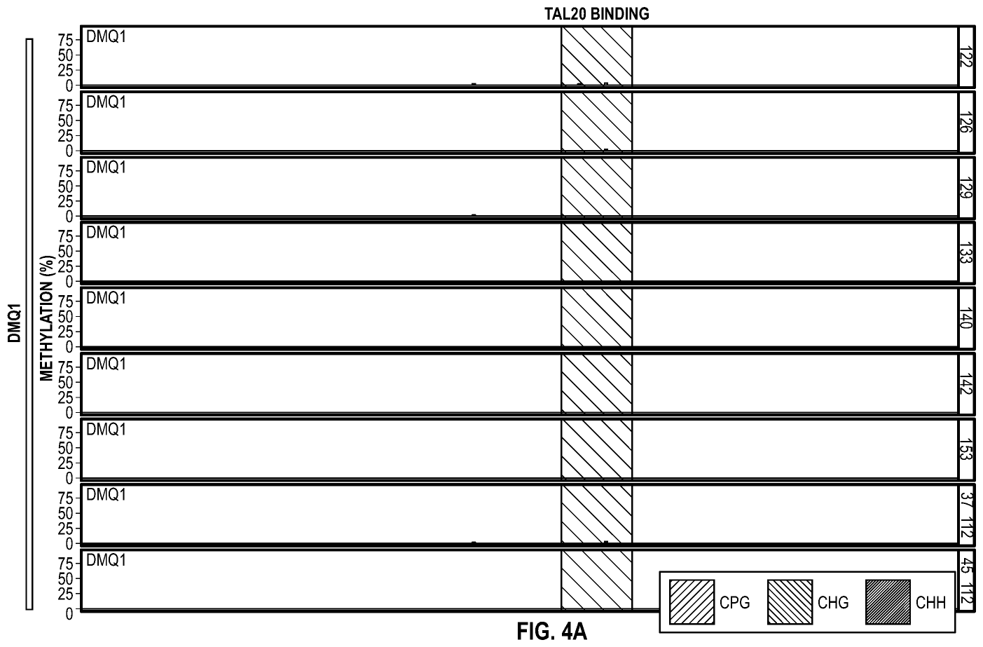

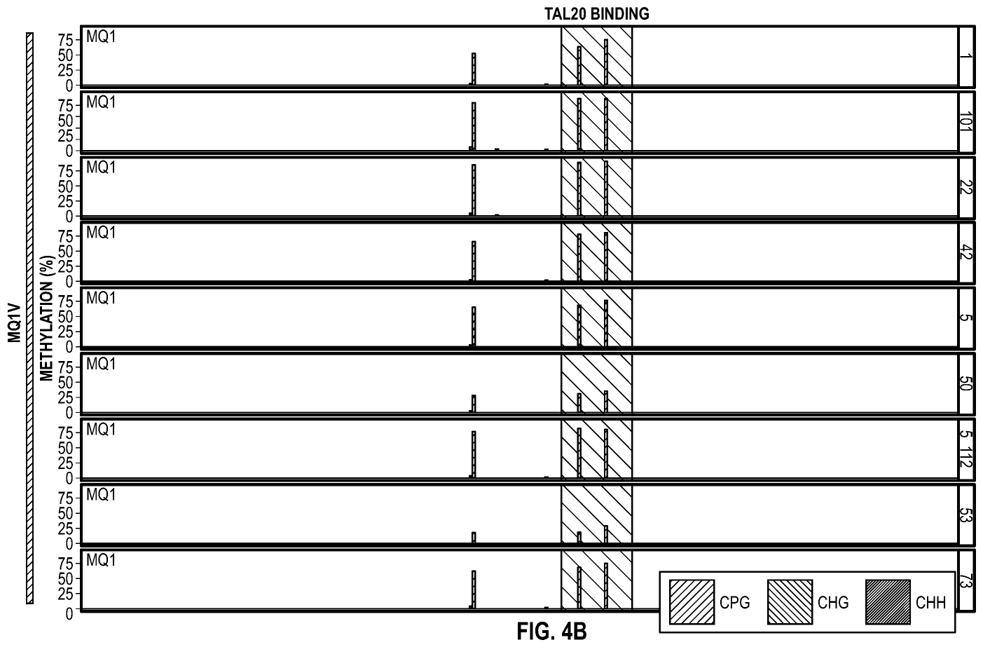

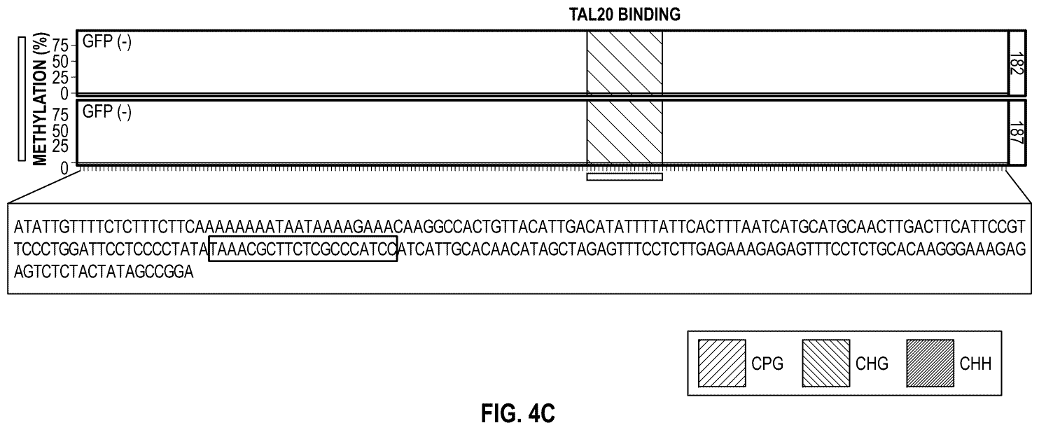

In short, nucleic acid constructs encoding the engineered DNA methylation system and controls were transformed into plant tissue culture cells, and the level of methylation at the TAL20 binding site was measured. As it is shown in FIG. 4, the dCas9-MQ1v system specifically methylated CpG sites at the TAL 20 binding site.

### Example 4. DNA Methylation of the Binding Site of Zinc Finger in the MeSWEET10a Promoter Region Using DMS3-ZF

An engineered DNA methylation system comprising the Arabidopsis thaliana DMS3 methylation protein directly fused to a zinc finger (ZF) targeting protein (DMS3-ZF). The ZF protein is engineered to target DMS3 to the binding site of TAL20 in the MeSWEET10a promoter region. The DMS3-ZF system specifically methylated CpG sites at the TAL 20 binding site in four transformed tissue lines (FIG. 4A-C). Cell line 133A showed the highest level of methylation.

### Example 5. Disease Phenotypes of Leaves from Plants Transformed with DMS3-ZF Directing Methylation to the Binding Site of TAL20

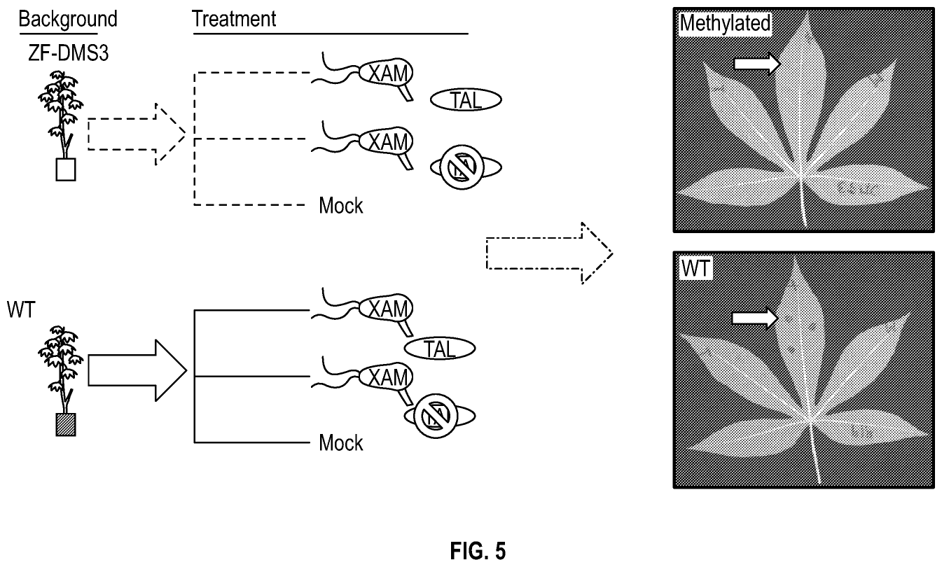

The effect of methylation on resistance of cassava plants to Cassava Xanthomonas axonopodis pv. manihotis, a causal agent of CBB, was tested. Plants were transformed with the DMS3-ZF methylation system described in Example 3, and susceptibility of the transformed plants to Xam was tested by infecting the plants with WT Xam. Untransformed (WT) plants and an Xam deletion mutant lacking TAL20 were used as controls. Mock-inoculated samples were also used as negative controls. Xam infection was assayed using water soaking, one of the earliest indicators of successful CBB infection by Xam. FIG. 5 shows that leaves with confirmed methylation lack water-soaking symptoms with WT Xam, indicating decreased susceptibility to Xam.

### Example 6. Cassava Plants with Methylation Targeted to the MeSWEET10a Promoter are More Resistant than Wildtype Controls

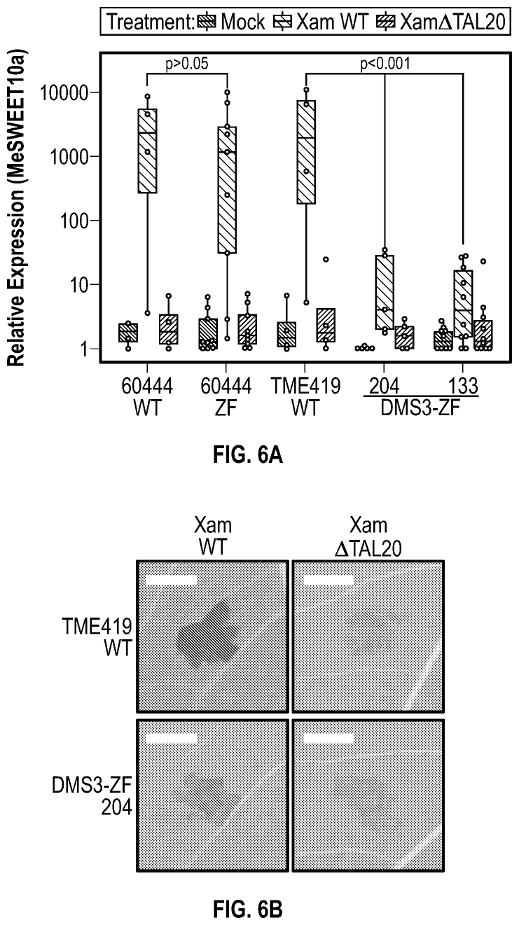

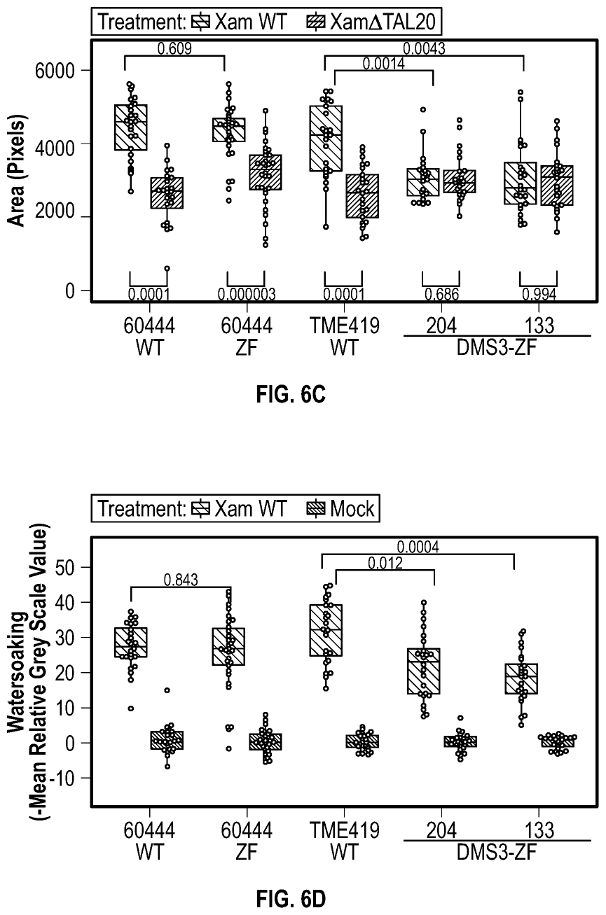

The level of resistance of the transgenic plants expressing DMS3-ZF was determined. Mock (no bacteria), ΔTAL20 (Xam668 mutant lacking TAL20) and wildtype Xam were infiltrated into wildtype (WT419) or MeSWEET10a promoter methylation mutant (DMS3) cassava leaves. FIG. 6A shows that induction of expression of MeSWEET10a in plants expressing DMS3-ZF in response to Xam infiltration was significantly reduced when compared to WT and ZF-only plants.

Six days after inoculation, lesion size was quantified using ImageJ. ΔTAL20 mutant caused similar sized lesions on WT419 and DMS3 cassava. Wildtype Xam caused significantly smaller lesions on DMS3 cassava as compared to WT419 cassava as observed in images of FIG. 6B, and as quantified using pixel measurements of observed are of water-soaking (FIG. 6C), the intensity of water-soaking phenotype (FIG. 6D).

### Example 7. DNA Methylation of the Binding Site of TAL20 in the MeSWEET10a Promoter Region Using SunTag-DRM2

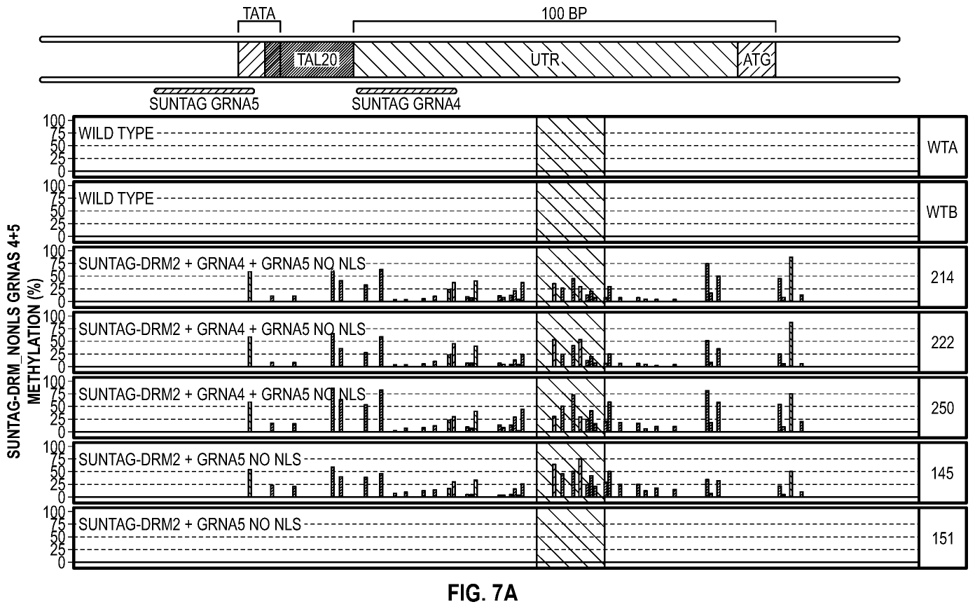

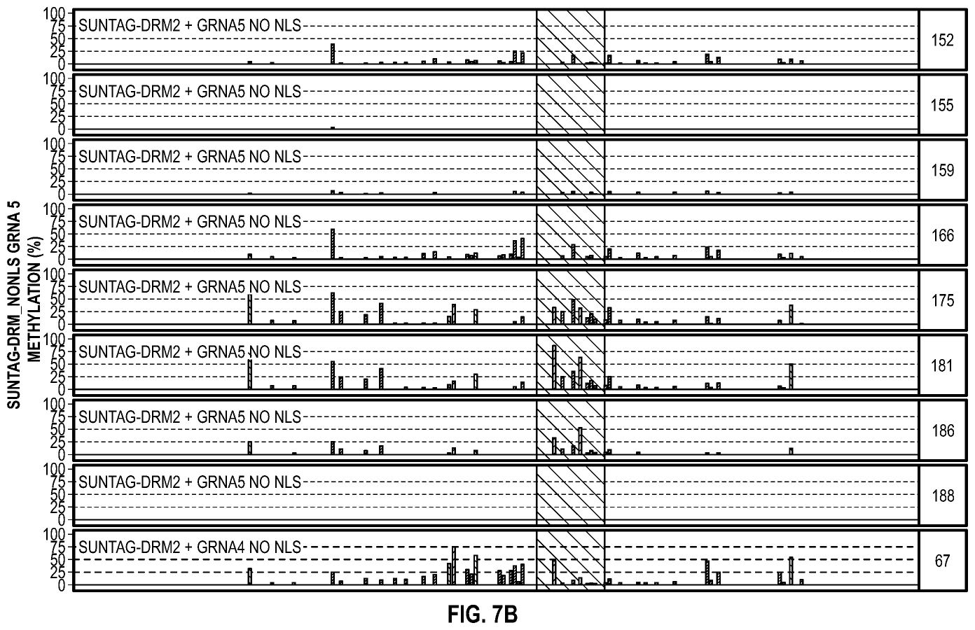

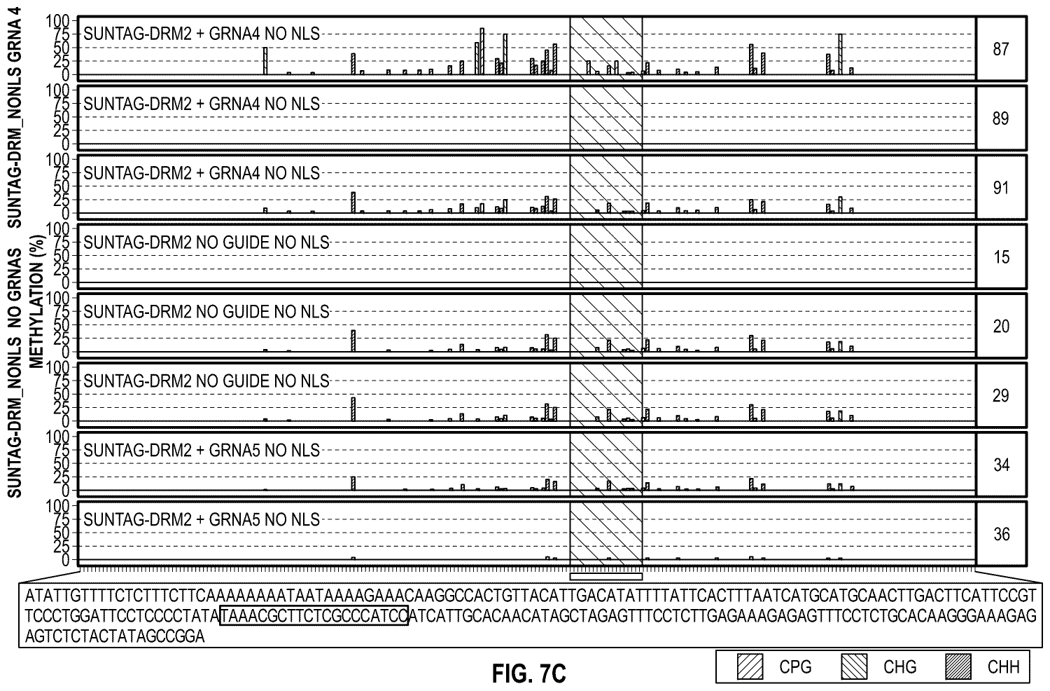

An engineered DNA methylation system comprising the Nicotiana tabacum DRM2 (cd) methylation protein using a dCas9-based SunTag DNA methylation system (SunTag-DRM2) to direct methylation to the binding site of TAL20 in the MeSWEET10a promoter region. Two gRNAs (gRNA4 and gRNA5) were used to each direct a SunTag-DRM2 (SunTag-DRM2_noNLS gRNA 4; SunTag-DRM2_noNLS gRNA 5) to a different methylation locus in the promoter region of MeSWEET10a. The two systems (gRNA4 and gRNA5 systems) were used individually or together to direct methylation. As it is shown in FIG. 7A-C, the SunTag-DRM2 system methylated the TAL20 binding site in transformed tissue lines when compared to controls. Further, an increased level of methylation was observed when the two systems (gRNA4 and gRNA5 systems) are used together when compared to the level of methylation when each system is used individually.

### Example 8. Effect of CRISPR-Targeted Methylation on CBB Disease Phenotypes in Cassava

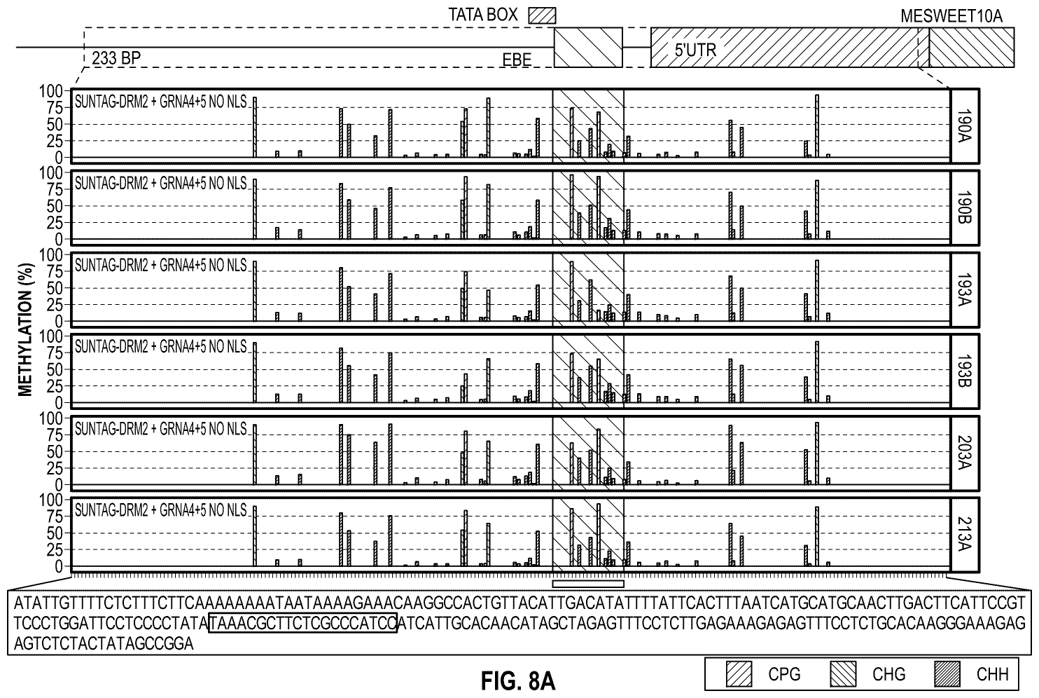

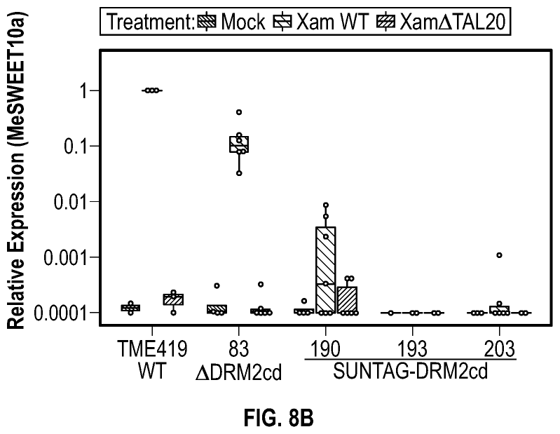

Methylation at the binding site of TAL20 (grey) using SunTag-DRM was measured. As shown in FIG. 8A, cassava plants with methylation targeted to the MeSWEET10a promoter show reduced transcriptional activation in CBB disease challenge experiments. Upon challenge with Xam, plants expressing SunTag-DRM showed significantly reduced expression of MeSWEET10a (FIG. 8B).

### Example 9. DNA Methylation of the Promoter Region of nCBP1 Using SunTag-DRM2

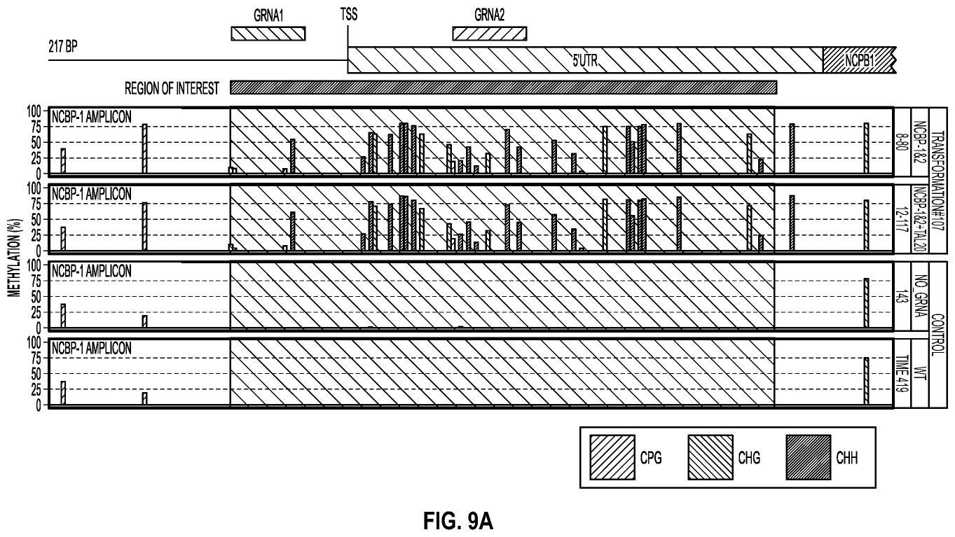

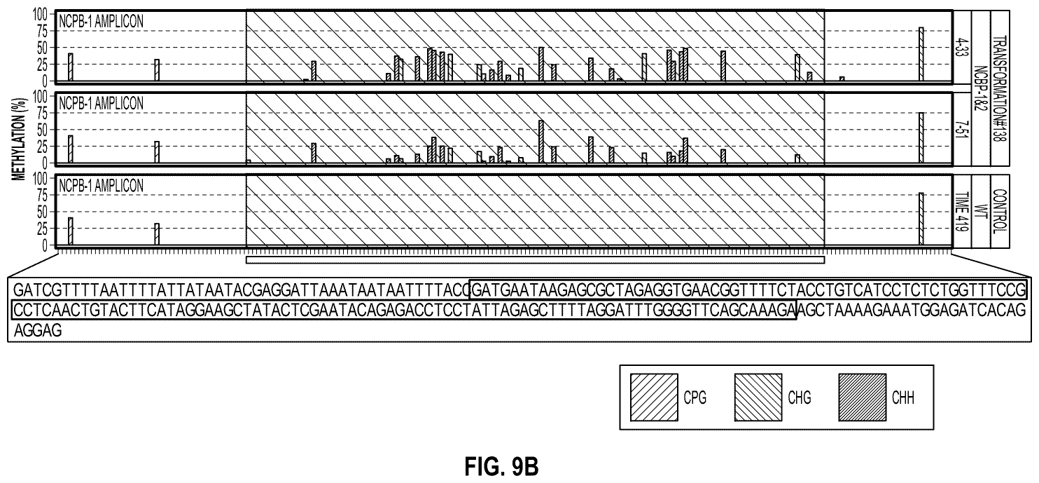

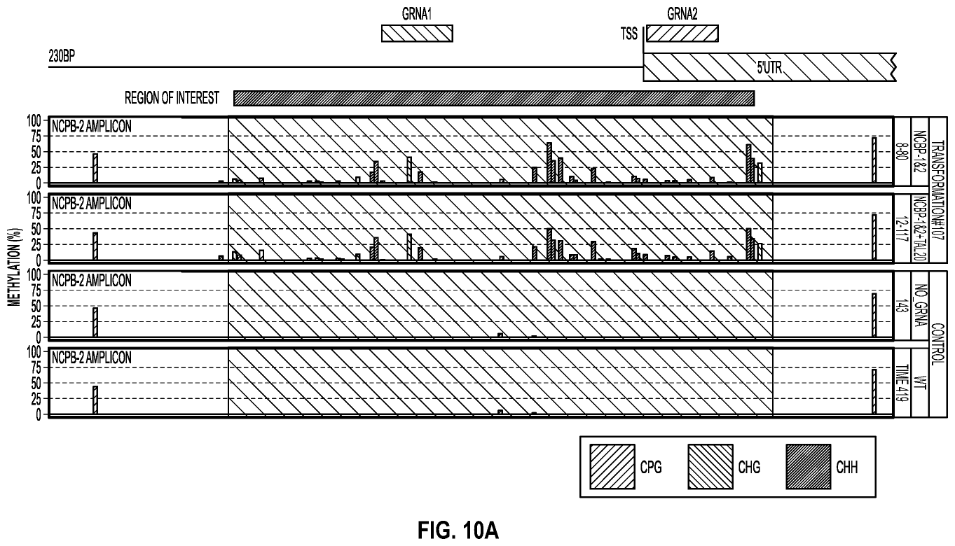

An engineered DNA methylation system comprising the Arabidopsis thaliana DRM methylation protein using a dCas9-based SunTag engineered DNA methylation system (SunTag-DRM) to direct methylation to the promoter region of the nCBP1 gene. Two gRNAs (gRNA1 and gRNA2) were used to each direct a SunTag-DRM2 (SunTag-DRM2_noNLS gRNA 1; SunTag-DRM2_noNLS gRNA 2) to a different methylation locus in the promoter region of nCBP1. As it is shown in FIG. 9A-B, each SunTag-DRM system methylated the TAL20 binding site in transformed tissue lines when compared to controls.

### Example 10. DNA Methylation of the Promoter Region of nCBP2 Using SunTag-DRM2

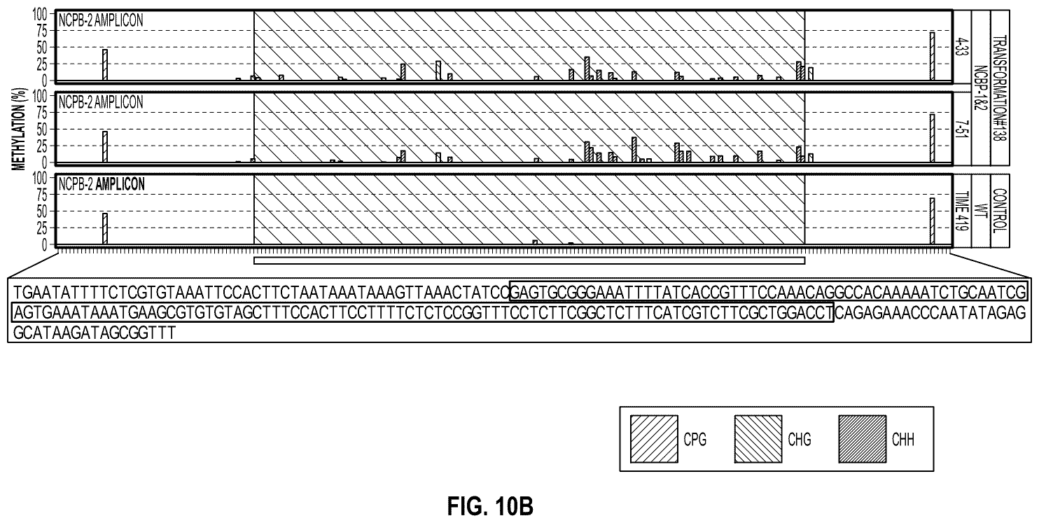

An engineered DNA methylation system comprising the Arabidopsis thaliana DRM methylation protein using a dCas9-based SunTag engineered DNA methylation system (SunTag-DRM2) to direct methylation to the promoter region of the nCBP2 gene. Two gRNAs (gRNA1 and gRNA2) were used to each direct a SunTag-DRM2 (SunTag-DRM2 gRNA 1; SunTag-DRM2 gRNA 2) to a different methylation locus in the promoter region of nCBP2. As it is shown in FIG. 10A-B, each SunTag-DRM2 system methylated the TAL20 binding site in transformed tissue lines when compared to controls.

### Example 11. Tissue-Specific Methylation Targeting of MeSWEET10a in Cassava

An engineered DNA methylation system is engineered to methylate the promoter of MeSWEET10a in cassava. The engineered DNA methylation system is specifically expressed in leaves under the control of a leaf-specific promoter. Epigenetically modified cassava plants are generated having reduced expression of MeSWEET10a. The plants exhibited healthy growth and development and are resistant to CBB.

### Example 12. Testing for the Inheritance of Silencing of the MeSWEET10a Gene, and the Inheritance of CBB Resistance

To test the heritability of methylation at the TAL20-binding site, crossing blocks are established. Pairwise crosses are performed between three epigenetically modified cassava lines from different backgrounds to generate three F1 populations. The populations are examined for methylation at target loci, clonally propagated, and further assessed for CBB susceptibility and TAL-effector dependent expression of susceptibility genes at DDPSC. As with the parent plants, the progeny cassava plants comprising methylated loci are resistant to CBB.

### Example 13. Testing for the Inheritance of Silencing of the eIF4E Genes, and the Inheritance of CBSV Resistance

CBSV resistant transgenic cassava plants comprising methylated promoters of eIF4E genes are generated. The resistant plants are crossed to segregate away the methylation-targeting transgene to test for inheritance of the DNA methylation and CBSV resistance. As with the parent plants, the progeny cassava plants comprising methylated loci are resistant to CBSV.

### Example 14. Combining H3K4Me3 Removal with Methylation Targeting

The inventors discovered that the histone mark H3 lysine 4 tri-methylation (H3K4me3) acts antagonistically to DNA methylation. The inventors also discovered that one of the components of RNA-directed DNA methylation, SHH1, is specifically repelled by this mark. H3K4me3 is removed in cassava plants, and the promoter of an S gene is methylated in these plants. Methylation is more effective in plants where H3K4me3 is removed when compared to plants where H3K4me3 is present.

### Example 15. Direct Targeting of CG Methylation

The bacterial CG-specific Sssl methyltransferase was successfully used in Arabidopsis to methylate promoters of disease-resistant plants. However, this methyltransferase had broad genome wide off-target effects. However, a mutant form of Sssl called MQ1 Q147L was recently reported that shows reduced overall activity, resulting in reduced off-target methylation. This mutant shows targeted DNA methylation at a plant gene with no off-target effects.

What is claimed is:

1. An expression construct for methylating a target nucleic acid sequence in a plant pathogen susceptibility gene, the expression construct comprising a promoter operably linked to a nucleic acid sequence encoding an engineered protein, wherein the engineered protein comprises a methylation polypeptide comprising a DNA methylation domain of a DNA methylation protein linked to a targeting polypeptide comprising a sequence-specific DNA binding domain, wherein the DNA binding domain binds a target DNA sequence in a target methylation locus in a polynucleotide encoding a plant pathogen susceptibility gene.

2. The expression construct of claim 1, wherein binding of the DNA binding domain to the target DNA sequence targets the engineered protein to the target locus, thereby mediating methylation of one or more methylation sites in the target locus, thereby modulating the expression of the plant pathogen susceptibility gene.

3. The expression construct of claim 1 or 2, wherein the targeting polypeptide is fused to the methylation polypeptide.

4. The expression construct of any one of the preceding claims, wherein the targeting polypeptide comprises an epitope and the methylation polypeptide comprises an affinity polypeptide that specifically binds to the epitope, and wherein binding of the affinity polypeptide to the epitope links the targeting polypeptide to the methylation polypeptide.

5. The expression construct of claim 4, wherein the epitope is multimerized.

6. The expression construct of any of the preceding claims, wherein the targeting polypeptide is a programmable targeting protein comprising a programmable, sequence-specific DNA-binding domain.

7. The expression construct of claim 6, wherein the programmable targeting polypeptide is an RNA-guided clustered regularly interspersed short palindromic repeats (CRISPR)/CRISPR-associated (Cas) (CRISPR/Cas) nuclease system, a zinc finger nuclease (ZFN), a transcription activator-like effector nuclease (TALEN), a meganuclease, a ssDNA-guided Argonaute endonuclease, a meganuclease, a rare-cutting endonuclease, or any combination thereof.

8. The expression construct of claim 6, wherein the programmable targeting protein is a CRISPR/Cas nuclease system comprising a nuclease-deficient CAS9 protein (dCAS9) and a guide RNA (gRNA).

9. The expression construct of one of claims 1-7, wherein the programmable targeting protein is a zinc finger DNA binding domain.

10. The expression construct of one of claims 1-7, wherein the targeting polynucleotide comprises a TALE protein.

11. The expression construct of any one of the preceding claims, wherein the engineered protein comprises more than one methylation polypeptide linked to a targeting polypeptide programmed to target the more than one methylation polypeptide to the target methylation loci.

12. The expression construct of any one of the preceding claims, wherein the engineered protein comprises a methylation polypeptide and more than one targeting polypeptide engineered to bind one or more target DNA sequence.

13. The expression construct of any one of the preceding claims, wherein the engineered protein mediates methylation of more than one target methylation locus.

14. The expression construct of any one of the preceding claims, wherein the engineered protein modulates the expression of more than one plant pathogen susceptibility gene.

15. The expression construct of any one of the preceding claims, wherein the methylation polypeptide methylates CpG, CpHpG, or CpHpH methylation sites, or any combination thereof.

16. The expression construct of any one of the preceding claims, wherein the methylation polypeptide methylates CpG, CpHpG, or CpHpH methylation sites, or any combination thereof to thereby remove histone proteins.

17. The expression construct of any one of the preceding claims, wherein the engineered protein comprises a DNA methylation domain of a methylation protein selected from SUVH2, SUVH9, DMS3, DRM2, DRM3, NRPE1, NRPD1, CLSY1, NRPD2, RDR2, DCL3, AGO4, DRD1, RDM1, DMS4, KTF1, IDN2, SUVR2, MQ1, and any combination thereof.

18. The expression construct of any one of the preceding claims, wherein the engineered protein comprises a DNA methylation domain of a DMS3 protein.

19. The expression construct of claim 18, wherein the DMS3 protein is encoded by a nucleic acid sequence comprising at least about 75% or more, at least about 85% or more, at least about 95% or more, or 100% sequence identity with a nucleic acid sequence of SEQ ID NO: 2.

20. The expression construct of any one of claims 1-17, wherein the engineered protein comprises a DNA methylation domain of a DRM2 protein.

21. The expression construct of claim 20, wherein the DRM2 protein is encoded by a nucleic acid sequence comprising at least about 75% or more, at least about 85% or more, at least about 95% or more, or 100% sequence identity with a nucleic acid sequence of SEQ ID NO: 7.

22. The expression construct of one of claims 1-17, wherein the engineered protein comprises a DNA methylation domain of a MQ1 protein.

23. The expression construct of claim 22, wherein the MQ1 protein is encoded by a nucleic acid sequence comprising at least about 75% or more, at least about 85% or more, at least about 95% or more, or 100% sequence identity with a nucleic acid sequence of SEQ ID NO: 6.

24. The expression construct of any one of the preceding claims, wherein the pathogen is a viral, bacterial, oomycete, animal, fungal pathogen, or any combination thereof.

25. The expression construct of any one of the preceding claims, wherein the pathogen is a viral pathogen.

26. The expression construct of any one of the preceding claims, wherein the pathogen is a bacterial pathogen.

27. The expression construct of any one of the preceding claims, wherein the plant is cassava.

28. The expression construct of claim 27, wherein the plant pathogen susceptibility gene is MeSWEET10a.

29. The expression construct of one of claims 1-28, wherein the plant is cassava, the susceptibility gene is MeSWEET10a, and the pathogen is a bacterial pathogen that causes cassava bacterial blight (CBB).

30. The expression construct of claim 29, wherein the pathogen that causes CBB is a Xanthomonas sp.

31. The expression construct of one of claims 1-28, wherein the plant is cassava, the plant pathogen susceptibility gene is MeSWEET10a, and the pathogen is a Xanthomonas sp.

32. The expression construct of claim 31, wherein the plant pathogen susceptibility gene is nCBP-1, nCBP-2, or combinations thereof.

33. The expression construct of claim 31, wherein the plant pathogen susceptibility gene is nCBP-1 and nCBP-2.

34. The expression construct of one of claims 1-28, wherein the plant is cassava, the plant pathogen susceptibility gene is nCBP-1 and nCBP-2, and the pathogen is a viral pathogen that causes cassava brown streak disease.

35. The expression construct of claim 34, wherein the viral pathogen that causes cassava brown streak disease is selected from cassava brown streak virus (CBSV), Uganda CBSV, or a combination thereof.

36. The expression construct of one of claims 1-28, wherein the plant is cassava, the susceptibility gene is nCBP-1 and nCBP-2, and the pathogen is CBSV.

37. The expression construct of claim 1, wherein the engineered protein comprises a methylation polypeptide comprising a DNA methylation domain of a DMS3 protein fused to a zinc finger DNA binding domain programmed to target the engineered protein to a locus in a promoter region of a cassava MeSWEET10a gene.

38. The expression construct of claim 37, wherein the DMS3 protein (or methylation polypeptide) is encoded by a nucleic acid sequence comprising at least about 75% or more, at least about 85% or more, at least about 95% or more, or 100% sequence identity with a nucleic acid sequence of SEQ ID NO: 2 and wherein the programmable targeting protein (or targeting polypeptide) comprises an amino acid sequence comprising at least about 75% or more, at least about 85% or more, at least about 95% or more, or 100% sequence identity with the amino acid sequence of SEQ ID NO: 5.

39. The expression construct of claim 1, wherein the engineered protein comprises a methylation polypeptide comprising a DNA methylation domain of a MQ1 protein fused to a nuclease-deficient CAS9 protein (dCAS9) of a CRISPR/Cas nuclease system comprising a gRNA comprising a sequence which binds to a nucleotide sequence in the target nucleic acid sequence to thereby target the engineered protein to a locus in a promoter region of a cassava MeSWEET10a gene.

40. The expression construct of claim 39, wherein the MQ1 protein is encoded by a nucleic acid sequence comprising at least about 75% or more, at least about 85% or more, at least about 95% or more, or 100% sequence identity with a nucleic acid sequence of SEQ ID NO: 6 and wherein the gRNA is selected from a gRNA selected from a gRNA comprising SEQ ID NO: 3, a gRNA comprising SEQ ID NO: 4, or a combination thereof.

41. The expression construct of claim 1, wherein the engineered protein comprises a DRM2 methylation polypeptide comprising an affinity polypeptide and a dCAS9 protein of a CRISPR/Cas nuclease system comprising a gRNA comprising a sequence which binds to a nucleotide sequence in the target nucleic acid sequence to thereby target the engineered protein to a locus in a promoter region of a cassava MeSWEET10a gene, wherein the dCas9 protein comprises an epitope that specifically binds to the affinity polypeptide.

42. The expression construct of claim 41, wherein the gRNA is selected from a gRNA selected from a gRNA comprising the nucleic acid sequence of SEQ ID NO: 3, a gRNA comprising the nucleic acid sequence of SEQ ID NO: 4, or a combination thereof.

43. The expression construct of claim 1, wherein the engineered protein comprises a DRM2 methylation polypeptide comprising an affinity polypeptide and a dCAS9 protein of a CRISPR/Cas nuclease system comprising a gRNA comprising a sequence which binds to a nucleotide sequence in the target nucleic acid sequence to thereby target the engineered protein to a locus in a promoter region of a cassava nCBP1 gene, wherein the dCas9 protein comprises a multimerized epitope that specifically binds to the affinity polypeptide.

44. The expression construct of claim 43, wherein the gRNA is selected from a gRNA selected from a gRNA comprising the nucleic acid sequence of SEQ ID NO: 8, a gRNA comprising the nucleic acid sequence of SEQ ID NO: 9, or a combination thereof.

45. The expression construct of claim 1, wherein the engineered protein comprises a DRM2 methylation polypeptide comprising an affinity polypeptide and a dCAS9 protein of a CRISPR/Cas nuclease system comprising a gRNA comprising a sequence which binds to a nucleotide sequence in the target nucleic acid sequence to thereby target the engineered protein to a locus in a promoter region of a cassava nCBP2 gene, wherein the dCas9 protein comprises a multimerized epitope that specifically binds to the affinity polypeptide.

46. The expression construct of claim 45, wherein the gRNA is selected from a gRNA selected from a gRNA comprising the nucleic acid sequence of SEQ ID NO: 10, a gRNA comprising the nucleic acid sequence of SEQ ID NO: 11, or a combination thereof.

47. An expression construct for methylating a target nucleic acid sequence in a plant pathogen susceptibility gene, the expression construct comprising a promoter operably linked to a nucleic acid sequence encoding an engineered protein, wherein the engineered protein comprises:
a. a programmable targeting polypeptide comprising a programmable sequence-specific DNA binding domain, wherein the programmable DNA binding domain binds a target DNA sequence in a target methylation locus in a polynucleotide encoding a plant pathogen susceptibility gene, wherein the programmable targeting protein comprises:
i. a nuclease-deficient CAS9 protein (dCAS9) and optionally an epitope; and
ii. one or more guide RNA; and
b. a methylation polypeptide comprising a DNA methylation domain of a DRM2 protein, a DMS3 protein, or an MQ1 protein;
wherein the methylation polypeptide is fused to the targeting polypeptide or fused to an affinity polypeptide that specifically binds to the epitope.

48. An expression construct for methylating a target nucleic acid sequence in a plant pathogen susceptibility gene, the expression construct comprising a promoter operably linked to a nucleic acid sequence encoding an engineered protein, wherein the engineered protein comprises:
a. a programmable targeting polypeptide comprising a programmable sequence-specific DNA binding domain of a zinc finger DNA binding protein programmed to specifically bind one or more target DNA sequence in a target methylation locus in a polynucleotide encoding a plant pathogen susceptibility gene, wherein the targeting polypeptide optionally comprises an epitope; and
b. a methylation polypeptide comprising a DNA methylation domain of a DMS3 protein, a DRM2 protein, an MQ1 protein, wherein the methylation polypeptide is fused to the targeting polypeptide or fused to an affinity polypeptide that specifically binds to the epitope.

49. An expression construct for methylating a target nucleic acid sequence in a plant pathogen susceptibility gene, the expression construct comprising a promoter operably linked to a nucleic acid sequence encoding an engineered protein, wherein the engineered protein comprises:
a. a programmable targeting polypeptide comprising a programmable sequence-specific DNA binding domain of a TALE DNA binding protein programmed to specifically bind one or more target DNA sequence in a target methylation locus in a polynucleotide encoding a plant pathogen susceptibility gene, wherein the targeting polypeptide optionally comprises an epitope; and
b. a methylation polypeptide comprising a DNA methylation domain of a DMS3 protein, a DRM2 protein, an MQ1 protein, wherein the methylation polypeptide is fused to the targeting polypeptide or fused to an affinity polypeptide that specifically binds to the epitope.

50. One or more vectors comprising one or more expression constructs of any of claims 1-49 for methylating a target nucleic acid sequence in a plant pathogen susceptibility gene.

51. A plant or plant cell comprising one or more expression constructs of any of claims 1-49, or one or more one or more vectors of claim 50.

52. A plant or plant cell comprising one or more methylated sites in a methylation locus in a plant pathogen susceptibility gene.

53. The plant or plant cell of claim 52, wherein the plant is cassava.

54. The plant or plant cell of claim 53, wherein the plant pathogen susceptibility gene is MeSWEET10a.

55. The plant or plant cell of claim 52, wherein the plant is cassava, the susceptibility gene is MeSWEET10a, and the pathogen is a bacterial pathogen that causes cassava bacterial blight (CBB).

56. The plant or plant cell of claim 55, wherein the pathogen that causes CBB is a Xanthomonas sp.

57. The plant or plant cell of claim 52, wherein the plant is cassava, the plant pathogen susceptibility gene is MeSWEET10a, and the pathogen is a Xanthomonas sp.

58. The plant or plant cell of claim 53, wherein the plant pathogen susceptibility gene is nCBP-1, nCBP-2, or combinations thereof.

59. The plant or plant cell of claim 53, wherein the plant pathogen susceptibility gene is nCBP-1 and nCBP-2.

60. The expression construct of one of claim 52, wherein the plant is cassava, the plant pathogen susceptibility gene is nCBP-1 and nCBP-2, and the pathogen is a viral pathogen that causes cassava brown streak disease.

61. The expression construct of claim 60, wherein the viral pathogen that causes cassava brown streak disease is selected from cassava brown streak virus (CBSV), Uganda CBSV, or a combination thereof.

62. The expression construct of one of claim 52, wherein the plant is cassava, the susceptibility gene is nCBP-1 and nCBP-2, and the pathogen is CBSV.

63. A disease-resistant cassava plant, the cassava plant comprising one or more methylated sites in a promoter region of a MeSWEET10a susceptibility gene, wherein the cassava plant is resistant to a Xanthomonas sp. that causes cassava bacterial blight (CBB).

64. A disease-resistant cassava plant, the cassava plant comprising one or more methylated sites in a promoter region of an nCBP-1 gene susceptibility and one or more methylated sites in a promoter region an nCBP-2 susceptibility gene, wherein the cassava plant is resistant to a viral pathogen that causes cassava brown streak disease.

65. The disease-resistant cassava plant of claim 64, wherein the viral pathogen that causes cassava brown streak disease is selected from cassava brown streak virus (CBSV), Uganda CBSV, or a combination thereof.

66. A disease-resistant cassava plant, the cassava plant comprising one or more methylated sites in a promoter region of an nCBP-1 gene susceptibility and an nCBP-2 susceptibility gene, wherein the cassava plant is resistant to CBSV.

67. A method of generating a disease resistant or tolerant plant, the method comprising:
a. introducing one or more expression constructs of any of claims 1-49, or one or more one or more vectors of claim 50 into a plant or plant cell;
b. cultivating the plant or plant cell under conditions sufficient for the engineered protein is targeted to the target methylation loci in the one or more plant pathogen susceptibility genes, thereby generating an engineered plant or plant cell comprising one or more methylated loci, thereby generating the disease resistant or tolerant plant; and
C. optionally removing the one or more expression or one or more one or more vectors from the plant or plant cell.

68. The method of claim 67, wherein the plant is cassava.

69. The method of claim 68, wherein the susceptibility gene is MeSWEET10a.

70. The method of claim 67, wherein the plant is cassava, the susceptibility gene is MeSWEET10a, and the pathogen is a bacterial pathogen that causes cassava bacterial blight (CBB).

71. The method of claim 70, wherein the pathogen that causes CBB is a Xanthomonas sp.

72. The method of claim 67, wherein the plant is cassava, the susceptibility gene is MeSWEET10a, and the pathogen is a Xanthomonas sp.

73. The method of claim 72, wherein the susceptibility gene is nCBP-1, nCBP-2, or both.

74. The method of claim 73, wherein the susceptibility gene is nCBP-1 and nCBP-2.

75. The method of claim 67, wherein the plant is cassava, the susceptibility gene is nCBP-1 and nCBP-2, and the pathogen is a viral pathogen that causes cassava brown streak disease.

76. The method of claim 75, wherein the viral pathogen that causes cassava brown streak disease is selected from cassava brown streak virus (CBSV), Uganda CBSV, and a combination thereof.

77. The method of claim 67, wherein the plant is cassava, the susceptibility gene is nCBP-1 and nCBP-2, and the pathogen is CBSV.

78. A kit for generating an epigenetically modified plant, plant part, or plant cell, the kit comprising one or more expression constructs of any of claims 1-49, one or more vectors of claim 50, or any combination thereof, or one or more plants, plant parts, plant cell culture, or plant cells comprising the one or more expression constructs, one or more vectors, or any combination thereof.
